SEQUENCE LISTING (104 sequences)

SEQ ID NO: 1 - protein, 105 aa, Dengue virus 4
GMSYTMCSGKFSIDKEMAETQHGTTVVKVKYEGAGAPCKVPIEIRDVNKEKVVGRIISSTPFAENTNSVTNIELEPPFGDSYIVIGVGDSALTLHWFRKGSSIGK

SEQ ID NO: 2 - DNA, 315 bp, Dengue virus 4
ggaatgtcatacacgatgtgttcaggaaagttttcaattgacaaagagatggcagaaacacagcatgggacaacagtggtgaaagtcaagtatgaaggtgctggagctccgtgtaaagtccccatagagataagagatgtaaacaaggaaaaagtggttgggcgtatcatctcatccaccccttttgctgagaataccaacagtgtaaccaacatagaattagaacccccctttggggacagctacatagtgataggtgttggagatagcgcattaacactccattggttcaggaaagggagttccattggcaag

SEQ ID NO: 3 - protein, 105 aa, Artificial Sequence
  source: /note=“Description of Artificial Sequence Synthetic polypeptide”
GMSAAMCSGKFSIDKEMAETEAGTTVVKVKYEGAGAPCKVPIEIRDVNKEKVVGRIISSTPFAENTNSVTNIELEPPFGDSYIVIGVGDSALTLHWFREGSSIGK

SEQ ID NO: 4 - protein, 105 aa, Artificial Sequence
  source: /note=“Description of Artificial Sequence Synthetic polypeptide”
GMSAAMCSGKFSIDKEMAETEHGTTVVKVKYEGAGAPCKVPIEIRDVNKEKVVGRIISSTPFAENTNSVTNIELEPPFGDSYIVIGVGDSALTLHWFRKGSSIGK

SEQ ID NO: 5 - protein, 105 aa, Artificial Sequence
  source: /note=“Description of Artificial Sequence Synthetic polypeptide”
GMSATMCSGKFSIDKEMAETPDGTTVVKVKYEGAGAPCKVPIEIRDADTEKVVGAIISSTPFAENTNSVTNIELEPPFGDSYIVIGVGDSALTLHWFREGSSIGK

SEQ ID NO: 6 - protein, 105 aa, Artificial Sequence
  source: /note=“Description of Artificial Sequence Synthetic polypeptide”
GMSDAMCSGKFSIDKEMAATEHGTTVVKVKYEGAGAPCKVPIEIRDATTEAVVGGIISSTPFAENTNSVTNIELEPPFGDSYIVIGVGDSALTLHWFREGSSIGK

SEQ ID NO: 7 - protein, 105 aa, Artificial Sequence
  source: /note=“Description of Artificial Sequence Synthetic polypeptide”
GMSDAMCSGKFSIDKEMAATPHGTTVVKVKYEGAGAPCKVPIEIRDAAKEKVVGGIISSTPFAENTNSVTNIELEPPFGDSYIVIGVGDSALTLHWFREGSSIGK

SEQ ID NO: 8 - protein, 105 aa, Artificial Sequence
  source: /note=“Description of Artificial Sequence Synthetic polypeptide”
GMSDAMCSGKFSIDKEMAETEDGTTVVKVKYEGAGAPCKVPIEIRDVNKEKVVGRIISSTPFAENTNSVTNIELEPPFGDSYIVIGVGDSALTLHWFREGSSIGK

SEQ ID NO: 9 - protein, 105 aa, Artificial Sequence
  source: /note=“Description of Artificial Sequence Synthetic polypeptide”
GMSDAMCSGKFSIDKEMAETPDGTTVVKVKYEGAGAPCKVPIEIRDVNKEKVVGRIISSTPFAENTNSVTNIELEPPFGDSYIVIGVGDSALTLHWFREGSSIGK

SEQ ID NO: 10 - protein, 105 aa, Artificial Sequence
  source: /note=“Description of Artificial Sequence Synthetic polypeptide”
GMSSAMCSGKFSIDKEMAATEAGTTVVKVKYEGAGAPCKVPIEIRDADKAKVVGGIISSTPFAENTNSVTNIELEPPFGDSYIVIGVGDSALTLHWFREGSSIGK

SEQ ID NO: 11 - protein, 105 aa, Artificial Sequence
  source: /note=“Description of Artificial Sequence Synthetic polypeptide”
GMSYAMCSGKFSIDKEMAATHDGTTVVKVKYEGAGAPCKVPIEIRDVNKEKVVGRIISSTPFAENTNSVTNIELEPPFGDSYIVIGVGDSALTLHWFRAGSSIGK

SEQ ID NO: 12 - protein, 105 aa, Artificial Sequence
  source: /note=“Description of Artificial Sequence Synthetic polypeptide”
GMSYTMCSGKFSIDKEMAATEHGTTVVKVKYEGAGAPCKVPIEIRDATKEKVVGGIISSTPLAENTNSVTNIELEPPFGDSYIVIGVGNSALTLHWFREGSSIGK

SEQ ID NO: 13 - protein, 105 aa, Artificial Sequence
  source: /note=“Description of Artificial Sequence Synthetic polypeptide”
GMSYTMCSGKFSIDKEMAEAEDGTTVVKVKYEGAGAPCKVPIEIRDADEETVVGRIISSTPLAENTNSVTNIELEPPFGDSYIVIGVGNSALTLHWFREGSSIGK

SEQ ID NO: 14 - protein, 105 aa, Dengue virus 4; identical to SEQ ID NO: 1

SEQ ID NO: 15 - DNA, 315 bp, Dengue virus 4
ggaatgtcatacacgatgtgttcaggaaagttttcaattgacaaagagatggcagaaacacagcatgggacaacagtggtgaaagtcaagtatgaaggtgctggagctccgtgtaaagtccccatagagataagagatgtaaacaaggaaaaagtggttgggcgtatcatctcatccacccctttggctgagaataccaacagtgtaaccaacatagaattagaacccccctttggggacagctacatagtgataggtgttggaaacagcgcattaacactccattggttcaggaaagggagttccattggcaag

SEQ ID NO: 16 - protein, 108 aa, Zika virus
GVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGK

SEQ ID NO: 17 - DNA, 324 bp, Zika virus
ggtgtgagctatagcctgtgcaccgccgccttcaccttcaccaaaattccggccgaaaccctgcatggcaccgtgaccgtggaagtgcagtatgccggtaccgatggtccgtgcaaagttccggcccagatggccgtggatatgcagaccctgaccccggtgggtcgcctgattaccgccaatccggtgattaccgagagcaccgagaacagcaagatgatgctggaactggatccgccgttcggtgatagctatatcgtgattggcgtgggcgagaagaagatcacccatcattggcatcgcagcggtagcaccatcggcaaa

SEQ ID NO: 18 - protein, 108 aa, Artificial Sequence
  source: /note=“Description of Artificial Sequence Synthetic polypeptide”
GVSYSLCTAAFTFAKTPAETLHGTVTVEVQYAGTDGPCKVPAQMAVDMAAATPVGRLITANPVITESTENSKMVLELDPPFGDSYIVIGVGEKKITHHWHRSGSTIGK

SEQ ID NO: 19 - protein, 108 aa, Artificial Sequence
  source: /note=“Description of Artificial Sequence Synthetic polypeptide”
GVSYSLCTAAFTFTAIPAETLHGTVTVEVQYAGTDGPCKVPAQMAVDMQAATPVGRLITANPVITESTENSKMVLELDPPFGDSYIVIGVGEKKITHHWHRSGSTIGK

SEQ ID NO: 20 - protein, 108 aa, Artificial Sequence
  source: /note=“Description of Artificial Sequence Synthetic polypeptide”
GVSYSLCTAAFTFAKAPAETLHGTVAVEVQYAGTDGPCKVPAQMAVDMAAATPVGRLITANPVITESTENSKMTLELDPPFGDSYIVIGVGEKKITHHWHRSGSTIGK

SEQ ID NO: 21 - protein, 108 aa, Artificial Sequence
  source: /note=“Description of Artificial Sequence Synthetic polypeptide”
GVSYSLCTAAFTFATAPAETLHGTVTVEVQYAGTDGPCKVPAQMAVAMQATTPVGRLITANPVITESTENSKMTLELDPPFGDSYIVIGVGEKKITHHWHRSGSTIGK

SEQ ID NO: 22 - protein, 108 aa, Artificial Sequence
  source: /note=“Description of Artificial Sequence Synthetic polypeptide”
GVSYSLCTAAFTFAKAPAETLHGTVAVEVQYAGTDGPCKVPAQMAVDMQAATPVGRLITANPVITESTENSAMVLELDPPFGDSYIVIGVGEKKITHHWHRSGSTIGK

SEQ ID NO: 23 - protein, 108 aa, Artificial Sequence
  source: /note=“Description of Artificial Sequence Synthetic polypeptide”
GVSYSLCTAAFTFTVEPAETLHGTVTVEVQYAGTDGPCKVPAQMAVDMETATPVGRLITANPVITESTENSKMMLELDPPFGDSYIVIGVGEKKITHHWHRSGSTIGK

SEQ ID NO: 24 - protein, 108 aa, Artificial Sequence
  source: /note=“Description of Artificial Sequence Synthetic polypeptide”
GVSYSLCTAAFTFTEAPAETLHGTVTVAVQYAGTDGPCKVPAQMAVDMETATPVGRLITANPVITESTENSKMVLELDPPFGDSYIVIGVGEKKITHHWHRSGSTIGK

SEQ ID NO: 25 - protein, 108 aa, Artificial Sequence
  source: /note=“Description of Artificial Sequence Synthetic polypeptide”
GVSYSLCTAAFTFATIPAETLHGTVAVEVQYAGTDGPCKVPAQMAVEMETATPVGRLITANPVITESTENSKMMLELDPPFGDSYIVIGVGEKKITHHWHRSGSTIGK

SEQ ID NO: 26 - protein, 108 aa, Artificial Sequence
  source: /note=“Description of Artificial Sequence Synthetic polypeptide”
GVSYSLCTAAFTFAAIPAETLHGTVAVAVQYAGTDGPCKVPAQMAVDMPTATPVGRLITANPVITESTENSKMTLELDPPFGDSYIVIGVGEKKITHHWHRSGSTIGK

SEQ ID NO: 27 - protein, 108 aa, Artificial Sequence
  source: /note=“Description of Artificial Sequence Synthetic polypeptide”
GVSYSLCTAAFTFAAAPAETLHGTVAVEVQYAGTDGPCKVPAQMAVDMEAATPVGRLITANPVITESTENSKMALELDPPFGDSYIVIGVGEKKITHHWHRSGSTIGK

SEQ ID NO: 28 - protein, 108 aa, Artificial Sequence; identical to SEQ ID NO: 16
  source: /note=“Description of Artificial Sequence Synthetic polypeptide”

SEQ ID NO: 29 - DNA, 315 bp, Dengue virus 4
ggaatgtcatacacgatgtgttcaggaaagttttcaattgacaaagagatggcagaaacacagcatgggacaacagtggtgaaagtcaagtatgaaggtgctggagctccgtgtaaagtccccatagagataagagatgtaaacaaggaaaaagtggttgggcgtatcatctcatccaccccttttgctgagaataccaacagtgtaaccaacatagaattagaacccccctttggggacagctacatagtgataggtgttggagacagcgcattaacactccattggttcaggaaagggagttccattggcaag

SEQ ID NO: 30 - DNA, 315 bp, Dengue virus 4
ggaatgtcatacacgatgtgttcaggaaagttttcaattgacaaagagatggcagaaacacagcatgggacaacagtggtgaaagtcaagtatgaaggtgctggagctccgtgtaaagtccccatagagataagagatgtaaacaaggaaaaagtggttgggcgtatcatctcatccacccctttcgctgagaataccaacagtgtaaccaacatagaattagaacccccctttggggacagctacatagtgataggtgttggagatagcgcattaacactccattggttcaggaaagggagttccattggcaag

SEQ ID NO: 31 - DNA, 315 bp, Dengue virus 4
ggaatgtcatacacgatgtgttcaggaaagttttcaattgacaaagagatggcagaaacacagcatgggacaacagtggtgaaagtcaagtatgaaggtgctggagctccgtgtaaagtccccatagagataagagatgtaaacaaggaaaaagtggttgggcgtatcatctcatccacccctttcgctgagaataccaacagtgtaaccaacatagaattagaacccccctttggggacagctacatagtgataggtgttggagacagcgcattaacactccattggttcaggaaagggagttccattggcaag

SEQ ID NO: 32 - DNA, 315 bp, Artificial Sequence
  source: /note=“Description of Artificial Sequence Synthetic polynucleotide”
ggcatgagcgccgccatgtgcagcggcaagttcagcatcgacaaggagatggccgagaccgaggccggcaccaccgtggtgaaggtgaagtacgagggcgccggcgccccctgcaaggtgcccatcgagatcagggacgtgaacaaggagaaggtggtgggcaggatcatcagcagcacccccttcgccgagaacaccaacagcgtgaccaacatcgagctggagccccccttcggcgacagctacatcgtgatcggcgtgggcgacagcgccctgaccctgcactggttcagggagggcagcagcatcggcaag

SEQ ID NO: 33 - DNA, 315 bp, Artificial Sequence
  source: /note=“Description of Artificial Sequence Synthetic polynucleotide”
ggcatgagcgccgccatgtgcagcggcaagttcagcatcgacaaggagatggccgagaccgagcacggcaccaccgtggtgaaggtgaagtacgagggcgccggcgccccctgcaaggtgcccatcgagatcagggacgtgaacaaggagaaggtggtgggcaggatcatcagcagcacccccttcgccgagaacaccaacagcgtgaccaacatcgagctggagccccccttcggcgacagctacatcgtgatcggcgtgggcgacagcgccctgaccctgcactggttcaggaagggcagcagcatcggcaag

SEQ ID NO: 34 - DNA, 315 bp, Artificial Sequence
  source: /note=“Description of Artificial Sequence Synthetic polynucleotide”
ggcatgagcgccaccatgtgcagcggcaagttcagcatcgacaaggagatggccgagacccccgacggcaccaccgtggtgaaggtgaagtacgagggcgccggcgccccctgcaaggtgcccatcgagatcagggacgccgacaccgagaaggtggtgggcgccatcatcagcagcacccccttcgccgagaacaccaacagcgtgaccaacatcgagctggagccccccttcggcgacagctacatcgtgatcggcgtgggcgacagcgccctgaccctgcactggttcagggagggcagcagcatcggcaag

SEQ ID NO: 35 - DNA, 315 bp, Artificial Sequence
  source: /note=“Description of Artificial Sequence Synthetic polynucleotide”
ggcatgagcgacgccatgtgcagcggcaagttcagcatcgacaaggagatggccgccaccgagcacggcaccaccgtggtgaaggtgaagtacgagggcgccggcgccccctgcaaggtgcccatcgagatcagggacgccaccaccgaggccgtggtgggcggcatcatcagcagcacccccttcgccgagaacaccaacagcgtgaccaacatcgagctggagccccccttcggcgacagctacatcgtgatcggcgtgggcgacagcgccctgaccctgcactggttcagggagggcagcagcatcggcaag

SEQ ID NO: 36 - DNA, 315 bp, Artificial Sequence
  source: /note=“Description of Artificial Sequence Synthetic polynucleotide”
ggcatgagcgacgccatgtgcagcggcaagttcagcatcgacaaggagatggccgccaccccccacggcaccaccgtggtgaaggtgaagtacgagggcgccggcgccccctgcaaggtgcccatcgagatcagggacgccgccaaggagaaggtggtgggcggcatcatcagcagcacccccttcgccgagaacaccaacagcgtgaccaacatcgagctggagccccccttcggcgacagctacatcgtgatcggcgtgggcgacagcgccctgaccctgcactggttcagggagggcagcagcatcggcaag

SEQ ID NO: 37 - DNA, 315 bp, Artificial Sequence
  source: /note=“Description of Artificial Sequence Synthetic polynucleotide”
ggcatgagcgacgccatgtgcagcggcaagttcagcatcgacaaggagatggccgagaccgaggacggcaccaccgtggtgaaggtgaagtacgagggcgccggcgccccctgcaaggtgcccatcgagatcagggacgtgaacaaggagaaggtggtgggcaggatcatcagcagcacccccttcgccgagaacaccaacagcgtgaccaacatcgagctggagccccccttcggcgacagctacatcgtgatcggcgtgggcgacagcgccctgaccctgcactggttcagggagggcagcagcatcggcaag

SEQ ID NO: 38 - DNA, 315 bp, Artificial Sequence
  source: /note=“Description of Artificial Sequence Synthetic polynucleotide”
ggcatgagcgacgccatgtgcagcggcaagttcagcatcgacaaggagatggccgagacccccgacggcaccaccgtggtgaaggtgaagtacgagggcgccggcgccccctgcaaggtgcccatcgagatcagggacgtgaacaaggagaaggtggtgggcaggatcatcagcagcacccccttcgccgagaacaccaacagcgtgaccaacatcgagctggagccccccttcggcgacagctacatcgtgatcggcgtgggcgacagcgccctgaccctgcactggttcagggagggcagcagcatcggcaag

SEQ ID NO: 39 - DNA, 315 bp, Artificial Sequence
  source: /note=“Description of Artificial Sequence Synthetic polynucleotide”
ggcatgagcagcgccatgtgcagcggcaagttcagcatcgacaaggagatggccgccaccgaggccggcaccaccgtggtgaaggtgaagtacgagggcgccggcgccccctgcaaggtgcccatcgagatcagggacgccgacaaggccaaggtggtgggcggcatcatcagcagcacccccttcgccgagaacaccaacagcgtgaccaacatcgagctggagccccccttcggcgacagctacatcgtgatcggcgtgggcgacagcgccctgaccctgcactggttcagggagggcagcagcatcggcaag

SEQ ID NO: 40 - DNA, 315 bp, Artificial Sequence
  source: /note=“Description of Artificial Sequence Synthetic polynucleotide”
ggcatgagctacgccatgtgcagcggcaagttcagcatcgacaaggagatggccgccacccacgacggcaccaccgtggtgaaggtgaagtacgagggcgccggcgccccctgcaaggtgcccatcgagatcagggacgtgaacaaggagaaggtggtgggcaggatcatcagcagcacccccttcgccgagaacaccaacagcgtgaccaacatcgagctggagccccccttcggcgacagctacatcgtgatcggcgtgggcgacagcgccctgaccctgcactggttcagggccggcagcagcatcggcaag

SEQ ID NO: 41 - DNA, 315 bp, Artificial Sequence
  source: /note=“Description of Artificial Sequence Synthetic polynucleotide”
ggcatgagctacaccatgtgcagcggcaagttcagcatcgacaaggagatggccgccaccgagcacggcaccaccgtggtgaaggtgaagtacgagggcgccggcgccccctgcaaggtgcccatcgagatcagggacgccaccaaggagaaggtggtgggcggcatcatcagcagcacccccctggccgagaacaccaacagcgtgaccaacatcgagctggagccccccttcggcgacagctacatcgtgatcggcgtgggcaacagcgccctgaccctgcactggttcagggagggcagcagcatcggcaag

SEQ ID NO: 42 - DNA, 315 bp, Artificial Sequence
  source: /note=“Description of Artificial Sequence Synthetic polynucleotide”
ggcatgagctacaccatgtgcagcggcaagttcagcatcgacaaggagatggccgaggccgaggacggcaccaccgtggtgaaggtgaagtacgagggcgccggcgccccctgcaaggtgcccatcgagatcagggacgccgacgaggagaccgtggtgggcaggatcatcagcagcacccccctggccgagaacaccaacagcgtgaccaacatcgagctggagccccccttcggcgacagctacatcgtgatcggcgtgggcaacagcgccctgaccctgcactggttcagggagggcagcagcatcggcaag

SEQ ID NO: 43 - DNA, 315 bp, Dengue virus 4
ggcatgagctacaccatgtgcagcggcaagttcagcatcgacaaggagatggccgagacccagcacggcaccaccgtggtgaaggtgaagtacgagggcgccggcgccccctgcaaggtgcccatcgagatcagggacgtgaacaaggagaaggtggtgggcaggatcatcagcagcacccccttcgccgagaacaccaacagcgtgaccaacatcgagctggagccccccttcggcgacagctacatcgtgatcggcgtgggcgacagcgccctgaccctgcactggttcaggaagggcagcagcatcggcaag

SEQ ID NO: 44 - DNA, 324 bp, Artificial Sequence
  source: /note=“Description of Artificial Sequence Synthetic polynucleotide”
ggcgtgagctacagcctgtgcaccgccgccttcaccttcgccaagacccccgccgagaccctgcacggcaccgtgaccgtggaggtgcagtacgccggcaccgacggcccctgcaaggtgcccgcccagatggccgtggacatggccgccgccacccccgtgggcaggctgatcaccgccaaccccgtgatcaccgagagcaccgagaacagcaagatggtgctggagctggacccccccttcggcgacagctacatcgtgatcggcgtgggcgagaagaagatcacccaccactggcacaggagcggcagcaccatcggcaag

SEQ ID NO: 45 - DNA, 324 bp, Artificial Sequence
  source: /note=“Description of Artificial Sequence Synthetic polynucleotide”
ggcgtgagctacagcctgtgcaccgccgccttcaccttcaccgccatccccgccgagaccctgcacggcaccgtgaccgtggaggtgcagtacgccggcaccgacggcccctgcaaggtgcccgcccagatggccgtggacatgcaggccgccacccccgtgggcaggctgatcaccgccaaccccgtgatcaccgagagcaccgagaacagcaagatggtgctggagctggacccccccttcggcgacagctacatcgtgatcggcgtgggcgagaagaagatcacccaccactggcacaggagcggcagcaccatcggcaag

SEQ ID NO: 46 - DNA, 324 bp, Artificial Sequence
  source: /note=“Description of Artificial Sequence Synthetic polynucleotide”
ggcgtgagctacagcctgtgcaccgccgccttcaccttcgccaaggcccccgccgagaccctgcacggcaccgtggccgtggaggtgcagtacgccggcaccgacggcccctgcaaggtgcccgcccagatggccgtggacatggccgccgccacccccgtgggcaggctgatcaccgccaaccccgtgatcaccgagagcaccgagaacagcaagatgaccctggagctggacccccccttcggcgacagctacatcgtgatcggcgtgggcgagaagaagatcacccaccactggcacaggagcggcagcaccatcggcaag

SEQ ID NO: 47 - DNA, 324 bp, Artificial Sequence
  source: /note=“Description of Artificial Sequence Synthetic polynucleotide”
ggcgtgagctacagcctgtgcaccgccgccttcaccttcgccaccgcccccgccgagaccctgcacggcaccgtgaccgtggaggtgcagtacgccggcaccgacggcccctgcaaggtgcccgcccagatggccgtggccatgcaggccaccacccccgtgggcaggctgatcaccgccaaccccgtgatcaccgagagcaccgagaacagcaagatgaccctggagctggacccccccttcggcgacagctacatcgtgatcggcgtgggcgagaagaagatcacccaccactggcacaggagcggcagcaccatcggcaag

SEQ ID NO: 48 - DNA, 324 bp, Artificial Sequence
  source: /note=“Description of Artificial Sequence Synthetic polynucleotide”
ggcgtgagctacagcctgtgcaccgccgccttcaccttcgccaaggcccccgccgagaccctgcacggcaccgtggccgtggaggtgcagtacgccggcaccgacggcccctgcaaggtgcccgcccagatggccgtggacatgcaggccgccacccccgtgggcaggctgatcaccgccaaccccgtgatcaccgagagcaccgagaacagcgccatggtgctggagctggacccccccttcggcgacagctacatcgtgatcggcgtgggcgagaagaagatcacccaccactggcacaggagcggcagcaccatcggcaag

SEQ ID NO: 49 - DNA, 324 bp, Artificial Sequence
  source: /note=“Description of Artificial Sequence Synthetic polynucleotide”
ggcgtgagctacagcctgtgcaccgccgccttcaccttcaccgtggagcccgccgagaccctgcacggcaccgtgaccgtggaggtgcagtacgccggcaccgacggcccctgcaaggtgcccgcccagatggccgtggacatggagaccgccacccccgtgggcaggctgatcaccgccaaccccgtgatcaccgagagcaccgagaacagcaagatgatgctggagctggacccccccttcggcgacagctacatcgtgatcggcgtgggcgagaagaagatcacccaccactggcacaggagcggcagcaccatcggcaag

SEQ ID NO: 50 - DNA, 324 bp, Artificial Sequence
  source: /note=“Description of Artificial Sequence Synthetic polynucleotide”
ggcgtgagctacagcctgtgcaccgccgccttcaccttcaccgaggcccccgccgagaccctgcacggcaccgtgaccgtggccgtgcagtacgccggcaccgacggcccctgcaaggtgcccgcccagatggccgtggacatggagaccgccacccccgtgggcaggctgatcaccgccaaccccgtgatcaccgagagcaccgagaacagcaagatggtgctggagctggacccccccttcggcgacagctacatcgtgatcggcgtgggcgagaagaagatcacccaccactggcacaggagcggcagcaccatcggcaag

SEQ ID NO: 51 - DNA, 324 bp, Artificial Sequence
  source: /note=“Description of Artificial Sequence Synthetic polynucleotide”
ggcgtgagctacagcctgtgcaccgccgccttcaccttcgccaccatccccgccgagaccctgcacggcaccgtggccgtggaggtgcagtacgccggcaccgacggcccctgcaaggtgcccgcccagatggccgtggagatggagaccgccacccccgtgggcaggctgatcaccgccaaccccgtgatcaccgagagcaccgagaacagcaagatgatgctggagctggacccccccttcggcgacagctacatcgtgatcggcgtgggcgagaagaagatcacccaccactggcacaggagcggcagcaccatcggcaag

SEQ ID NO: 52 - DNA, 324 bp, Artificial Sequence
  source: /note=“Description of Artificial Sequence Synthetic polynucleotide”
ggcgtgagctacagcctgtgcaccgccgccttcaccttcgccgccatccccgccgagaccctgcacggcaccgtggccgtggccgtgcagtacgccggcaccgacggcccctgcaaggtgcccgcccagatggccgtggacatgcccaccgccacccccgtgggcaggctgatcaccgccaaccccgtgatcaccgagagcaccgagaacagcaagatgaccctggagctggacccccccttcggcgacagctacatcgtgatcggcgtgggcgagaagaagatcacccaccactggcacaggagcggcagcaccatcggcaag

SEQ ID NO: 53 - DNA, 324 bp, Artificial Sequence
  source: /note=“Description of Artificial Sequence Synthetic polynucleotide”
ggcgtgagctacagcctgtgcaccgccgccttcaccttcgccgccgcccccgccgagaccctgcacggcaccgtggccgtggaggtgcagtacgccggcaccgacggcccctgcaaggtgcccgcccagatggccgtggacatggaggccgccacccccgtgggcaggctgatcaccgccaaccccgtgatcaccgagagcaccgagaacagcaagatggccctggagctggacccccccttcggcgacagctacatcgtgatcggcgtgggcgagaagaagatcacccaccactggcacaggagcggcagcaccatcggcaag

SEQ ID NO: 54 - DNA, 324 bp, Artificial Sequence
  source: /note=“Description of Artificial Sequence Synthetic polynucleotide”
ggcgtgagctacagcctgtgcaccgccgccttcaccttcaccaagatccccgccgagaccctgcacggcaccgtgaccgtggaggtgcagtacgccggcaccgacggcccctgcaaggtgcccgcccagatggccgtggacatgcagaccctgacccccgtgggcaggctgatcaccgccaaccccgtgatcaccgagagcaccgagaacagcaagatgatgctggagctggacccccccttcggcgacagctacatcgtgatcggcgtgggcgagaagaagatcacccaccactggcacaggagcggcagcaccatcggcaag

SEQ ID NO: 55 - protein, 108 aa, Zika virus; identical to SEQ ID NO: 16

SEQ ID NO: 56 - DNA, 324 bp, Zika virus; identical to SEQ ID NO: 17

SEQ ID NO: 57 - protein, 108 aa, Artificial Sequence
  source: /note=“Description of Artificial Sequence Synthetic polypeptide”
GVSYSLCTAAFTFAKTPAETLHGTVTVEVQYAGTDGPCKVPAQMAVDMAALAPVGRLITANPVITESTENSKMVLELDPPFGDSYIVIGVGEKKITHHWHRSGSTIGK

SEQ ID NO: 58 - DNA, 324 bp, Artificial Sequence
  source: /note=“Description of Artificial Sequence Synthetic polynucleotide”
ggtgtgagctatagcctgtgcaccgccgccttcaccttcgctaaaactccggccgaaaccctgcatggcaccgtgactgtggaagtgcagtatgccggtaccgatggtccgtgcaaagttccggcccagatggccgtggatatggcagctctggctccggtgggtcgcctgattaccgccaatccggtgattaccgagagcaccgagaacagcaaaatggtgctggaactggatccgccgttcggtgatagctatatcgtgattggcgtgggcgagaagaagatcacccatcattggcatcgcagcggtagcaccatcggcaaa

SEQ ID NO: 59 - protein, 108 aa, Artificial Sequence
  source: /note=“Description of Artificial Sequence Synthetic polypeptide”
GVSYSLCTAAFTFTAIPAETLHGTVTVEVQYAGTDGPCKVPAQMAVDMQALAPVGRLITANPVITESTENSKMVLELDPPFGDSYIVIGVGEKKITHHWHRSGSTIGK

SEQ ID NO: 60 - DNA, 324 bp, Artificial Sequence
  source: /note=“Description of Artificial Sequence Synthetic polynucleotide”
ggtgtgagctatagcctgtgcaccgccgccttcaccttcactgcaattccggccgaaaccctgcatggcaccgtgactgtggaagtgcagtatgccggtaccgatggtccgtgcaaagttccggcccagatggccgtggatatgcaagctctggctccggtgggtcgcctgattaccgccaatccggtgattaccgagagcaccgagaacagcaaaatggtgctggaactggatccgccgttcggtgatagctatatcgtgattggcgtgggcgagaagaagatcacccatcattggcatcgcagcggtagcaccatcggcaaa

SEQ ID NO: 61 - protein, 108 aa, Artificial Sequence
  source: /note=“Description of Artificial Sequence Synthetic polypeptide”
GVSYSLCTAAFTFTEVPAETLHGTVTVEVQYAGTDGPCKVPAQMAVDMETLAPVGRLITANPVITESTENSKMMLELDPPFGDSYIVIGVGEKKITHHWHRSGSTIGK

SEQ ID NO: 62 - DNA, 324 bp, Artificial Sequence
  source: /note=“Description of Artificial Sequence Synthetic polynucleotide”
ggtgtgagctatagcctgtgcaccgccgccttcaccttcactgaagttccggccgaaaccctgcatggcaccgtgactgtggaagtgcagtatgccggtaccgatggtccgtgcaaagttccggcccagatggccgtggatatggaaactctggctccggtgggtcgcctgattaccgccaatccggtgattaccgagagcaccgagaacagcaaaatgatgctggaactggatccgccgttcggtgatagctatatcgtgattggcgtgggcgagaagaagatcacccatcattggcatcgcagcggtagcaccatcggcaaa

SEQ ID NO: 63 - protein, 108 aa, Artificial Sequence
  source: /note=“Description of Artificial Sequence Synthetic polypeptide”
GVSYSLCTAAFTFTEAPAETLHGTVTVAVQYAGTDGPCKVPAQMAVDMETLAPVGRLITANPVITESTENSKMVLELDPPFGDSYIVIGVGEKKITHHWHRSGSTIGK

SEQ ID NO: 64 - DNA, 324 bp, Artificial Sequence
  source: /note=“Description of Artificial Sequence Synthetic polynucleotide”
ggtgtgagctatagcctgtgcaccgccgccttcaccttcactgaagctccggccgaaaccctgcatggcaccgtgactgtggcagtgcagtatgccggtaccgatggtccgtgcaaagttccggcccagatggccgtggatatggaaactctggctccggtgggtcgcctgattaccgccaatccggtgattaccgagagcaccgagaacagcaaaatggtgctggaactggatccgccgttcggtgatagctatatcgtgattggcgtgggcgagaagaagatcacccatcattggcatcgcagcggtagcaccatcggcaaa

SEQ ID NO: 65 - protein, 18 aa, Artificial Sequence
  source: /note=“Description of Artificial Sequence Synthetic peptide”
GSGSMAHIVMVDAYKPTK

SEQ ID NO: 66 - DNA, 54 bp, Artificial Sequence
  source: /note=“Description of Artificial Sequence Synthetic oligonucleotide”
ggttctggttctatggctcacatcgttatggttgacgcttacaaaccgaccaaa

SEQ ID NO: 67 - protein, 126 aa, Dengue virus 4
MPMHDYKDDDDKGSGMSYTMCSGKFSIDKEMAETQHGTTVVKVKYEGAGAPCKVPIEIRDVNKEKVVGRIISSTPFAENTNSVTNIELEPPFGDSYIVIGVGDSALTLHWFRKGSSIGKTCGRPSG

SEQ ID NO: 68 - DNA, 380 bp, Dengue virus 4
aatgcctatgcatgactacaaggatgatgatgacaaaggcagcggcatgagctacaccatgtgcagcggcaaattcagcatcgacaaggagatggccgaaacccagcatggcaccaccgtggtgaaagtgaaatacgaaggcgcaggtgccccgtgcaaagtgccgatcgaaatccgcgatgtgaacaaggagaaggtggtgggtcgcattatcagcagcaccccgttcgccgaaaacaccaacagcgtgaccaacatcgaactggaaccgccgtttggcgatagctatatcgtgattggcgtgggcgatagcgccctgaccctgcattggttccgcaaaggcagcagcattggtaaaacatgcggccggccctctggtt

SEQ ID NO: 69 - protein, 126 aa, Artificial Sequence
  source: /note=“Description of Artificial Sequence Synthetic polypeptide”
MPMHDYKDDDDKGSGMSAAMCSGKFSIDKEMAETEAGTTVVKVKYEGAGAPCKVPIEIRDVNKEKVVGRIISSTPFAENTNSVTNIELEPPFGDSYIVIGVGDSALTLHWFREGSSIGKTCGRPSG

SEQ ID NO: 70 - DNA, 380 bp, Artificial Sequence
  source: /note=“Description of Artificial Sequence Synthetic polynucleotide”
aatgcctatgcatgactacaaggatgatgatgacaaaggcagcggcatgagcgctgctatgtgcagcggcaaattcagcatcgacaaggagatggccgaaactgaagctggcaccaccgtggtgaaagtgaaatacgaaggcgcaggtgccccgtgcaaagtgccgatcgaaatccgcgatgtgaacaaggagaaggtggtgggtcgcattatcagcagcaccccgttcgccgaaaacaccaacagcgtgaccaacatcgaactggaaccgccgtttggcgatagctatatcgtgattggcgtgggcgatagcgccctgaccctgcattggttccgcgaaggcagcagcattggtaaaacatgcggccggccctctggtt

SEQ ID NO: 71 - protein, 126 aa, Artificial Sequence
  source: /note=“Description of Artificial Sequence Synthetic polypeptide”
MPMHDYKDDDDKGSGMSAAMCSGKFSIDKEMAETEHGTTVVKVKYEGAGAPCKVPIEIRDVNKEKVVGRIISSTPFAENTNSVTNIELEPPFGDSYIVIGVGDSALTLHWFRKGSSIGKTCGRPSG

SEQ ID NO: 72 - DNA, 380 bp, Artificial Sequence
  source: /note=“Description of Artificial Sequence Synthetic polynucleotide”
aatgcctatgcatgactacaaggatgatgatgacaaaggcagcggcatgagcgctgctatgtgcagcggcaaattcagcatcgacaaggagatggccgaaactgaacatggcaccaccgtggtgaaagtgaaatacgaaggcgcaggtgccccgtgcaaagtgccgatcgaaatccgcgatgtgaacaaggagaaggtggtgggtcgcattatcagcagcaccccgttcgccgaaaacaccaacagcgtgaccaacatcgaactggaaccgccgtttggcgatagctatatcgtgattggcgtgggcgatagcgccctgaccctgcattggttccgcaaaggcagcagcattggtaaaacatgcggccggccctctggtt

SEQ ID NO: 73 - protein, 126 aa, Artificial Sequence
  source: /note=“Description of Artificial Sequence Synthetic polypeptide”
MPMHDYKDDDDKGSGMSDAMCSGKFSIDKEMAATEHGTTVVKVKYEGAGAPCKVPIEIRDATTKAVVGGIISSTPFAENTNSVTNIELEPPFGDSYIVIGVGDSALTLHWFREGSSIGKTCGRPSG

SEQ ID NO: 74 - DNA, 380 bp, Artificial Sequence
  source: /note=“Description of Artificial Sequence Synthetic polynucleotide”
aatgcctatgcatgactacaaggatgatgatgacaaaggcagcggcatgagctacaccatgtgcagcggcaaattcagcatcgacaaggagatggccgcaactgaacatggcaccaccgtggtgaaagtgaaatacgaaggcgcaggtgccccgtgcaaagtgccgatcgaaatccgcgatgctaccacaaaagcagtggtgggtggtattatcagcagcaccccgttcgccgaaaacaccaacagcgtgaccaacatcgaactggaaccgccgtttggcgatagctatatcgtgattggcgtgggcgatagcgccctgaccctgcattggttccgcgaaggcagcagcattggtaaaacatgcggccggccctctggtt

SEQ ID NO: 75 - protein, 126 aa, Artificial Sequence
  source: /note=“Description of Artificial Sequence Synthetic polypeptide”
MPMHDYKDDDDKGSGMSDAMCSGKFSIDKEMAETADGTTVVKVKYEGAGAPCKVPIEIRDVDTAAVVGRIISSTPFAENTNSVTNIELEPPFGDSYIVIGVGDSALTLHWFRAGSSIGKTCGRPSG

SEQ ID NO: 76 - DNA, 380 bp, Artificial Sequence
  source: /note=“Description of Artificial Sequence Synthetic polynucleotide”
aatgcctatgcatgactacaaggatgatgatgacaaaggcagcggcatgagcgatgctatgtgcagcggcaaattcagcatcgacaaggagatggccgaaactgcagatggcaccaccgtggtgaaagtgaaatacgaaggcgcaggtgccccgtgcaaagtgccgatcgaaatccgtgatgttgacacagcagcagtggtgggtcgtattatcagcagcaccccgttcgccgaaaacaccaacagcgtgaccaacatcgaactggaaccgccgtttggcgatagctatatcgtgattggcgtgggcgatagcgccctgaccctgcattggttccgcgcaggcagcagcattggtaaaacatgcggccggccctctggtt

SEQ ID NO: 77 - protein, 126 aa, Artificial Sequence
  source: /note=“Description of Artificial Sequence Synthetic polypeptide”
MPMHDYKDDDDKGSGMSDAMCSGKFSIDKDMAETEDGTTVVKVKYKRACAPCKVPIEIRDVNKDNVVGRIISSTPFAENTNSVTNIELEPPFGDSYIVIGVGDSALTLHWFREGSSIGKTCGRPSG

SEQ ID NO: 78 - DNA, 380 bp, Artificial Sequence
  source: /note=“Description of Artificial Sequence Synthetic polynucleotide”
aatgcctatgcatgactacaaggatgatgatgacaaaggcagcggcatgagcgatgctatgtgcagcggcaaattcagcatcgacaaggatatggccgaaactgaagatggcaccaccgtggtgaaagtgaaatacaaacgcgcatgtgccccgtgcaaagtgccgatcgaaatccgcaatgtgaacaaggataatgtggtgggtcgcattatcaacaccaccccgttcgccgaaaacaccaacagcgtgaccaacatcgaactggaaccgccgtttggcgatagctatatcgtgattggcgtgggcgatagcgccctgaccctgcattggttccgcaaaggcagcagcattggtaaaacatgcggccggccctctggtt

SEQ ID NO: 79 - protein, 126 aa, Artificial Sequence
  source: /note=“Description of Artificial Sequence Synthetic polypeptide”
MPMHDYKDDDDKGSGMSDAMCSGKFSIDKEMAETPDGTTVVKVKYEGAGAPCKVPIEIRDVNKEKVVGRIISSTPFAENTNSVTNIELEPPFGDSYIVIGVGDSALTLHWFREGSSIGKTCGRPSG

SEQ ID NO: 80 - DNA, 380 bp, Artificial Sequence
  source: /note=“Description of Artificial Sequence Synthetic polynucleotide”
aatgcctatgcatgactacaaggatgatgatgacaaaggcagcggcatgagcgatgctatgtgcagcggcaaattcagcatcgacaaggagatggccgaaactccagatggcaccaccgtggtgaaagtgaaatacgaaggcgcaggtgccccgtgcaaagtgccgatcgaaatccgcgatgtgaacaaggagaaggtggtgggtcgcattatcagcagcaccccgttcgccgaaaacaccaacagcgtgaccaacatcgaactggaaccgccgtttggcgatagctatatcgtgattggcgtgggcgatagcgccctgaccctgcattggttccgcgaaggcagcagcattggtaaaacatgcggccggccctctggtt

SEQ ID NO: 81 - protein, 126 aa, Artificial Sequence
  source: /note=“Description of Artificial Sequence Synthetic polypeptide”
MPMHDYKDDDDKGSGMSSAMCTGKFSINKEMAATEAGTTVVKVKYEGAGAPCKVPIEIRDADKAKVVGGIISSTPFAENTNSVTNIELEPPFGDSYIVIGVGDSALTLHWFREGSSIGKTCGRPSG

SEQ ID NO: 82 - DNA, 380 bp, Artificial Sequence
  source: /note=“Description of Artificial Sequence Synthetic polynucleotide”
aatgcctatgcatgactacaaggatgatgatgacaaaggcagcggcatgagctctgctatgtgcaccggcaaattcagcatcaacaaggagatggccgcaactgaagctggcaccaccgtggtgaaagtgaaatacgaaggcgcaggtgccccgtgcaaagtgccgatcgaaatccgtgatgctgacaaagcaaaagtggtgggtggtattatcagcagcaccccgttcgccgaaaacaccaacagcgtgaccaacatcgaactggaaccgccgtttggcgatagctatatcgtgattggcgtgggcgatagcgccctgaccctgcattggttccgcgaaggcagcagcattggtaaaacatgcggccggccctctggtt

SEQ ID NO: 83 - protein, 126 aa, Artificial Sequence
  source: /note=“Description of Artificial Sequence Synthetic polypeptide”
MPMHDYKDDDDKGSGMSYAMCSGKFSIDKEMAAAADGTTVVKVKYEGAGAPCKVPIEIRDAAKAAVVGGIISSTPFAENTNSVTNIELEPPFGDSYIVIGVGDSALTLHWFREGSSIGKTCGRPSG

SEQ ID NO: 84 - DNA, 380 bp, Artificial Sequence
  source: /note=“Description of Artificial Sequence Synthetic polynucleotide”
aatgcctatgcatgactacaaggatgatgatgacaaaggcagcggcatgagctatgctatgtgcagcggcaaattcagcatcgacaaggagatggccgcagctgcagatggcaccaccgtggtgaaagtgaaatacgaaggcgcaggtgccccgtgcaaagtgccgatcgaaatccgtgatgctgccaaagcagcagtggtgggtggtattatcagcagcaccccgttcgccgaaaacaccaacagcgtgaccaacatcgaactggaaccgccgtttggcgatagctatatcgtgattggcgtgggcgatagcgccctgaccctgcattggttccgcgaaggcagcagcattggtaaaacatgcggccggccctctggtt

SEQ ID NO: 85 - protein, 126 aa, Artificial Sequence
  source: /note=“Description of Artificial Sequence Synthetic polypeptide”
MPMHDYKDDDDKGSGMSYTMCSGKFSIDKEMAEIPHGTTVVKVKYEGAGAPCKVPIEIRDATKAKVVGPIISSTPFAENTNSVTNIELEPPFGDSYIVIGVGDSALTLHWFREGSSIGKTCGRPSG

SEQ ID NO: 86 - DNA, 380 bp, Artificial Sequence
  source: /note=“Description of Artificial Sequence Synthetic polynucleotide”
aatgcctatgcatgactacaaggatgatgatgacaaaggcagcggcatgagctacaccatgtgcagcggcaaattcagcatcgacaaggagatggccgaaactccacatggcaccaccgtggtgaaagtgaaatacgaaggcgcaggtgccccgtgcaaagtgccgatcgaaatccgtgatgctaccaaagcaaaagtggtgggtcctattatcagcagcaccccgttcgccgaaaacaccaacagcgtgaccaacatcgaactggaaccgccgtttggcgatagctatatcgtgattggcgtgggcgatagcgccctgaccctgcattggttccgcgaaggcagcagcattggtaaaacatgcggccggccctctggtt

SEQ ID NO: 87 - protein, 4 aa, Dengue virus 4
ETQH

SEQ ID NO: 88 - protein, 4 aa, Artificial Sequence
  source: /note=“Description of Artificial Sequence Synthetic peptide”
ETEA

SEQ ID NO: 89 - protein, 4 aa, Artificial Sequence
  source: /note=“Description of Artificial Sequence Synthetic peptide”
ETEH

SEQ ID NO: 90 - protein, 4 aa, Artificial Sequence
  source: /note=“Description of Artificial Sequence Synthetic peptide”
ETPD

SEQ ID NO: 91 - protein, 4 aa, Artificial Sequence
  source: /note=“Description of Artificial Sequence Synthetic peptide”
ATEH

SEQ ID NO: 92 - protein, 4 aa, Artificial Sequence
  source: /note=“Description of Artificial Sequence Synthetic peptide”
ATPH

SEQ ID NO: 93 - protein, 4 aa, Artificial Sequence
  source: /note=“Description of Artificial Sequence Synthetic peptide”
ETED

SEQ ID NO: 94 - protein, 4 aa, Artificial Sequence
  source: /note=“Description of Artificial Sequence Synthetic peptide”
ATEA

SEQ ID NO: 95 - protein, 4 aa, Artificial Sequence
  source: /note=“Description of Artificial Sequence Synthetic peptide”
ATHD

SEQ ID NO: 96 - protein, 4 aa, Artificial Sequence
  source: /note=“Description of Artificial Sequence Synthetic peptide”
EAED

SEQ ID NO: 97 - protein, 5 aa, Artificial Sequence
  source: /note=“Description of Artificial Sequence Synthetic peptide”
VNKEK

SEQ ID NO: 98 - protein, 5 aa, Artificial Sequence
  source: /note=“Description of Artificial Sequence Synthetic peptide”
ADTEK

SEQ ID NO: 99 - protein, 5 aa, Artificial Sequence
  source: /note=“Description of Artificial Sequence Synthetic peptide”
ATTEA

SEQ ID NO: 100 - protein, 5 aa, Artificial Sequence
  source: /note=“Description of Artificial Sequence Synthetic peptide”
AAKEK

SEQ ID NO: 101 - protein, 5 aa, Artificial Sequence
  source: /note=“Description of Artificial Sequence Synthetic peptide”
ADKAK

SEQ ID NO: 102 - protein, 5 aa, Artificial Sequence
  source: /note=“Description of Artificial Sequence Synthetic peptide”
ATKEK

SEQ ID NO: 103 - protein, 5 aa, Artificial Sequence
  source: /note=“Description of Artificial Sequence Synthetic peptide”
ADEET

SEQ ID NO: 104 - protein, 5 aa, Dengue virus 4
VNKEK

(19) United States
(12) Patent Application Publication
(10) Pub. No.: US 2022/0372079 A1
(43) Pub. Date: Nov. 24, 2022

(54) RESURFACED DENGUE VIRUS AND ZIKI VIRUS GLYCOPROTEIN E DIII VARIANTS AND USES THEREOF

(71) Applicants: Jonathan R. Lai, Dobbs Ferry, NY (US); Julia Frei, Brugg (CH); Olivia Vergnolle, Jackson Heights, NY (US); Elisabeth Nyakatura, Jersey City, NY (US); George Georgiev, Bronx, NY (US); Ariel Wirchnianski, Bronx, NY (US)

(72) Inventors: Jonathan R. Lai, Dobbs Ferry, NY (US); Julia Frei, Brugg (CH); Olivia Vergnolle, Jackson Heights, NY (US); Elisabeth Nyakatura, Jersey City, NY (US); George Georgiev, Bronx, NY (US); Ariel Wirchnianski, Bronx, NY (US)

(21) Appl. No.: 17/621,513

(22) PCT Filed: Jun. 24, 2020
(86) PCT No.: PCT/US20/39403

§ 371 Date: Dec. 21, 2021

Related U.S. Application Data
(60) Provisional application No. 62/865,627, filed on Jun. 24, 2019.

(51) Int. Cl. C07K 14/005; C12N 7/00; A61K 39/12; A61P 31/14

(52) U.S. Cl. CPC C07K 14/005; C12N 7/00; A61K 39/12; A61P 31/14; C12N 2770/24122; C12N 2770/24134; C12N 2770/24171

(57) ABSTRACT

Specific resurfaced Dengue virus glycoprotein subunit E DIII variants and their uses in preventing and treating Dengue virus infection are disclosed. Provided herein are specific resurfaced Zika virus glycoprotein subunit E DIII variants and their uses in preventing and treating Zika virus infection. Multivalent vaccines comprising such resurfaced EDIII variants are also described, in addition to nanoparticle conjugated resurfaced EDIII variants.

### CROSS REFERENCE TO RELATED APPLICATIONS

This application claims priority to U.S. Provisional Application No: 62/865,627, filed Jun. 24, 2019, the contents of which are incorporated herein by reference in their entirety.

### STATEMENT OF GOVERNMENT SUPPORT

This invention was made with government support under grant numbers R21-AI128090 awarded by the National Institutes of Health. The government has certain rights in the invention.

### BACKGROUND OF THE INVENTION

Dengue virus is the leading arthropod-transmitted viral disease in the world with approximately 390 million human infections per year (Bhatt, S., et al. (2013) Nature 496, 504-507). Nearly 3.6 billion people live in at risk areas for infection, and the global distribution of the two mosquito species that carry the virus (Aedes aegypti and Aedes albopictus) is expanding beyond tropical regions and reaches as far north as New York in North America (Kraemer, M. U., et al. (2015) eLife 4, e08347). Primary infection by one of the four Dengue virus serotypes (DENV1-4) typically causes a significant but self-limiting febrile illness, whereas secondary infections can lead to severe disease characterized by hemorrhagic fever and shock syndrome (Severe Dengue or Dengue Hemorrhagic Fever (DHF) or Dengue Shock Syndrome (DSS)). These latter syndromes occur in a minor fraction (1% or less) of secondary infections but lead to hospitalization and, in some cases, death. DHF and DSS are thought to arise from a process known as antibody-dependent enhancement (ADE) of infection. In an increasingly accepted model, ADE is caused by antibodies elicited during the course of primary infection that may be potently neutralizing against the primary infection serotype, but also have some cross-reactivity or weak neutralization potential against other serotypes (Guzman, M. G., et al. (2013) Archives of virology 158, 1445-1459). During secondary infection by a heterologous DENV serotype, these antibodies promote uptake and infection of the un-neutralized virus in Fc-γ receptor (FcγR) expressing cells, ultimately increasing viremia. This leads to greater levels of pro-inflammatory cytokines (e.g., IL-1β, TNF-α, IL-6, IFN-γ) and the viral NS1 protein in serum, both of which compromise junctional integrity of capillary endothelial cells (Guzman, M. G., et al. (2013) Archives of virology 158, 1445-1459). Structural proteins encoded by the DENY genome diverge by as much as 40% in amino acid sequence among the four serotypes, and within each of the serotypes, individual genotypes vary by ˜3%. Thus, a critical objective for Dengue virus vaccine design is to elicit a broadly neutralizing antibody response against all four serotypes, since weakly cross-reactive antibodies may actually increase the risk of ADE.

Three Dengue vaccine candidates are in clinical development, all of which consist of tetravalent mixtures of attenuated or chimeric viruses. In recently published phase III trials, Sanofi's Dengvaxia®, a tetravalent mixture of yellow-fever virus vector containing DENV1-4 glycoprotein, provided only partial efficacy (<70%) in seropositive cases, and was not effective at all for naïve individuals (Hadinegoro, S. R., et al. (2015) The NEJM 373, 1195-1206). Nonetheless, Dengvaxia® was recently approved for use in Mexico, the Philippines, Brazil and several other countries in children over the age of 9 who are presumably already flavivirus immune. Two other candidate vaccines are in moving into phase III trials (DENVax, Takeda; and TV003/TV005, NIAID); yet, both also elicited incomplete levels of neutralizing antibody responses (Osorio, J. E., et al. (2014) The Lancet. Infectious diseases 14, 830-838; Kirkpatrick, B. D., et al. (2015) The J of infectious diseases 212, 702-710). Nonetheless, all three vaccines consist of mixtures of live attenuated viruses. Vaccines consisting of fewer components that avoid live virus strategies with their possible side effects are desirable. Therefore, there is significant need for the development of alternative vaccine platforms for use either as next-generation primary vaccines, or as boosting agents to improve the efficacy of existing live virus vaccines.

The emergence of Zika virus (“ZIKV”), another flavivirus, in the Americas has complicated flavivirus vaccine development. Most infections by ZIKV are relatively mild, but severe neurological manifestations, such as Guillain-Barre syndrome and microcephaly in newborn infants have been associated with infection. The structure of the ZIKV virion is similar to that of other flaviviruses, and a number of mAbs targeting DENY E also bind to ZIKV E (Zhao, H. et al. (2016) Cell 166, 1016-1027; Sirohi, D. et al. (2016) Science (New York, N.Y.) 352, 467-470; Stettler, K. et al. (2016) Science (New York, N.Y.) 353, 823-826). In fact, cross-reactive mAbs, as well as sera from DENY patients, can induce ADE of ZIKV in vitro (Dejnirattisai, W. et al. (2016) Nat Immunol 17, 1102-1108). ZIKV is likely to become endemic in regions where its primary vector (Aedes aegypti) is prevalent.

Therefore, a critical objective for future flavivirus vaccine development will be to devise strategies that afford cross-protection from DENY and ZIKV, but do not stimulate ADE across viral species or serotypes. Therefore, there is a need to develop ZIKV immunogens based on EDIII for use alone, or in DENV/ZIKV multivalent vaccines.

The present invention addresses the need for improved methods for preventing and treating Dengue virus infections by providing resurfaced Dengue virus 4 glycoprotein subunit E domain III (EDIII)-based (“rsD4DIIIs” variants) vaccines. Also provided herein are methods for preventing and treating Zika virus infections by providing resurfaced Zika virus glycoprotein EDIII-based (“rsZDIIIs” variants) vaccines. Such rsD4DIIIs and rsZDIIIs variants can be used alone as vaccines, or in combination to generate multivalent vaccines.

### SUMMARY OF THE INVENTION

Provided herein are specific resurfaced Dengue virus 4 glycoprotein subunit E DIII variants (“rsD4DIIIs”) and their uses in preventing and treating Dengue virus infection. Also provided are specific resurfaced Zika virus glycoprotein subunit E DIII variants (“rsZDIIIs”) and their uses in preventing and treating Zika virus infection. Such rsD4DIIIs can to mix with other rsD4DIIIs for broad neutralization. Such rsD4DIIIs can to mix with rsZDIIIs to create DENV/ZIKV multivalent vaccines. Such rsD4DIIIs or rsZDIIIs, or combinations thereof, can be complexed, conjugated, or coupled with nanoparticles to generate nanoparticles containing rsD4DIIIs and/or rsZDIIIs for multivalent presentation.

One aspect of the invention relates to a resurfaced Dengue virus-4 glycoprotein subunit E DIII variant comprising variant rsD4DIII-1 (SEQ ID NO:3) or (SEQ ID NO:69), variant rsD4DIII-2 (SEQ ID NO:4) or (SEQ ID NO:71), variant rsD4DIII-3 (SEQ ID NO:5), variant rsD4DIII-4 (SEQ ID NO:6) or (SEQ ID NO:73), variant rsD4DIII-5 (SEQ ID NO:7), variant rsD4DIII-7 (SEQ ID NO:75), variant rsD4DIII-8 (SEQ ID NO:8) or (SEQ ID NO:77), variant rsD4DIII-9 (SEQ ID NO:9) or (SEQ ID NO:79), variant rsD4DIII-10 (SEQ ID NO:10) or (SEQ ID NO:81), variant rsD4DIII-11 (SEQ ID NO:83), variant rsD4DIII-12 (SEQ ID NO:11), variant rsD4DIII-13 (SEQ ID NO:12), variant rsD4DIII-14 (SEQ ID NO:13) variant rsD4DIII-18 (SEQ ID NO:85).

In some embodiments, the variant consists of rsD4DIII-1 (SEQ ID NO:3) or (SEQ ID NO:69).

In some embodiments, the variant consists of rsD4DIII-2 (SEQ ID NO:4) or (SEQ ID NO:71).

In some embodiments, the variant consists of rsD4DIII-3 (SEQ ID NO:5).

In some embodiments, the variant consists of rsD4DIII-4 (SEQ ID NO:6).

In some embodiments, the variant consists of rsD4DIII-5 (SEQ ID NO:7).

In some embodiments, the variant consists of rsD4DIII-7 (SEQ ID NO:75).

In some embodiments, the variant consists of rsD4DIII-8 (SEQ ID NO:8) or (SEQ ID NO:77).

In some embodiments, the variant consists of rsD4DIII-9 (SEQ ID NO:9) or (SEQ ID NO:79).

In some embodiments, the variant consists of rsD4DIII-10 (SEQ ID NO:10) or (SEQ ID NO:81).

In some embodiments, the variant consists of rsD4DIII-11 (SEQ ID NO:83).

In some embodiments, the variant consists of rsD4DIII-12 (SEQ ID NO:11).

In some embodiments, the variant consists of rsD4DIII-13 (SEQ ID NO:12).

In some embodiments, the variant consists of rsD4DIII-14 (SEQ ID NO:13).

In some embodiments, the variant consists of rsD4DIII-18 (SEQ ID NO:85).

In some embodiments, the variant is encoded by the nucleic acid set forth in SEQ ID NO:32 or SEQ ID NO: 70.

In some embodiments, the variant is encoded by the nucleic acid set forth in SEQ ID NO:33 or SEQ ID NO: 71.

In some embodiments, the variant is encoded by the nucleic acid set forth in SEQ ID NO:34.

In some embodiments, the variant is encoded by the nucleic acid set forth in SEQ ID NO:35 or SEQ ID NO: 73.

In some embodiments, the variant is encoded by the nucleic acid set forth in SEQ ID NO:36.

In some embodiments, the variant is encoded by the nucleic acid set forth in SEQ ID NO:76.

In some embodiments, the variant is encoded by the nucleic acid set forth in SEQ ID NO:37 or SEQ ID NO: 78. 100311 In some embodiments, the variant is encoded by the nucleic acid set forth in SEQ ID NO:38 or SEQ ID NO: 80.

In some embodiments, the variant is encoded by the nucleic acid set forth in SEQ ID NO:39 or SEQ ID NO: 82.

In some embodiments, the variant is encoded by the nucleic acid set forth in SEQ ID NO:84.

In some embodiments, the variant is encoded by the nucleic acid set forth in SEQ ID NO:40.

In some embodiments, the variant is encoded by the nucleic acid set forth in SEQ ID NO:41.

In some embodiments, the variant is encoded by the nucleic acid set forth in SEQ ID NO:42.

In some embodiments, the variant is encoded by the nucleic acid set forth in SEQ ID NO:86.

Another aspect of the invention relates to a resurfaced Zika virus glycoprotein subunit E DIII variant comprising variant rsZDIII-1.20 (SEQ ID NO:18) or (SEQ ID NO:57), variant rsZDIII-1.39 (SEQ ID NO:19) or (SEQ ID NO:59), variant rsZDIII-1.48 (SEQ ID NO:20), variant rsZDIII-1.69 (SEQ ID NO:21), variant rsZDIII-1.74 (SEQ ID NO:22), variant rsZDIII-2.16 (SEQ ID NO:23) or (SEQ ID NO:61), variant rsZDIII-2.39 (SEQ ID NO:24) or (SEQ ID NO:63), variant rsZDIII-2.50 (SEQ ID NO:25), variant rsZDIII-1.8 (SEQ ID NO:26), variant rsZDIII-1.25 (SEQ ID NO:27), or variant rsZDIII-1.27 (SEQ ID NO:28).

In some embodiments, the variant consists of rsZDIII-1.20 (SEQ ID NO:18) or (SEQ ID NO:57).

In some embodiments, the variant consists of rsZDIII-1.39 (SEQ ID NO:19) or (SEQ ID NO:59).

In some embodiments, the variant consists of rsZDIII-1.48 (SEQ ID NO:20).

In some embodiments, the variant consists of rsZDIII-1.69 (SEQ ID NO:21).

In some embodiments, the variant consists of rsZDIII-1.74 (SEQ ID NO:22).

In some embodiments, the variant consists of rsZDIII-2.16 (SEQ ID NO:23) or (SEQ ID NO:61).

In some embodiments, the variant consists of rsZDIII-2.39 (SEQ ID NO:24) or (SEQ ID NO:63).

In some embodiments, the variant consists of rsZDIII-2.50 (SEQ ID NO:25).

In some embodiments, the variant consists of rsZDIII-1.8 (SEQ ID NO:26).

In some embodiments, the variant consists of rsZDIII-1.25 (SEQ ID NO:27).

In some embodiments, the variant consists of rsZDIII-1.27 (SEQ ID NO:28).

In some embodiments, the variant is encoded by the nucleic acid set forth in SEQ ID NO:44 or SEQ ID NO: 58.

In some embodiments, the variant is encoded by the nucleic acid set forth in SEQ ID NO:45 or SEQ ID NO: 60.

In some embodiments, the variant is encoded by the nucleic acid set forth in SEQ ID NO:46.

In some embodiments, the variant is encoded by the nucleic acid set forth in SEQ ID NO:47.

In some embodiments, the variant is encoded by the nucleic acid set forth in SEQ ID NO:48 or SEQ ID NO: 62.

In some embodiments, the variant is encoded by the nucleic acid set forth in SEQ ID NO:49 or SEQ ID NO: 64.

In some embodiments, the variant is encoded by the nucleic acid set forth in SEQ ID NO:50.

In some embodiments, the variant is encoded by the nucleic acid set forth in SEQ ID NO:51.

In some embodiments, the variant is encoded by the nucleic acid set forth in SEQ ID NO:52

In some embodiments, the variant is encoded by the nucleic acid set forth in SEQ ID NO:53

In some embodiments, the variant is encoded by the nucleic acid set forth in SEQ ID NO:54

Another aspect of the invention relates to a dimer or oligomer comprising any of the aforementioned variants.

Another aspect of the invention relates to a virion of an isolated, recombinant Dengue virus comprising any of the aforementioned variants or any of the aforementioned dimers or oligomers.

Another aspect of the invention relates to a Dengue virus vaccine composition comprising any of the aforementioned variants, any of the aforementioned dimers or oligomers, or any of the aforementioned virions.

Another aspect of the invention relates to a virion of an isolated, recombinant Zika virus comprising any of the aforementioned variants, or the any of the aforementioned dimers or oligomers.

Another aspect of the invention relates to a Zika virus vaccine composition comprising any of the aforementioned variants, any of the aforementioned dimers or oligomers, or any of the aforementioned virions.

In some embodiments, the vaccine composition further comprises an immunological adjuvant.

In some embodiments, the vaccine composition is conjugated to at least one nanoparticle.

In some embodiments, the vaccine composition is conjugated to at least one nanoparticle, wherein said composition comprises any of the variants of claim 1-52 engineered with a C-terminus tag comprising SEQ ID NO: 65.

When used in the vaccination studies, any of the aforementioned variants are engineered with a C-term SpyTag sequence to facilitate aaLS nanoparticle conjugation. SpyTag Nucleotide Sequence: GGTTCTGGTTCTATGGCTCACATCGTTATGGTTGACGCTTACAAACCGACCAAA (SEQ ID NO: 66). SpyTag Amino acid Sequence: GSGSMAHIVMVDAYKPTK (SEQ ID NO: 65)

Another aspect of the invention relates to a method of eliciting an immune response in a subject comprising administering to the subject any of the aforementioned variants, any of the aforementioned dimers or oligomers, any of the aforementioned virions, or any of the aforementioned vaccines in an amount effective to elicit an immune response in a subject.

Another aspect of the invention relates to a method of vaccinating a subject for Dengue virus infection comprising administering to the subject any of the aforementioned variants, any of the aforementioned dimers or oligomers, any of the aforementioned virions, or any of the aforementioned vaccines in an amount effective to vaccinate a subject for Dengue virus.

Another aspect of the invention relates to a method of immunizing a subject against Dengue virus infection comprising administering to the subject any of the aforementioned variants, any of the aforementioned dimers or oligomers, any of the aforementioned virions, or any of the aforementioned vaccines in an amount effective to immunize a subject against Dengue virus.

Another aspect of the invention relates to a method of treating a Dengue virus infection in a subject or treating a disease caused by a Dengue virus infection in a subject comprising administering to the subject any of the aforementioned variants, any of the aforementioned dimers or oligomers, any of the aforementioned virions, or any of the aforementioned vaccines in an amount effective to treat a Dengue virus infection or treat a disease caused by a Dengue virus infection in a subject.

In some embodiments of the methods, the subject has one or more of Dengue Hemorrhagic Fever (DHF) and Dengue Shock Syndrome (DSS).

In some embodiments of the methods, the variant, dimer, oligomer, virion or vaccine is effective against all Dengue virus serotypes.

Another aspect of the invention relates to a method of vaccinating a subject for Zika virus infection comprising administering to the subject any of the aforementioned variants, any of the aforementioned dimers or oligomers, any of the aforementioned virions, or any of the aforementioned vaccines in an amount effective to vaccinate a subject for Zika virus.

Another aspect of the invention relates to a method of immunizing a subject against Zika virus infection comprising administering to the subject any of the aforementioned variants, any of the aforementioned dimers or oligomers, any of the aforementioned virions, or any of the aforementioned vaccines in an amount effective to immunize a subject against Zika virus.

Another aspect of the invention relates to a method of treating a Zika virus infection in a subject or treating a disease caused by a Zika virus infection in a subject comprising administering to the subject any of the aforementioned variants, any of the aforementioned dimers or oligomers, any of the aforementioned virions, or any of the aforementioned vaccines in an amount effective to treat a Zika virus infection or treat a disease caused by a Zika virus infection in a subject.

In some embodiments of the methods, the variant, dimer, oligomer, virion or vaccine is effective against all Zika virus serotypes.

### BRIEF DESCRIPTION OF THE DRAWINGS

FIG. 1, contains four panels, 1A-1D, depicting resurfacing of DENV4 EDIII (“rsD4DIII”). FIG. 1A shows that the rsD4DIII library design strategy involves randomization of the AB-loop (cyan spheres) and other surface exposed residues (orange spheres), while maintaining critical LR epitopes (red and blue spheres). FIG. 1B shows eleven representative sequences from a total of 15 selected clones. Substitutions to Ala indicated in red, and to other side chains in green. FIG. 1C shows phage ELISA reactivity profiles of WT DENV4 EDIII and resurfaced variants. FIG. 1D shows ELISA reactivity of DENV4-specific mAbs against WT DENV4 DIII and rsD4DIIIs.

FIG. 2, contains four panels, 2A-2D, depicting resurfacing of ZIKV EDIII (“rsZDIII”). FIG. 2A shows X-ray structure of ZV-67 bound to ZIKV EDIII (PDB ID 5VKG, Tharakaraman, K. et al. (2013) PNAS 110, E1555-1564). For resurfacing, residues in the ABDE-sheet and CC′-loop were randomized in restricted fashion (red spheres). FIG. 2B shows eleven representative sequences from a total of 18 selected clones. Substitutions to Ala indicated in red, and to other side chains in green. FIG. 2C shows phage ELISA reactivity profiles of WT ZIKV EDIII and resurfaced variants. FIG. 2D shows reactivity of ZIKV-specific mAbs against WT ZIKV EDIII and rsZDIIIs.

FIG. 3, contains two panels, 3A-3B, showing variability in the potency of serum antibody response to monomeric WT DENV2 EDIII and rsDIIIs. FIG. 3A depicts FRNT50 (top) and FRNT80 (bottom) values for individual sera from immunized mice against DENV1-3 (Kruskal-Wallis one-way ANOVA with Dunn's post-test (*P<0.05; **P<0.007; ***P<0.001)). FIG. 3B shows transfer of pooled WT DENV2 EDIII or rsDIII-Ala30 sera (days 60 and 90) to AG129 mice did not protect against lethal DENV2 (strain D2S20) challenge. The curves for WT DENV2 EDIII and rsDIII-Ala30 are directly on top of one another.

FIG. 4, contains six panels, 4A-4F, showing generation and characterization of rsD4DIII-decorated nanoparticles. Scheme (FIG. 4A) and gel (FIG. 4B) for conjugation of aaLS or hpFer nanoparticles using the Spycatcher/Spytag system. FIG. 4C and FIG. 4D show electron microscopy of aaLS-SpyC-rsD4DIII and hpFer-SpyC-rsD4DIII nanoparticles, respectively. FIG. 4E and FIG. 4F show both rsD4DIII-conjugated nanoparticles are reactive toward DV4-E88 (a DENV4-specific neutralizing antibody), whereas undecorated nanoparticles show no activity.

### DETAILED DESCRIPTION OF THE INVENTION

Dengue Virus

The mature, prefusion glycoprotein E exists as a head-to-tail dimer organized into rafts with icosahedral geometry on the viral particle (Kuhn, R. J., et al. (2002) Cell 108, 717-725; Modis, Y., et al. (2003) PNAS 100, 6986-6991). Each E subunit contains three domains, DI, DII, and DIII. DII contains the fusion loop that inserts into the host cell upon initiation of the fusion reaction in the endosome. DI acts as a rigid connector to DIII, which is anchored via the stem and C-terminal TM domain into the viral membrane. The post-fusion E structure is a trimer with the DIII domain and stem region significantly relocated relative to DI and DII, so as to bring the host and viral membranes into proximity to facilitate viral membrane fusion (Modis, Y., et al. (2004) Nature 427, 313-319). A host receptor has yet to be identified, but there is circumstantial evidence that interactions between cellular components and DIII initiate attachment and infection (Chiu, M. W., et al. (2003) Biochemical and biophysical res comm 309, 672-678; Watterson, D., et al. (2012) The J of Gen Virol 93, 72-82; Huerta, V., et al. (2008) Virus research 137, 225-234). Neutralizing antibodies arising during infection target a variety of epitopes on the E glycoprotein. Potent and cross-neutralizing antibodies appear to be directed toward either complex quaternary epitopes whose constituents involve portions of the E domains on adjacent dimer subunits (Dejnirattisai, W., et al. (2015) Nature immunology 16, 170-177; Rouvinski, A., et al. (2015) Nature 520, 109-113), or toward the lateral ridge on DIII formed by the A and G strands (Cockburn, J. J., et al. (2012) Structure 20, 303-314; Lok, S. M., et al. (2008) Nature structural & molecular biology 15, 312-317). One example of a DIII-specific broadly neutralizing antibody (bNAb) is the murine mAb 4E11 that potently neutralizes DENV1-3 and weakly neutralizes DENV4 (Cockburn, J. J., et al. (2012) Structure 20, 303-314) for the crystal structure of the DIII-4E11 complex). Recently, high-throughput mutagenesis (“combinatorial alanine scanning”) was used to quantify energetic contributions of contact residues on DIII from all four serotypes recognition feature for 4E11 (Frei, J. C., et al. (2015) Virology 485, 371-382).

Immunization of mice and non-human primates with recombinant DIII constructs (EDIIIs) leads to strong antibody responses, but these antibodies are poorly neutralizing or limited in breadth (Suzarte, E., et al. (2015) International immunology 27, 367-379; Gil, L., et al. (2015) Immunology and cell biology 93, 57-66; Izquierdo, A., et al. (2014) Archives of virology 159, 2597-2604; Garcia-Machorro, J., et al. (2013) Human vaccines & immunotherapeutics 9, 2326-2335; Li, X.-Q., et al. (2013) Journal of General Virology 94, 2191-2201; Chen, H. W., et al. (2013) Archives of virology 158, 1523-1531; Arora, U., et al (2013) Vaccine 31, 873-878; Zhao, H., et al. (2014) PloS one 9, e86573; Sukupolvi-Petty, S., et al. (2007) Journal of virology 81, 12816-12826; Midgley, C. M., et al. (2012) Journal of immunology (Baltimore, Md.: 1950) 188, 4971-4979; Valdes, I., et al. (2009) Vaccine 27, 995-1001). In mice, the immunodominant regions of DIII appear to be in the AB- and FG-loops; resulting monoclonal antibodies are either cross-reactive and non-neutralizing (AB-loop) or type-specific and variably neutralizing (FG-loop) (Sukupolvi-Petty, S., et al. (2007) Journal of virology 81, 12816-12826; Midgley, C. M., et al. (2012) Journal of immunology (Baltimore, Md.: 1950) 188, 4971-4979). Antibodies that target other domains or more complex epitopes predominate in the human response during the course of natural infection (Dejnirattisai, W., et al. (2015) Nature immunology 16, 170-177; Rouvinski, A., et al. (2015) Nature 520, 109-113; de Alwis, R., et al. (2012) Proceedings of the National Academy of Sciences of the United States of America 109, 7439-7444; Smith, S. A., et al. (2013) mBio 4, e00873-00813). Immunization of non-human primates with EDIII generates a high DIII-specific antibody titer (Gil, L., et al. (2015) Immunology and cell biology 93, 57-66; Chen, H. W., et al. (2013) Archives of virology 158, 1523-1531; Valdes, I., et al. (2009) Vaccine 27, 995-1001). Other immunogen strategies that focus on more complex epitopes or on mimicking the prefusion E dimer are being explored (Manoff, S. B., et al. (2015) Vaccine 33, 7126-7134), but EDIII has the advantage of being relatively small and easy to produce in large quantities. Dengue EDIII has high potential as an immunogen target, but previous attempts to improve its qualities have not been successful. One strategy to decrease the complexity of tetravalent cocktails is to produce EDIII fusion proteins linking EDIIIs from the four serotypes by flexible linkers (“beads on a string”), but this approach resulted in an imbalanced neutralizing titer response in mice and only partial protection in a suckling mice model for DENV1, 2, and 4 (Zhao, H., et al. (2014) PloS one 9, e86573). Recent studies with virus-like particles (VLPs) containing a similar beads-on-a-string design provided protective, but still imbalanced neutralizing antibody titers (Rajpoot, R. K., et al. (2018) Scientific reports 8, 8643; Ramasamy, V. et al. (2018) PLoS neglected tropical diseases 12, e0006191). Nonetheless, the flexible linkers between EDIII domains on such constructs are a liability for proteolysis and may themselves cause immune response. Thus, a preferred approach would be to sculpt single EDIII domains to induce cross-serotype responses. Another strategy is engineering of a “consensus” DIII, in which conserved segments were emphasized (Chen, H. W., et al. (2013) Archives of virology 158, 1523-1531). However, this approach led to DENV2-specific responses in non-human primates NHPs (Chen, H. W. et al. (2013) Arch Virol 158, 1523-1531). A common and unresolved issue for EDIII immunogen development is immunodominance of regions outside of critical neutralizing epitopes, which is addressed herein with structure-guided protein engineering in this work.

The present invention provides a resurfaced Dengue virus glycoprotein subunit E DIII variant (rsD4DIIIs″) comprising variant rsD4DIII-1 (SEQ ID NO:3) or (SEQ ID NO:69), variant rsD4DIII-2 (SEQ ID NO:4) or (SEQ ID NO:71), variant rsD4DIII-3 (SEQ ID NO:5), variant rsD4DIII-4 (SEQ ID NO:6) or (SEQ ID NO:73), variant rsD4DIII-5 (SEQ ID NO:7), variant rsD4DIII-7 (SEQ ID NO:75), variant rsD4DIII-8 (SEQ ID NO:8) or (SEQ ID NO:77), variant rsD4DIII-9 (SEQ ID NO:9) or (SEQ ID NO:79), variant rsD4DIII-10 (SEQ ID NO:10) or (SEQ ID NO:81), variant rsD4DIII-11 (SEQ ID NO:83), variant rsD4DIII-12 (SEQ ID NO:11), variant rsD4DIII-13 (SEQ ID NO:12), variant rsD4DIII-14 (SEQ ID NO:13), or variant rsD4DIII-18 (SEQ ID NO:85).

In some embodiments, the variant consists of the specified variant. In one embodiments, the variant consists essentially of the specified variant, wherein any elements added to the specified variant do not decrease the immunogenic properties of the specified variant.

The resurfaced, engineered Dengue virus glycoprotein subunit E DIII variants have the amino acid sequences, or nucleotide sequences, set forth below. The underlined portions of the sequences below correspond to the amino acid residues set forth for the corresponding sequences in Table 1.

```
Dengue virus 4 strain TVP-376 amino acid sequence GenBank: AGS14893.1
WILD-TYPE
GMSYTMCS GKFSIDKEMA ETQHGTTVVK VKYEGAGAPC KVPIEIRDVN
KEKVVGRIIS STPFAENTNS VTNIELEPPF GDSYIVIGVG DSALTLHWFR
KGSSIGK (SEQ ID NO: 1)
Dengue virus 4 strain TVP-376 DNA sequence GenBank: KC963424.1 WILD-
TYPE
            ggaa tgtcatacac gatgtgttca ggaaagtttt caattgacaa
agagatggca gaaacacagc atgggacaac agtggtgaaa gtcaagtatg
aaggtgctgg agctccgtgt aaagtcccca tagagataag agatgtaaac
aaggaaaaag tggttgggcg tatcatctca tccacccctT TTgctgagaa
taccaacagt gtaaccaaca tagaattaga accccccttt ggggacagct
acatagtgat aggtgttgga GATagcgcat taacactcca ttggttcagg
aaagggagtt ccattggcaa g (SEQ ID NO: 2)
            ggaa tgtcatacac gatgtgttca ggaaagtttt caattgacaa
agagatggca gaaacacagc atgggacaac agtggtgaaa gtcaagtatg
aaggtgctgg agctccgtgt aaagtcccca tagagataag agatgtaaac
aaggaaaaag tggttgggcg tatcatctca tccacccctT TTgctgagaa
taccaacagt gtaaccaaca tagaattaga accccccttt ggggacagct
acatagtgat aggtgttgga GACagcgcat taacactcca ttggttcagg
aaagggagtt ccattggcaa g (SEQ ID NO: 29)
            ggaa tgtcatacac gatgtgttca ggaaagtttt caattgacaa
agagatggca gaaacacagc atgggacaac agtggtgaaa gtcaagtatg
aaggtgctgg agctccgtgt aaagtcccca tagagataag agatgtaaac
aaggaaaaag tggttgggcg tatcatctca tccacccctT TCgctgagaa
taccaacagt gtaaccaaca tagaattaga accccccttt ggggacagct
acatagtgat aggtgttgga GATagcgcat taacactcca ttggttcagg
aaagggagtt ccattggcaa g (SEQ ID NO: 30)
            ggaa tgtcatacac gatgtgttca ggaaagtttt caattgacaa
agagatggca gaaacacagc atgggacaac agtggtgaaa gtcaagtatg
aaggtgctgg agctccgtgt aaagtcccca tagagataag agatgtaaac
aaggaaaaag tggttgggcg tatcatctca tccacccctT TCgctgagaa
taccaacagt gtaaccaaca tagaattaga accccccttt ggggacagct
acatagtgat aggtgttgga GACagcgcat taacactcca ttggttcagg
aaagggagtt ccattggcaa g (SEQ ID NO: 31)
MPMHDYKDDDDKGSGMS [symbol] MCSGKFSIDKEMA [symbol] GTTVVFNKGEGKGAP
CKVPIEI [symbol] D [symbol] VVG [symbol] IISSTPFAENENSVFNIEEEPPFGDSYIVIGVGDSALTLHWFR [symbol]
GSSIGKFCGRPSG (SEQ ID NO: 67)
Aatgcctatgcatgactacaaggatgatgatgacaaaggcagcggcatgagctacaccatg
tgcagcggcaaattcagcatcgacaaggagatggccgaaacccagcatggcaccaccgtgg
tgaaagtgaaatacgaaggcgcaggtgccccgtgcaaagtgccgatcgaaatccgcgatgt
gaacaaggagaaggtggtgggtcgcattatcagcagcaccccgttcgccgaaaacaccaac
agcgtgaccaacatcgaactggaaccgccgtttggcgatagctatatcgtgattggcgtgg
gcgatagcgccctgaccctgcattggttccgcaaaggcagcagcattggtaaaacatgcgg
ccggccctctggtt (SEQ ID NO: 68)
rsD4DIII-1 amino acid
GMSAAMCS GKFSIDKEMA ETEAGTTVVK VKYEGAGAPC KVPIEIRDVN
KEKVVGRIIS STPFAENTNS VTNIELEPPF GDSYIVIGVG DSALTLHWFR
EGSSIGK (SEQ ID NO: 3)
ggcatgagcgccgccatgtgcagcggcaagttcagcatcgacaaggagatggcc
gagaccgaggccggcaccaccgtggtgaaggtgaagtacgagggcgccggcgccccctgca
aggtgcccatcgagatcagggacgtgaacaaggagaaggtggtgggcaggatcatcagcag
cacccccttcgccgagaacaccaacagcgtgaccaacatcgagctggagccccccttcggc
gacagctacatcgtgatcggcgtgggcgacagcgccctgaccctgcactggttcagggagg
gcagcagcatcggcaag (SEQ ID NO: 32)
MPMHDYKDDDDKGSGMS [symbol] MCSGKFSIDKEMA [symbol] GTTVVKVKYEGAGAPCK
VPIEI [symbol] D [symbol] VVG [symbol] IISSTPFAENTNSVTNIELEPPFGDSYIVIGVGDSALTLHWFR [symbol] G
SSIGKTCGRPSG (SEQ ID NO: 69)
aatgcctatgcatgactacaaggatgatgatgacaaaggcagcggcatgagcgc
tgctatgtgcagcggcaaattcagcatcgacaaggagatggccgaaactgaagctggcacc
accgtggtgaaagtgaaatacgaaggcgcaggtgccccgtgcaaagtgccgatcgaaatcc
gcgatgtgaacaaggagaaggtggtgggtcgcattatcagcagcaccccgttcgccgaaaa
caccaacagcgtgaccaacatcgaactggaaccgccgtttggcgatagctatatcgtgatt
ggcgtgggcgatagcgccctgaccctgcattggttccgcgaaggcagcagcattggtaaaa
catgcggccggccctctggtt (SEQ ID NO: 70)
rsD4DIII-2 amino acid
GMSAAMCS GKFSIDKEMA ETEHGTTWK VKYEGAGAPC KVPIEIRDVN
KEKVVGRIIS STPFAENTNS VTNIELEPPF GDSYIVIGVG DSALTLHWFR
KGSSIGK (SEQ ID NO: 4)
ggcatgagcgccgccatgtgcagcggcaagttcagcatcgacaaggagatggcc
gagaccgagcacggcaccaccgtggtgaaggtgaagtacgagggcgccggcgccccctgca
aggtgcccatcgagatcagggacgtgaacaaggagaaggtggtgggcaggatcatcagcag
cacccccttcgccgagaacaccaacagcgtgaccaacatcgagctggagccccccttcggc
gacagctacatcgtgatcggcgtgggcgacagcgccctgaccctgcactggttcaggaagg
gcagcagcatcggcaag (SEQ ID NO: 33)
MPMHDYKDDDDKGSGMS [symbol] MCSGKFSIDKEMA [symbol] GTTVVKVKYEGAGAP
CKVPIEI [symbol] D [symbol] VVG [symbol] IISSTPFAENTNSVTNIELEPPFGDSYIVIGVGDSALTLHWF
R [symbol] GSSIGKTCGRPSG (SEQ ID NO: 71)
aatgcctatgcatgactacaaggatgatgatgacaaaggcagcggcatgagcgc
tgctatgtgcagcggcaaattcagcatcgacaaggagatggccgaaactgaacatggcacc
accgtggtgaaagtgaaatacgaaggcgcaggtgccccgtgcaaagtgccgatcgaaatcc
gcgatgtgaacaaggagaaggtggtgggtcgcattatcagcagcaccccgttcgccgaaaa
caccaacagcgtgaccaacatcgaactggaaccgccgtttggcgatagctatatcgtgatt
ggcgtgggcgatagcgccctgaccctgcattggttccgcaaaggcagcagcattggtaaaa
catgcggccggccctctggtt (SEQ ID NO: 72)
rsD4DIII-3 amino acid
GMSATMCS GKFSIDKEMA ETPDGTTVVK VKYEGAGAPC KVPIEIRDAD
TEKVVGAIIS STPFAENTNS VTNIELEPPF GDSYIVIGVG DSALTLHWFR
EGSSIGK (SEQ ID NO: 5)
ggcatgagcgccaccatgtgcagcggcaagttcagcatcgacaaggagatggcc
gagacccccgacggcaccaccgtggtgaaggtgaagtacgagggcgccggcgccccctgca
aggtgcccatcgagatcagggacgccgacaccgagaaggtggtgggcgccatcatcagcag
cacccccttcgccgagaacaccaacagcgtgaccaacatcgagctggagccccccttcggc
gacagctacatcgtgatcggcgtgggcgacagcgccctgaccctgcactggttcagggagg
gcagcagcatcggcaag (SEQ ID NO: 34)
rsD4DIII-4 amino acid
GMSDAMCS GKFSIDKEMA ATEHGTTVVK VKYEGAGAPC KVPIEIRDAT
TEAVVGGIIS STPFAENTNS VTNIELEPPF GDSYIVIGVG DSALTLHWFR
EGSSIGK (SEQ ID NO: 6)
ggcatgagcgacgccatgtgcagcggcaagttcagcatcgacaaggagatggcc
gccaccgagcacggcaccaccgtggtgaaggtgaagtacgagggcgccggcgccccctgca
aggtgcccatcgagatcagggacgccaccaccgaggccgtggtgggcggcatcatcagcag
cacccccttcgccgagaacaccaacagcgtgaccaacatcgagctggagccccccttcggc
gacagctacatcgtgatcggcgtgggcgacagcgccctgaccctgcactggttcagggagg
gcagcagcatcggcaag (SEQ ID NO: 35)
MPMHDYKDDDDKGSGMS [symbol] MCSGKFSIDKEMA [symbol] GTTVVKVKYEGAGAPCKV
PIEI [symbol] D [symbol] VVG [symbol] IISSTPFAENTNSVTNIELEPPFGDSYIVIGVGDSALTLHWFR [symbol] GS
SIGKTCGRPSG (SEQ ID NO: 73)
aatgcctatgcatgactacaaggatgatgatgacaaaggcagcggcatgagcta
caccatgtgcagcggcaaattcagcatcgacaaggagatggccgcaactgaacatggcacc
accgtggtgaaagtgaaatacgaaggcgcaggtgccccgtgcaaagtgccgatcgaaatcc
gcgatgctaccacaaaagcagtggtgggtggtattatcagcagcaccccgttcgccgaaaa
caccaacagcgtgaccaacatcgaactggaaccgccgtttggcgatagctatatcgtgatt
ggcgtgggcgatagcgccctgaccctgcattggttccgcgaaggcagcagcattggtaaaa
catgcggccggccctctggtt (SEQ ID NO: 74)
rsD4DIII-5 amino acid
GMSDAMCS GKFSIDKEMA ATPHGTTVVK VKYEGAGAPC KVPIEIRDAA
KEKVVGGIIS STPFAENTNS VTNIELEPPF GDSYIVIGVG DSALTLHWFR
EGSSIGK (SEQ ID NO: 7)
ggcatgagcgacgccatgtgcagcggcaagttcagcatcgacaaggagatggcc
gccaccccccacggcaccaccgtggtgaaggtgaagtacgagggcgccggcgccccctgca
aggtgcccatcgagatcagggacgccgccaaggagaaggtggtgggcggcatcatcagcag
cacccccttcgccgagaacaccaacagcgtgaccaacatcgagctggagccccccttcggc
gacagctacatcgtgatcggcgtgggcgacagcgccctgaccctgcactggttcagggagg
gcagcagcatcggcaag (SEQ ID NO: 36)
rsD4DIII-7 amino acid
MPMHDYKDDDDKGSGMS [symbol] MCSGKFSIDKEMA [symbol] GTTVVKVKYEGAGAPCKV
PIEI [symbol] D [symbol] VVG [symbol] IISSTPFAENTNSVTNIELEPPFGDSYIVIGVGDSALTLHWFR [symbol] GS
SIGKTCGRPSG (SEQ ID NO: 75)
Aatgcctatgcatgactacaaggatgatgatgacaaaggcagcggcatgagcga
tgctatgtgcagcggcaaattcagcatcgacaaggagatggccgaaactgcagatggcacc
accgtggtgaaagtgaaatacgaaggcgcaggtgccccgtgcaaagtgccgatcgaaatcc
gtgatgttgacacagcagcagtggtgggtcgtattatcagcagcaccccgttcgccgaaaa
caccaacagcgtgaccaacatcgaactggaaccgccgtttggcgatagctatatcgtgatt
ggcgtgggcgatagcgccctgaccctgcattggttccgcgcaggcagcagcattggtaaaa
catgcggccggccctctggtt (SEQ ID NO: 76)
rsD4DIII-8 amino acid
GMSDAMCS GKFSIDKEMA ETEDGTTVVK VKYEGAGAPC KVPIEIRDVN
KEKVVGRIIS STPFAENTNS VTNIELEPPF GDSYIVIGVG DSALTLHWFR
EGSSIGK (SEQ ID NO: 8)
ggcatgagcgacgccatgtgcagcggcaagttcagcatcgacaaggagatggcc
gagaccgaggacggcaccaccgtggtgaaggtgaagtacgagggcgccggcgccccctgca
aggtgcccatcgagatcagggacgtgaacaaggagaaggtggtgggcaggatcatcagcag
cacccccttcgccgagaacaccaacagcgtgaccaacatcgagctggagccccccttcggc
gacagctacatcgtgatcggcgtgggcgacagcgccctgaccctgcactggttcagggagg
gcagcagcatcggcaag (SEQ ID NO: 37)
MPMHDYKDDDDKGSGMS [symbol] MCSGKFSIDK [symbol] MA [symbol] GTTVVKVKYKRACAPCK
VPIEI [symbol] D [symbol] VVG [symbol] IISSTPFAENTNSVTNIELEPPFGDSYIVIGVGDSALTLHWFR [symbol]
GSSIGKTCGRPSG (SEQ ID NO: 77)
Aatgcctatgcatgactacaaggatgatgatgacaaaggcagcggcatgagcga
tgctatgtgcagcggcaaattcagcatcgacaaggatatggccgaaactgaagatggcacc
accgtggtgaaagtgaaatacaaacgcgcatgtgccccgtgcaaagtgccgatcgaaatcc
gcaatgtgaacaaggataatgtggtgggtcgcattatcaacaccaccccgttcgccgaaaa
caccaacagcgtgaccaacatcgaactggaaccgccgtttggcgatagctatatcgtgatt
ggcgtgggcgatagcgccctgaccctgcattggttccgcaaaggcagcagcattggtaaaa
catgcggccggccctctggtt (SEQ ID NO: 78)
rsD4DIII-9 amino acid
GMSDAMCS GKFSIDKEMA ETPDGTTVVK VKYEGAGAPC KVPIEIRDVN
KEKVVGRIIS STPFAENTNS VTNIELEPPF GDSYIVIGVG DSALTLHWFR
EGSSIGK (SEQ ID NO: 9)
ggcatgagcgacgccatgtgcagcggcaagttcagcatcgacaaggagatggcc
gagacccccgacggcaccaccgtggtgaaggtgaagtacgagggcgccggcgccccctgca
aggtgcccatcgagatcagggacgtgaacaaggagaaggtggtgggcaggatcatcagcag
cacccccttcgccgagaacaccaacagcgtgaccaacatcgagctggagccccccttcggc
gacagctacatcgtgatcggcgtgggcgacagcgccctgaccctgcactggttcagggagg
gcagcagcatcggcaag (SEQ ID NO: 38)
MPMHDYKDDDDKGSGMS [symbol] MCSGKFSIDKEMA [symbol] GTTVVKVKYEGAGAPCK
VPIEI [symbol] D [symbol] VVG [symbol] IISSTPFAENTNSVTNIELEPPFGDSYIVIGVGDSALTLHWFR [symbol]
GSSIGKTCGRPSG (SEQ ID NO: 79)
aatgcctatgcatgactacaaggatgatgatgacaaaggcagcggcatgagcga
tgctatgtgcagcggcaaattcagcatcgacaaggagatggccgaaactccagatggcacc
accgtggtgaaagtgaaatacgaaggcgcaggtgccccgtgcaaagtgccgatcgaaatcc
gcgatgtgaacaaggagaaggtggtgggtcgcattatcagcagcaccccgttcgccgaaaa
caccaacagcgtgaccaacatcgaactggaaccgccgtttggcgatagctatatcgtgatt
ggcgtgggcgatagcgccctgaccctgcattggttccgcgaaggcagcagcattggtaaaa
catgcggccggccctctggtt (SEQ ID NO: 80)
RsD4DIII-10 amino acid
GMSSAMCS GKFSIDKEMA ATEAGTTVVK VKYEGAGAPC KVPIEIRDAD
KAKVVGGIIS STPFAENTNS VTNIELEPPF GDSYIVIGVG DSALTLHWFR
EGSSIGK (SEQ ID NO: 10)
ggcatgagcagcgccatgtgcagcggcaagttcagcatcgacaaggagatggcc
gccaccgaggccggcaccaccgtggtgaaggtgaagtacgagggcgccggcgccccctgca
aggtgcccatcgagatcagggacgccgacaaggccaaggtggtgggcggcatcatcagcag
cacccccttcgccgagaacaccaacagcgtgaccaacatcgagctggagccccccttcggc
gacagctacatcgtgatcggcgtgggcgacagcgccctgaccctgcactggttcagggagg
gcagcagcatcggcaag (SEQ ID NO: 39)
MPMHDYKDDDDKGSGMS [symbol] MC [symbol] GKFST [symbol] KEMA [symbol] GTTVVKVKYEGAGAPCK
VPIEI [symbol] D [symbol] VVG [symbol] IISSTPFAENTNSVTNIELEPPFGDSYIVIGVGDSALTLHWFR [symbol]
GSSIGKTCGRPSG (SEQ ID NO: 81)
aatgcctatgcatgactacaaggatgatgatgacaaaggcagcggcatgagctc
tgctatgtgcaccggcaaattcagcatcaacaaggagatggccgcaactgaagctggcacc
accgtggtgaaagtgaaatacgaaggcgcaggtgccccgtgcaaagtgccgatcgaaatcc
gtgatgctgacaaagcaaaagtggtgggtggtattatcagcagcaccccgttcgccgaaaa
caccaacagcgtgaccaacatcgaactggaaccgccgtttggcgatagctatatcgtgatt
ggcgtgggcgatagcgccctgaccctgcattggttccgcgaaggcagcagcattggtaaaa
catgcggccggccctctggtt (SEQ ID NO: 82)
rsD4DIII-11 amino acid
MPMHDYKDDDDKGSGMS [symbol] MCSGKFSIDKEMA [symbol] GTTVVKVKYEGAGAPCK
VPIEI [symbol] D [symbol] VVG [symbol] IISSTPFAENTNSVTNIELEPPFGDSYIVIGVGDSALTLHWFR [symbol]
GSSIGKTCGRPSG (SEQ ID NO: 83)
aatgcctatgcatgactacaaggatgatgatgacaaaggcagcggcatgagcta
tgctatgtgcagcggcaaattcagcatcgacaaggagatggccgcagctgcagatggcacc
accgtggtgaaagtgaaatacgaaggcgcaggtgccccgtgcaaagtgccgatcgaaatcc
gtgatgctgccaaagcagcagtggtgggtggtattatcagcagcaccccgttcgccgaaaa
caccaacagcgtgaccaacatcgaactggaaccgccgtttggcgatagctatatcgtgatt
ggcgtgggcgatagcgccctgaccctgcattggttccgcgaaggcagcagcattggtaaaa
catgcggccggccctctggtt (SEQ ID NO: 84)
rsD4DIII-12 amino acid
GMSYAMCS GKFSIDKEMA ATHDGTTVVK VKYEGAGAPC KVPIEIRDVN
KEKVVGRIIS STPFAENTNS VTNIELEPPF GDSYIVIGVG DSALTLHWFR
AGSSIGK (SEQ ID NO: 11)
ggcatgagctacgccatgtgcagcggcaagttcagcatcgacaaggagatggcc
gccacccacgacggcaccaccgtggtgaaggtgaagtacgagggcgccggcgccccctgca
aggtgcccatcgagatcagggacgtgaacaaggagaaggtggtgggcaggatcatcagcag
cacccccttcgccgagaacaccaacagcgtgaccaacatcgagctggagccccccttcggc
gacagctacatcgtgatcggcgtgggcgacagcgccctgaccctgcactggttcagggccg
gcagcagcatcggcaag (SEQ ID NO: 40)
rsD4DIII-13 amino acid
GMSYTMCS GKFSIDKEMA ATEHGTTVVK VKYEGAGAPC KVPIEIRDAT
KEKVVGGIIS STPLAENTNS VTNIELEPPF GDSYIVIGVG NSALTLHWFR
EGSSIGK (SEQ ID NO: 12)
ggcatgagctacaccatgtgcagcggcaagttcagcatcgacaaggagatggcc
gccaccgagcacggcaccaccgtggtgaaggtgaagtacgagggcgccggcgccccctgca
aggtgcccatcgagatcagggacgccaccaaggagaaggtggtgggcggcatcatcagcag
cacccccctggccgagaacaccaacagcgtgaccaacatcgagctggagccccccttcggc
gacagctacatcgtgatcggcgtgggcaacagcgccctgaccctgcactggttcagggagg
gcagcagcatcggcaag (SEQ ID NO: 41)
rsD4DIII-14 amino acid
GMSYTMCS GKFSIDKEMA EAEDGTTVVK VKYEGAGAPC KVPIEIRDAD
EETVVGRIIS STPLAENTNS VTNIELEPPF GDSYIVIGVG NSALTLHWFR
EGSSIGK (SEQ ID NO: 13)
ggcatgagctacaccatgtgcagcggcaagttcagcatcgacaaggagatggcc
gaggccgaggacggcaccaccgtggtgaaggtgaagtacgagggcgccggcgccccctgca
aggtgcccatcgagatcagggacgccgacgaggagaccgtggtgggcaggatcatcagcag
cacccccctggccgagaacaccaacagcgtgaccaacatcgagctggagccccccttcggc
gacagctacatcgtgatcggcgtgggcaacagcgccctgaccctgcactggttcagggagg
gcagcagcatcggcaag (SEQ ID NO: 42)
rsD4DIII-18 amino acid
MPMHDYKDDDDKGSGMS [symbol] MCSGKFSIDKEMA [symbol] GTTVVKVKYEGAGAPCK
VPIEI [symbol] D [symbol] VVG [symbol] IISSTPFAENTNSVTNIELEPPFGDSYIVIGVGDSALTLHWFR [symbol]
GSSIGKTCGRPSG (SEQ ID NO: 85)
aatgcctatgcatgactacaaggatgatgatgacaaaggcagcggcatgagcta
caccatgtgcagcggcaaattcagcatcgacaaggagatggccgaaactccacatggcacc
accgtggtgaaagtgaaatacgaaggcgcaggtgccccgtgcaaagtgccgatcgaaatcc
gtgatgctaccaaagcaaaagtggtgggtcctattatcagcagcaccccgttcgccgaaaa
caccaacagcgtgaccaacatcgaactggaaccgccgtttggcgatagctatatcgtgatt
ggcgtgggcgatagcgccctgaccctgcattggttccgcgaaggcagcagcattggtaaaa
catgcggccggccctctggtt (SEQ ID NO: 86)
Dengue virus 4 strain TVP-986 amino acid GenBank: AGS14894.1 wild-type
GMSYTMCS GKFSIDKEMA ETQHGTTVVK VKYEGAGAPC KVPIEIRDVN
KEKVVGRIIS STPFAENTNS VTNIELEPPE GDSYIVIGVG DSALTLHWFR
K̅GSSIGK (SEQ ID NO: 14)
ggcatgagctacaccatgtgcagcggcaagttcagcatcgacaaggagatggcc
gagacccagcacggcaccaccgtggtgaaggtgaagtacgagggcgccggcgccccctgca
aggtgcccatcgagatcagggacgtgaacaaggagaaggtggtgggcaggatcatcagcag
cacccccttcgccgagaacaccaacagcgtgaccaacatcgagctggagccccccttcggc
gacagctacatcgtgatcggcgtgggcgacagcgccctgaccctgcactggttcaggaagg
gcagcagcatcggcaag (SEQ ID NO: 43)
Dengue virus 4 strain TVP-986 nucleotide sequence GenBank: KC963425.1
wild-type
            ggaa tgtcatacac gatgtgttca ggaaagtttt caattgacaa
agagatggca gaaacacagc atgggacaac agtggtgaaa gtcaagtatg
aaggtgctgg agctccgtgt aaagtcccca tagagataag agatgtaaac
aaggaaaaag tggttgggcg tatcatctca tccacccctT TGgctgagaa
taccaacagt gtaaccaaca tagaattaga accccccttt ggggacagct
acatagtgat aggtgttgga AACagcgcat taacactcca ttggttcagg
aaagggagtt ccattggcaa g (SEQ ID NO: 15)
```

Also provided are resurfaced DENV-4 EDIII variants encoded by a nucleic acid molecule having at least 70%, 75%, 80%, 85%, 90%, 95%, 96%, 97%, 98%, or 99% sequence identity to the nucleotide sequence set forth in SEQ ID NO:2, 29, 30, 31, 32, 33, 34, 35, 36, 37, 38, 39, 40, 41, 42, 43, 70, 72, 74, 76, 78, 80, 82, 84, or 86. Also provided are resurfaced DENV-4 EDIII variants encoded by a nucleic acid molecule having at least 70%, 75%, 80%, 85%, 90%, 95%, 96%, 97%, 98%, or 99% sequence identity to the nucleotide sequence set forth in or SEQ ID NO:15, 43, or 68.

In some embodiments, the vaccine composition comprises at least one of the specified variant. In some embodiments, the vaccine composition comprises at least two, three, four, five, six, seven, eight, nine, ten, or more of the specified variant. In some embodiments, the vaccine composition comprises at lease one variant selected from the group consisting of variant rsD4DIII-1 (SEQ ID NO:3) or (SEQ ID NO:69), variant rsD4DIII-2 (SEQ ID NO:4) or (SEQ ID NO:71), variant rsD4DIII-3 (SEQ ID NO:5), variant rsD4DIII-4 (SEQ ID NO:6) or (SEQ ID NO:73), variant rsD4DIII-5 (SEQ ID NO:7), variant rsD4DIII-7 (SEQ ID NO:75), variant rsD4DIII-8 (SEQ ID NO:8) or (SEQ ID NO:77), variant rsD4DIII-9 (SEQ ID NO:9) or (SEQ ID NO:79), variant rsD4DIII-10 (SEQ ID NO:10) or (SEQ ID NO:81), variant rsD4DIII-11 (SEQ ID NO:83), variant rsD4DIII-12 (SEQ ID NO:11), variant rsD4DIII-13 (SEQ ID NO:12), variant rsD4DIII-14 (SEQ ID NO:13), and variant rsD4DIII-18 (SEQ ID NO:85), or combinations thereof. In some embodiments, a mulitivalent vaccine comprises at least two or more variants selected from the group consisting of variant rsD4DIII-1 (SEQ ID NO:3) or (SEQ ID NO:69), variant rsD4DIII-2 (SEQ ID NO:4) or (SEQ ID NO:71), variant rsD4DIII-3 (SEQ ID NO:5), variant rsD4DIII-4 (SEQ ID NO:6) or (SEQ ID NO:73), variant rsD4DIII-5 (SEQ ID NO:7), variant rsD4DIII-7 (SEQ ID NO:75), variant rsD4DIII-8 (SEQ ID NO:8) or (SEQ ID NO:77), variant rsD4DIII-9 (SEQ ID NO:9) or (SEQ ID NO:79), variant rsD4DIII-10 (SEQ ID NO:10) or (SEQ ID NO:81), variant rsD4DIII-11 (SEQ ID NO:83), variant rsD4DIII-12 (SEQ ID NO:11), variant rsD4DIII-13 (SEQ ID NO:12), variant rsD4DIII-14 (SEQ ID NO:13), and variant rsD4DIII-18 (SEQ ID NO:85), or combinations thereof. When used in the vaccination studies, in some embodiments, the rsD4DIIIs variants were engineered with a C-term SpyTag sequence to facilitate aaLS nanoparticle conjugation. In some embodiments, the SpyTag Amino acid Sequence is GSGSMAHIVMVDAYKPTK (SEQ ID NO: 65). In some embodiments, the SpyTag Nucleotide Sequence is

(SEQ ID NO: 66)
GGTTCTGGTTCTATGGCTCACATCGTTATGGTTGACGCTTACAAACCGACC
AAA.

Zika Virus

The present invention provides a resurfaced ZIKV EDIII variant comprising variant rsZDIII-1.20 (SEQ ID NO:18) or (SEQ ID NO:57), variant rsZDIII-1.39 (SEQ ID NO:19) or (SEQ ID NO:59), variant rsZDIII-1.48 (SEQ ID NO:20), variant rsZDIII-1.69 (SEQ ID NO:21), variant rsZDIII-1.74 (SEQ ID NO:22), variant rsZDIII-2.16 (SEQ ID NO:23) or (SEQ ID NO:61), variant rsZDIII-2.39 (SEQ ID NO:24) or (SEQ ID NO:63), variant rsZDIII-2.50 (SEQ ID NO:25), variant rsZDIII-1.8 (SEQ ID NO:26), variant rsZDIII-1.25 (SEQ ID NO:27), or variant rsZDIII-1.27 (SEQ ID NO:28).

In one embodiment, the variant consists of the specified variant. In one embodiment, the variant consists essentially of the specified variant, wherein any elements added to the specified variant do not decrease the immunogenic properties of the specified variant.

The resurfaced, engineered Ziki virus EDIII variants have the amino acid sequences, or nucleotide sequences, set forth below. The underlined portions of the sequences below correspond to the amino acid residues set forth for the corresponding sequences in Table 2.

Ziki virus wild-type amino acid sequence GenBank: AMO03410.2
GVSYSLCTA AFTFTKIPAE TLHGTVTVEV QYAGTDGPCK VPAQMAVDMQ
TLTPVGRLIT ANPVITESTE NSKMMLELDP PFGDSYIVIG VGEKKITHHW
HRSGSTIGK (SEQ ID NO: 16)
Ziki virus wild-type nucleotide sequence GenBank: KU866423.2
ggtgtgagctatagcctgtgcaccgccgccttcaccttcaccaaaattccggcc
gaaaccctgcatggcaccgtgaccgtggaagtgcagtatgccggtaccgatggtccgtgca
aagttccggcccagatggccgtggatatgcagaccctgaccccggtgggtcgcctgattac
cgccaatccggtgattaccgagagcaccgagaacagcaagatgatgctggaactggatccg
ccgttcggtgatagctatatcgtgattggcgtgggcgagaagaagatcacccatcattggc
atcgcagcggtagcaccatcggcaaa (SEQ ID NO: 17)
GVSYSLCTAAFTFTKIPAETLHGTVTVEVQYAGTDGPCKVPAQMAVDMQTLTPV
GRLITANPVITESTENSKMMLELDPPFGDSYIVIGVGEKKITHHWHRSGSTIGK (SEQ
ID NO: 55)
GGTGTGAGCTATAGCCTGTGCACCGCCGCCTTCACCTTCACCAAAATTCCGGCC
GAAACCCTGGATGGCACCGTGACCGTGGAAGTGCAGTATGCCGGTACCGATGGTCCGTGCA
AAGTTCCGGCCCAGATGGCCGTGGATATGCAGACCCTGACCCCGGTGGGTCGCCTGATTAC
CGCCAATCCGGTGATTACCGAGAGCACCGAGAACAGCAAGATGATGCTGGAACTGGATCCG
CCGTTCGGTGATAGCTATATCGTGATTGGCGTGGGCGAGAAGAAGATCACCCATCATTGGC
ATCGCAGCGGTAGCACCATCGGCAAA (SEQ ID NO: 56)
rsZDIII-1.20
GVSYSLCTA AFTFAKTPAE TLHGTVTVEV QYAGTDGPCK VPAQMAVDMA
AATPVGRLIT ANPVITESTE NSKMVLELDP PFGDSYIVIG VGEKKITHHW
HRSGSTIGK (SEQ ID NO: 18)
ggcgtgagctacagcctgtgcaccgccgccttcaccttcgccaagacccccgcc
gagaccctgcacggcaccgtgaccgtggaggtgcagtacgccggcaccgacggcccctgca
aggtgcccgcccagatggccgtggacatggccgccgccacccccgtgggcaggctgatcac
cgccaaccccgtgatcaccgagagcaccgagaacagcaagatggtgctggagctggacccc
cccttcggcgacagctacatcgtgatcggcgtgggcgagaagaagatcacccaccactggc
acaggagcggcagcaccatcggcaag (SEQ ID NO: 44)
GVSYSLCTAAFTFAKTPAETLHGTVTVEVQYAGTDGPCKVPAQMAVDMAALAPV
GRLITANPVITESTENSKMVLELDPPFGDSYIVIGVGEKKITHHWHRSGSTIGK (SEQ
ID NO: 57)
GGTGTGAGCTATAGCCTGTGCACCGCCGCCTTCACCTTCGCTAAAACTCCGGCC
GAAACCCTGCATGGCACCGTGACTGTGGAAGTGCAGTATGCCGGTACCGATGGTCCGTGCA
AAGTTCCGGCCCAGATGGCCGTGGATATGGCAGCTCTGGCTCCGGTGGGTCGCCTGATTAC
CGCCAATCCGGTGATTACCGAGAGCACCGAGAACAGCAAAATGGTGCTGGAACTGGATCCG
CCGTTCGGTGATAGCTATATCGTGATTGGCGTGGGCGAGAAGAAGATCACCCATCATTGGC
ATCGCAGCGGTAGCACCATCGGCAAA (SEQ ID NO: 58)
rsZDIII-1.39
GVSYSLCTA AFTFTAIPAE TLHGTVTVEV QYAGTDGPCK VPAQMAVDMQ
AATPVGRLIT ANPVITESTE NSKMVLELDP PFGDSYIVIG VGEKKITHHW
HRSGSTIGK (SEQ ID NO: 19)
ggcgtgagctacagcctgtgcaccgccgccttcaccttcaccgccatccccgcc
gagaccctgcacggcaccgtgaccgtggaggtgcagtacgccggcaccgacggcccctgca
aggtgcccgcccagatggccgtggacatgcaggccgccacccccgtgggcaggctgatcac
cgccaaccccgtgatcaccgagagcaccgagaacagcaagatggtgctggagctggacccc
cccttcggcgacagctacatcgtgatcggcgtgggcgagaagaagatcacccaccactggc
acaggagcggcagcaccatcggcaag (SEQ ID NO: 45)
GVSYSLCTAAFTFTAIPAETLHGTVTVEVQYAGTDGPCKVPAQMAVDMQALAPVGRLITAN
PVITESTENSKMVLELDPPFGDSYIVIGVGEKKITHHWHRSGSTIGK (SEQ ID NO:
59)
GGTGTGAGCTATAGCCTGTGCACCGCCGCCTTCACCTTCACTGCAATTCCGGCC
GAAACCCTGCATGGCACCGTGACTGTGGAAGTGCAGTATGCCGGTACCGATGGTCCGTGCA
AAGTTCCGGCCCAGATGGCCGTGGATATGCAAGCTCTGGCTCCGGTGGGTCGCCTGATTAC
CGCC7XATCCGGTGATTACCGAGAGCACCGAGAACAGCAAAATGGTGCTGGAACTGGATCCG
CCGTTCGGTGATAGCTATATCGTGATTGGCGTGGGCGAGIVkGAAGATCACCCATCATTGGC
ATCGCAGCGGTAGCACCATCGGCAAA (SEQ ID NO: 60)
rsZDIII-1.48
GVSYSLCTA AFTFAKAPAE TLHGTVAVEV QYAGTDGPCK VPAQMAVDMA
AATPVGRLIT ANPVITESTE NSKMTLELDP PFGDSYIVIG VGEKKITHHW
HRSGSTIGK (SEQ ID NO: 20)
ggcgtgagctacagcctgtgcaccgccgccttcaccttcgccaaggcccccgcc
gagaccctgcacggcaccgtggccgtggaggtgcagtacgccggcaccgacggcccctgca
aggtgcccgcccagatggccgtggacatggccgccgccacccccgtgggcaggctgatcac
cgccaaccccgtgatcaccgagagcaccgagaacagcaagatgaccctggagctggacccc
cccttcggcgacagctacatcgtgatcggcgtgggcgagaagaagatcacccaccactggc
acaggagcggcagcaccatcggcaag (SEQ ID NO: 46)
rsZDIII-1.69
GVSYSLCTA AFTFATAPAE TLHGTVTVEV QYAGTDGPCK VPAQMAVAMQ
ATTPVGRLIT ANPVITESTE NSKMTLELDP PFGDSYIVIG VGEKKITHHW
HRSGSTIGK (SEQ ID NO: 21)
ggcgtgagctacagcctgtgcaccgccgccttcaccttcgccaccgcccccgcc
gagaccctgcacggcaccgtgaccgtggaggtgcagtacgccggcaccgacggcccctgca
aggtgcccgcccagatggccgtggccatgcaggccaccacccccgtgggcaggctgatcac
cgccaaccccgtgatcaccgagagcaccgagaacagcaagatgaccctggagctggacccc
cccttcggcgacagctacatcgtgatcggcgtgggcgagaagaagatcacccaccactggc
acaggagcggcagcaccatcggcaag (SEQ ID NO: 47)
rsZDIII-1.74
GVSYSLCTA AFTFAKAPAE TLHGTVAVEV QYAGTDGPCK VPAQMAVDMQ
AATPVGRLIT ANPVITESTE NSAMVLELDP PFGDSYIVIG VGEKKITHHW
HRSGSTIGK (SEQ ID NO: 22)
ggcgtgagctacagcctgtgcaccgccgccttcaccttcgccaaggcccccgcc
gagaccctgcacggcaccgtggccgtggaggtgcagtacgccggcaccgacggcccctgca
aggtgcccgcccagatggccgtggacatgcaggccgccacccccgtgggcaggctgatcac
cgccaaccccgtgatcaccgagagcaccgagaacagcgccatggtgctggagctggacccc
cccttcggcgacagctacatcgtgatcggcgtgggcgagaagaagatcacccaccactggc
acaggagcggcagcaccatcggcaag (SEQ ID NO: 48)
rsZDIII-2.16
GVSYSLCTA AFTFTVEPAE TLHGTVTVEV QYAGTDGPCK VPAQMAVDME
TATPVGRLIT ANPVITESTE NSKMMLELDP PFGDSYIVIG VGEKKITHHW
HRSGSTIGK (SEQ ID NO: 23)
ggcgtgagctacagcctgtgcaccgccgccttcaccttcaccgtggagcccgcc
gagaccctgcacggcaccgtgaccgtggaggtgcagtacgccggcaccgacggcccctgca
aggtgcccgcccagatggccgtggacatggagaccgccacccccgtgggcaggctgatcac
cgccaaccccgtgatcaccgagagcaccgagaacagcaagatgatgctggagctggacccc
cccttcggcgacagctacatcgtgatcggcgtgggcgagaagaagatcacccaccactggc
acaggagcggcagcaccatcggcaag (SEQ ID NO: 49)
GVSYSLCTAAFTFTEVPAETLHGTVTVEVQYAGTDGPCKVPAQMAVDMETLAPV
GRLITANPVITESTENSKMMLELDPPFGDSYIVIGVGEKKITHHWHRSGSTIGK (SEQ
ID NO: 61)
GGTGTGAGCTATAGCCTGTGCACCGCCGCCTTCACCTTCACTGAAGTTCCGGCC
GAAACCCTGCATGGCACCGTGACTGTGGAAGTGCAGTATGCCGGTACCGATGGTCCGTGCA
AAGTTCCGGCCCAGATGGCCGTGGATATGGAAACTCTGGCTCCGGTGGGTCGCCTGATTAC
CGCCAATCCGGTGATTACCGAGAGCACCGAGAACAGCAAAATGATGCTGGAACTGGATCCG
CCGTTCGGTGATAGCTATATCGTGATTGGCGTGGGCGAGAAGAAGATCACCCATCATTGGC
ATCGCAGCGGTAGCACCATCGGCAAA (SEQ ID NO: 62)
rsZDIII-2.39
GVSYSLCTA AFTFTEAPAE TLHGTVTVAV QYAGTDGPCK VPAQMAVDME
TATPVGRLIT ANPVITESTE NSKMVLELDP PFGDSYIVIG VGEKKITHHW
HRSGSTIGK (SEQ ID NO: 24)
ggcgtgagctacagcctgtgcaccgccgccttcaccttcaccgaggcccccgcc
gagaccctgcacggcaccgtgaccgtggccgtgcagtacgccggcaccgacggcccctgca
aggtgcccgcccagatggccgtggacatggagaccgccacccccgtgggcaggctgatcac
cgccaaccccgtgatcaccgagagcaccgagaacagcaagatggtgctggagctggacccc
cccttcggcgacagctacatcgtgatcggcgtgggcgagaagaagatcacccaccactggc
acaggagcggcagcaccatcggcaag (SEQ ID NO: 50)
GVSYSLCTTVAFTFTEAPAETLHGTVTVAVQYAGTDGPCKVPAQMAVDMETLAPV
GRLITANPVITESTENSKMVLELDPPFGDSYIVIGVGEKKITHHWHRSGSTIGK (SEQ
ID NO: 63)
GGTGTGAGCTATAGCCTGTGCACCGCCGCCTTCACCTTCACTGAAGCTCCGGCC
GAAACCCTGCATGGCACCGTGACTGTGGCAGTGCAGTATGCCGGTACCGATGGTCCGTGCA
AAGTTCCGGCCCAGATGGCCGTGGATATGGAAACTCTGGCTCCGGTGGGTCGCCTGATTAC
CGCCAATCCGGTGATTACCGAGAGCACCGAGAACAGCAAAATGGTGCTGGAACTGGATCCG
CCGTTCGGTGATAGCTATATCGTGATTGGCGTGGGCGAGAAGAAGATCACCCATCATTGGC
ATCGCAGCGGTAGCACCATCGGCAAA (SEQ ID NO: 64)
rsZDIII-2.50
GVSYSLCTA AFT FATIPAE TLHGTVAVEV QYAGTDGPCK VPAQMAVEME
TATPVGRLIT ANPVITESTE NSKMMLELDP PFGDSYIVIG VGEKKITHHW
HRSGSTIGK (SEQ ID NO: 25)
ggcgtgagctacagcctgtgcaccgccgccttcaccttcgccaccatccccgcc
gagaccctgcacggcaccgtggccgtggaggtgcagtacgccggcaccgacggcccctgca
aggtgcccgcccagatggccgtggagatggagaccgccacccccgtgggcaggctgatcac
cgccaaccccgtgatcaccgagagcaccgagaacagcaagatgatgctggagctggacccc
cccttcggcgacagctacatcgtgatcggcgtgggcgagaagaagatcacccaccactggc
acaggagcggcagcaccatcggcaag (SEQ ID NO: 51)
rsZDIII-1.8
GVSYSLCTA AFTFAAIPAE TLHGTVAVAV QYAGTDGPCK VPAQMAVDMP
TATPVGRLIT ANPVITESTE NSKMTLELDP PFGDSYIVIG VGEKKITHHW
HRSGSTIGK (SEQ ID NO: 26)
ggcgtgagctacagcctgtgcaccgccgccttcaccttcgccgccatccccgcc
gagaccctgcacggcaccgtggccgtggccgtgcagtacgccggcaccgacggcccctgca
aggtgcccgcccagatggccgtggacatgcccaccgccacccccgtgggcaggctgatcac
cgccaaccccgtgatcaccgagagcaccgagaacagcaagatgaccctggagctggacccc
cccttcggcgacagctacatcgtgatcggcgtgggcgagaagaagatcacccaccactggc
acaggagcggcagcaccatcggcaag (SEQ ID NO: 52)
rsZDIII-1.25
GVSYSLCTA AFTFAAAPAE TLHGTVAVEV QYAGTDGPCK VPAQMAVDME
AATPVGRLIT ANPVITESTE NSKMALELDP PFGDSYIVIG VGEKKITHHW
HRSGSTIGK (SEQ ID NO: 27)
ggcgtgagctacagcctgtgcaccgccgccttcaccttcgccgccgcccccgcc
gagaccctgcacggcaccgtggccgtggaggtgcagtacgccggcaccgacggcccctgca
aggtgcccgcccagatggccgtggacatggaggccgccacccccgtgggcaggctgatcac
cgccaaccccgtgatcaccgagagcaccgagaacagcaagatggccctggagctggacccc
cccttcggcgacagctacatcgtgatcggcgtgggcgagaagaagatcacccaccactggc
acaggagcggcagcaccatcggcaag (SEQ ID NO: 53)
rsZDIII-1.28
GVSYSLCTA AFTFTKIPAE TLHGTVTVEV QYAGTDGPCK VPAQMAVDMQ
TLTPVGRLIT ANPVITESTE NSKMMLELDP PFGDSYIVIG VGEKKITHHW
HRSGSTIGK (SEQ ID NO: 28)
ggcgtgagctacagcctgtgcaccgccgccttcaccttcaccaagatccccgcc
gagaccctgcacggcaccgtgaccgtggaggtgcagtacgccggcaccgacggcccctgca
aggtgcccgcccagatggccgtggacatgcagaccctgacccccgtgggcaggctgatcac
cgccaaccccgtgatcaccgagagcaccgagaacagcaagatgatgctggagctggacccc
cccttcggcgacagctacatcgtgatcggcgtgggcgagaagaagatcacccaccactggc
acaggagcggcagcaccatcggcaag (SEQ ID NO: 54)

Also provided are resurfaced ZIKV EDIII variants encoded by a nucleic acid molecule having at least 70%, 75%, 80%, 85%, 90%, 95%, 96%, 97%, 98%, or 99% sequence identity to the nucleotide sequence set forth in SEQ ID NO:17, 44, 45, 46, 47, 48, 49, 50, 51, 52, 53, 54, 56, 58, 60, 62, or 64.

The compositions and methods of the present invention also encompass modifications of the rsD4DIIIs variants and rsZDIIIs described herein. A modification of a variant, is a polypeptide that differs from the recited polypeptide only in conservative substitutions and/or alterations, such that the therapeutic, antigenic and/or immunogenic properties of the variants are retained. Polypeptide modifications preferably exhibit at least about 70%, more preferably at least about 90% and most preferably at least about 95% homology to the identified variants. For polypeptides with immunoreactive properties, variants can, alternatively, be identified by modifying the amino acid sequence of one of the above polypeptides, and evaluating the immunoreactivity of the modified polypeptide. Such modified sequences can be prepared and tested using, for example, the representative procedures described herein.

As used herein, a “conservative substitution” is one in which an amino acid is substituted for another amino acid that has similar properties, such that one skilled in the art of peptide chemistry would expect the secondary structure and hydropathic nature of the polypeptide to be substantially unchanged.

The rsD4DIIIs variants and rsZDIIIs described herein can also, or alternatively, contain other modifications, including the deletion or addition of amino acids that have minimal influence on the antigenic properties, secondary structure and hydropathic nature of the polypeptide. For example, a polypeptide can be conjugated to a signal (or leader) sequence at the N-terminal end of the protein which co-translationally or post-translationally directs transfer of the protein. The polypeptide can also be conjugated to a linker or other sequence for ease of synthesis, purification or identification of the polypeptide (e.g., poly-His), or to enhance binding of the polypeptide to a solid support.

The present invention also encompasses proteins and polypeptides, variants thereof, or those having amino acid sequences analogous to the amino acid sequences of antigenic polypeptides described herein. Such polypeptides are defined herein as antigenic analogs (e.g., homologues), or mutants or derivatives. “Analogous” or “homologous” amino acid sequences refer to amino acid sequences with sufficient identity of any one of the amino acid sequences so as to possess the biological activity (e.g., the ability to elicit a protective immune response to Dengue or Zika virus) of any one of the native polypeptides. For example, an analog polypeptide can be produced with “silent” changes in the amino acid sequence wherein one, or more, amino acid residues differ from the amino acid residues of any one of the protein, yet still possesses the function or biological activity of the antigen peptide. Examples of such differences include additions, deletions or substitutions of residues of the amino acid sequence of antigen peptides. Also encompassed by the present invention are analogous polypeptides that exhibit greater, or lesser, biological activity of any one of the proteins of the present invention. Such polypeptides can be expressed by mutating (e.g., substituting, deleting or adding) nucleic acid residues of any of the sequences described herein. Such mutations can be performed using methods described herein and those known in the art. In particular, the present invention relates to homologous polypeptide molecules having at least about 70% (e.g., 75%, 80%, 85%, 90% or 95%) identity or similarity with SEQ ID NO: 1-28, or combination thereof. Percent “identity” refers to the amount of identical nucleotides or amino acids between two nucleotides or amino acid sequences, respectfully. As used herein, “percent similarity” refers to the amount of similar or conservative amino acids between two amino acid sequences.

Homologous polypeptides can be determined using methods known to those of skill in the art. Initial homology searches can be performed at NCBI against the GenBank, EMBL and SwissProt databases using, for example, the BLAST network service. Altschuler, S. F., et al., J. Mol. Biol., 215:403 (1990), Altschuler, S. F., Nucleic Acids Res., 25:3389-3402 (1998). Computer analysis of nucleotide sequences can be performed using the MOTIFS and the FindPatterns subroutines of the Genetics Computing Group (GCG, version 8.0) software. Protein and/or nucleotide comparisons were performed according to Higgins and Sharp (Higgins, D. G. and Sharp, P. M., Gene, 73:237-244 (1988) e.g., using default parameters).

Additionally, the individual isolated rsD4DIIIs variants and rsZDIIIs variants of the present invention are biologically active or functional and can elicit a broadly neutralizing antibody response against all four serotypes of Dengue virus and/or Zika virus. The present invention includes fragments of these isolated amino acid sequences that still possess the function or biological activity of the sequence. Fragments, homologues, or analogous polypeptides can be evaluated for biological activity, as described herein.

In some embodiments, the vaccine composition comprises at least one of the specified variant. In some embodiments, the vaccine composition comprises at least two, three, four, five, six, seven, eight, nine, ten, or more of the specified variant. In some embodiments, the vaccine composition comprises at lease one variant selected from the group consisting of variant rsZDIII-1.20 (SEQ ID NO:18) or (SEQ ID NO:57), variant rsZDIII-1.39 (SEQ ID NO:19) or (SEQ ID NO:59), variant rsZDIII-1.48 (SEQ ID NO:20), variant rsZDIII-1.69 (SEQ ID NO:21), variant rsZDIII-1.74 (SEQ ID NO:22), variant rsZDIII-2.16 (SEQ ID NO:23) or (SEQ ID NO:61), variant rsZDIII-2.39 (SEQ ID NO:24) or (SEQ ID NO:63), variant rsZDIII-2.50 (SEQ ID NO:25), variant rsZDIII-1.8 (SEQ ID NO:26), variant rsZDIII-1.25 (SEQ ID NO:27), and variant rsZDIII-1.27 (SEQ ID NO:28), or combinations thereof. In some embodiments, a mulitivalent vaccine comprises at least two or more variants selected from the group consisting of variant rsZDIII-1.20 (SEQ ID NO:18) or (SEQ ID NO:57), variant rsZDIII-1.39 (SEQ ID NO:19) or (SEQ ID NO:59), variant rsZDIII-1.48 (SEQ ID NO:20), variant rsZDIII-1.69 (SEQ ID NO:21), variant rsZDIII-1.74 (SEQ ID NO:22), variant rsZDIII-2.16 (SEQ ID NO:23) or (SEQ ID NO:61), variant rsZDIII-2.39 (SEQ ID NO:24) or (SEQ ID NO:63), variant rsZDIII-2.50 (SEQ ID NO:25), variant rsZDIII-1.8 (SEQ ID NO:26), variant rsZDIII-1.25 (SEQ ID NO:27), and variant rsZDIII-1.27 (SEQ ID NO:28). When used in the vaccination studies, in some embodiments, the rsZDIIIs variants were engineered with a C-term SpyTag sequence to facilitate aaLS nanoparticle conjugation. In some embodiments, the SpyTag Amino acid Sequence is GSGSMAHIVMVDAYKPTK (SEQ ID NO: 65). In some embodiments, the SpyTag Nucleotide Sequence is GGTTCTGGTTCTATGGCTCACATCGTTATGGTTGACGCTTACAAACCGACCAAA (SEQ ID NO: 66).

In some embodiments, a multivalent vaccine comprises a combination of at least one rsD4DIII and at least one rsZDIII. In some a multivalent vaccine comprises (a) at least one rsD4DIII selected from the group consisting of variant rsD4DIII-1 (SEQ ID NO:3) or (SEQ ID NO:69), variant rsD4DIII-2 (SEQ ID NO:4) or (SEQ ID NO:71), variant rsD4DIII-3 (SEQ ID NO:5), variant rsD4DIII-4 (SEQ ID NO:6) or (SEQ ID NO:73), variant rsD4DIII-5 (SEQ ID NO:7), variant rsD4DIII-7 (SEQ ID NO:75), variant rsD4DIII-8 (SEQ ID NO:8) or (SEQ ID NO:77), variant rsD4DIII-9 (SEQ ID NO:9) or (SEQ ID NO:79), variant rsD4DIII-10 (SEQ ID NO:10) or (SEQ ID NO:81), variant rsD4DIII-11 (SEQ ID NO:83), variant rsD4DIII-12 (SEQ ID NO:11), variant rsD4DIII-13 (SEQ ID NO:12), variant rsD4DIII-14 (SEQ ID NO:13), and variant rsD4DIII-18 (SEQ ID NO:85), combined with (b) at least one rsZDIII selected from the group consisting of variant rsZDIII-1.20 (SEQ ID NO:18) or (SEQ ID NO:57), variant rsZDIII-1.39 (SEQ ID NO:19) or (SEQ ID NO:59), variant rsZDIII-1.48 (SEQ ID NO:20), variant rsZDIII-1.69 (SEQ ID NO:21), variant rsZDIII-1.74 (SEQ ID NO:22), variant rsZDIII-2.16 (SEQ ID NO:23) or (SEQ ID NO:61), variant rsZDIII-2.39 (SEQ ID NO:24) or (SEQ ID NO:63), variant rsZDIII-2.50 (SEQ ID NO:25), variant rsZDIII-1.8 (SEQ ID NO:26), variant rsZDIII-1.25 (SEQ ID NO:27), and variant rsZDIII-1.27 (SEQ ID NO:28).

Further provided herein are dimers and oligomers comprising any of the rsD4DIII variants or rsZDIII variants disclosed herein. The dimer or oligomer can contain a C-terminal disulfide-bonded leucine zipper dimerization domain (Stewart, A., et al. (2012) J of Immunol Methods 376, 150-155). Stimulation of B-cell receptors (BCRs) for affinity maturation requires cross-linking of BCRs and thus, dimers or higher order oligomers may be beneficial. In addition, the serum stability of dimers and higher order oligomers may be better than monomers because of the increased size, which minimizes renal clearance, and potential resistance to degradation.

Also provided is a virion of an isolated, recombinant Dengue 4 virus comprising any of the rsD4DIIIs variants or dimers or oligomers disclosed herein. Also provided is a virion of an isolated, recombinant Zika virus comprising any of the rsZDIIIs variants or dimers or oligomers disclosed herein.

Also provided is a Dengue virus vaccine composition comprising any of the rsD4DIIIs variants, or dimers or oligomers, or virions disclosed herein. Also provided is a Zika virus vaccine composition comprising any of the rsZDIIIs variants, or dimers or oligomers, or virions disclosed herein. The vaccine composition can further comprise an immunological adjuvant.

Also provided is a method of eliciting an immune response in a subject comprising administering to the subject any of the rsD4DIIIs and rsZDIIIs variants, or dimers or oligomers, or virions, or vaccines disclosed herein in an amount effective to elicit an immune response in a subject.

Also provided is a method of vaccinating a subject for Dengue virus infection comprising administering to the subject any of the rsD4DIIIs variants, or dimers or oligomers, or virions, or vaccines disclosed herein in an amount effective to vaccinate a subject for Dengue virus.

Also provided is a method of immunizing a subject against Dengue virus infection comprising administering to the subject any of the rsD4DIIIs variants, or dimers or oligomers, or virions, or vaccines disclosed herein in an amount effective to immunize a subject against Dengue virus.

Also provided is a method of vaccinating a subject for Zika virus infection comprising administering to the subject any of the rsZDIIIs variants, or dimers or oligomers, or virions, or vaccines disclosed herein in an amount effective to vaccinate a subject for Dengue virus.

Also provided is a method of immunizing a subject against Zika virus infection comprising administering to the subject any of the rsZDIIIs variants, or dimers or oligomers, or virions, or vaccines disclosed herein in an amount effective to immunize a subject against Dengue virus.

Also provided is a method of treating a Dengue virus infection in a subject or treating a disease caused by a Dengue virus infection in a subject comprising administering to the subject any of the rsD4DIIIs variants, or dimers or oligomers, or virions, or vaccines disclosed herein in an amount effective to treat a Dengue virus infection or treat a disease caused by a Dengue virus infection in a subject. The subject being treated can have, for example, one or more of Dengue Hemorrhagic Fever (DHF) and Dengue Shock Syndrome (DSS).

Preferably, the variants, dimers, oligomers, virions and vaccines disclosed herein are effective against all Dengue virus serotypes.

Also provided is a method of treating a Zika virus infection in a subject or treating a disease caused by a Zika virus infection in a subject comprising administering to the subject any of the rsZDIIIs variants, or dimers or oligomers, or virions, or vaccines disclosed herein in an amount effective to treat a Zika virus infection or treat a disease caused by a Zika virus infection in a subject.

Preferably, the variants, dimers, oligomers, virions and vaccines disclosed herein are effective against all Zika virus serotypes.

The subject can be any animal, and is preferably a human subject.

Formulations and Pharmaceutical Compositions

Formulations and pharmaceutical compositions comprising any of the rsD4DIIIs and rsZDIIIs variants described herein may be formulated according to general pharmaceutical practice (see, for example, “Remington's Pharmaceutial Sciences” and “Encyclopedia of Pharmaceutical Technology”, J. Swarbrick, and J. C. Boylan (Eds.), Marcel Dekker, Inc: New York, 1988).

Pharmaceutically acceptable carriers, vehicles, and/or excipients for the formulations and pharmaceutical compositions comprising any of the rsD4DIIIs and rsZDIIIs variants described herein can be routinely selected for a particular use by those skilled in the art. These include, but are not limited to, solvents, buffering agents, inert diluents or fillers, suspending agents, dispersing or wetting agents, preservatives, stabilizers, chelating agents, emulsifying agents, anti-foaming agents, gel-forming agents, ointment bases, penetration enhancers, humectants, and emollients.

Examples of solvents are water, alcohols, vegetable, marine and mineral oils, polyethylene glycols, propylene glycols, glycerol, and liquid polyalkylsiloxanes. Inert diluents or fillers may be sucrose, sorbitol, sugar, mannitol, microcrystalline cellulose, starches, calcium carbonate, sodium chloride, lactose, calcium phosphate, calcium sulfate, or sodium phosphate. Examples of buffering agents include citric acid, acetic acid, lactic acid, hydrogenophosphoric acid, and diethylamine. Suitable suspending agents are, for example, naturally occurring gums (e.g., acacia, arabic, xanthan, and tragacanth gum), celluloses (e.g., carboxymethyl-, hydroxyethyl-, hydroxypropyl-, and hydroxypropylmethyl-cellulose), alginates and chitosans. Examples of dispersing or wetting agents are naturally occurring phosphatides (e.g., lecithin or soybean lecithin), condensation products of ethylene oxide with fatty acids or with long chain aliphatic alcohols (e.g., polyoxyethylene stearate, polyoxyethylene sorbitol monooleate, and polyoxyethylene sorbitan monooleate).

Preservatives may be added to the formulations and pharmaceutical compositions comprising any of the rsD4DIIIs and rsZDIIIs variants described herein to prevent microbial contamination that can affect the stability of the formulation and cause infection in the patient. Suitable examples of preservatives include parabens (such as methyl, ethyl, propyl, p-hydroxybenzoate, butyl, isobutyl, and isopropylparaben), potassium sorbate, sorbic acid, benzoic acid, methyl benzoate, phenoxyethanol, bronopol, bronidox, MDM hydantoin, iodopropynyl butylcarbamate, benzalconium chloride, cetrimide, and benzylalcohol. Examples of chelating agents include sodium EDTA and citric acid.

Examples of emulsifying agents are naturally occurring gums, naturally occurring phosphatides (e.g., soybean lecithin; sorbitan mono-oleate derivatives), sorbitan esters, monoglycerides, fatty alcohols, and fatty acid esters (e.g., triglycerides of fatty acids). Anti-foaming agents usually facilitate manufacture, they dissipate foam by destabilizing the air-liquid interface and allow liquid to drain away from air pockets. Examples of anti-foaming agents include simethicone, dimethicone, ethanol, and ether.

Examples of gel bases or viscosity-increasing agents are liquid paraffin, polyethylene, fatty oils, colloidal silica or aluminum, glycerol, propylene glycol, carboxyvinyl polymers, magnesium-aluminum silicates, hydrophilic polymers (such as, for example, starch or cellulose derivatives), water-swellable hydrocolloids, carragenans, hyaluronates, and alginates. Ointment bases suitable for use in the practice of the present invention may be hydrophobic or hydrophilic, and include paraffin, lanolin, liquid polyalkylsiloxanes, cetanol, cetyl palmitate, vegetable oils, sorbitan esters of fatty acids, polyethylene glycols, and condensation products between sorbitan esters of fatty acids, ethylene oxide (e.g., polyoxyethylene sorbitan monooleate), and polysorbates.

Examples of humectants are ethanol, isopropanol glycerin, propylene glycol, sorbitol, lactic acid, and urea. Suitable emollients include cholesterol and glycerol. Examples of skin protectants include vitamin E, allatoin, glycerin, zinc oxide, vitamins, and sunscreen agents.

Formulations and pharmaceutical compositions comprising any of the rsD4DIIIs and rsZDIIIs variants described herein may, alternatively or additionally, comprise other types of excipients including, thickening agents, bioadhesive polymers, and permeation enhancing agents.

Thickening agents are generally used to increase viscosity and improve bioadhesive properties of pharmaceutical compositions. Examples of thickening agents include, but are not limited to, celluloses, polyethylene glycol, polyethylene oxide, naturally occurring gums, gelatin, karaya, pectin, alginic acid, and povidone. Particularly interesting are thickening agents with thixotropic properties (i.e., agents whose viscosity is decreased by shaking or stirring). The presence of such an agent in a composition allows the viscosity of the composition to be reduced at the time of administration to facilitate its local application and, to increase after application so that the composition remains at the site of administration.

Bioadhesive polymers are useful to hydrate skin or mucosa and enhance its permeability. Bioadhesive polymers can also function as thickening agents. Examples of bioadhesive polymers include, but are not limited to, pectin, alginic acid, chitosan, polysorbates, poly(ethyleneglycol), oligosaccharides and polysaccharides, cellulose esters and cellulose ethers, and modified cellulose polymers.

Permeation enhancing agents are vehicles containing specific agents that affect the delivery of active components through the skin/mucosa. Permeation enhancing agents are generally divided into two classes: solvents and surface active compounds (amphiphilic molecules). Examples of solvent permeation enhancing agents include alcohols (e.g., ethyl alcohol, isopropyl alcohol), dimethyl formamide, dimethyl sulfoxide, 1-dodecylazocyloheptan-2-one, N-decyl-methylsulfoxide, lactic acid, N,N-diethyl-m-toluamide, N-methylpyrrolidone, nonane, oleic acid, petrolatum, polyethylene glycol, propylene glycol, salicylic acid, urea, terpenes, and trichloroethanol. The surfactant permeation enhancing agent may be nonionic, amphoteric, cationic, or zwitterionic. Suitable nonioinic surfactants include poly(oxyethylene)-poly(oxypropylene) block copolymers, commercially known as poloxamers; ethoxylated hydrogenated castor oils; polysorbates, such as Tween 20 or Tween 80. Amphoteric surfactants include quaternized imidazole derivatives, cationic surfactants include cetypyridinium chloride, and zwitterionic surfactants include the betaines and sulfobetaines.

Controlled Release of of the Formulations and Pharmaceutical Compositions Comprising any of the rsD4DIIIs and rsZDIIIs Variants Described Herein

In certain embodiments, formulations and pharmaceutical compositions comprising any of the rsD4DIIIs and rsZDIIIs variants described herein are formulated to provide a local controlled release of one or more of the active components. Any pharmaceutically acceptable carrier vehicle or formulation suitable for local administration may be employed. Slow release formulations known in the art include coated-pellets, polymer formulations (such as vesicles or liposomes), microparticles (e.g., microspheres or microcapsules).

A wide variety of biodegradable materials may be used to provide controlled release. The controlled release material should be biocompatible and be degraded, dissolved or absorbed in situ in a safe and pharmaceutically acceptable manner so that the material is removed from the site of administration by natural tissue processes and in a suitable amount of time (e.g., less than one year, less than 6 months, and less than one month, less than one week, less than one day or less than a few hours). The controlled release carrier should not cause any unwanted local tissue reaction, nor should it induce systemic or local toxicity.

Suitable controlled release biodegradable polymers for use in the formulation of the compositions of the invention may comprise polylactides, polyglycolides, poly(lactide-co-glycolides), polyanhydrides, polyorthoesters, polycaprolactones, poly-saccharides, poly-phosphazenes, proteinaceous polymers and their soluble derivatives (such as gelation biodegradable synthetic polypeptides, alkylated collagen, and alkylated elastin), soluble derivatives of polysaccharides, polypeptides, polyesters, and polyorthoesters.

The pharmacokinetic release profile of these formulations may be first order, zero order, bi- or multi-phasic, to provide the desired therapeutic effect over the desired period of time. A desired release profile can be achieved by using a mixture of polymers having different release rates and/or different percents loading of the component(s) of the composition. Methods for the manufacture of coated-pellets, liposomes, microspheres and microcapsules are well known in the art.

Dosage

Administration of formulations and pharmaceutical compositions comprising any of the rsD4DIIIs and rsZDIIIs variants described herein will be in a dosage such that the amount delivered is effective for the intended purpose. The route of administration, formulation and dosage administered will be dependent upon the nature, size, and location of the particular site to be treated, the severity of the infection if already present, the presence of any infection, the age, weight and health condition of the patient as well as upon the potency, bioavailability, and in vivo half-life of the composition used. These factors are readily determinable by the attending physician in the course of the therapy. Alternatively or additionally, the dosage to be administered can be determined from studies using animal models for the particular type of site/disease to be treated. The total dose required for each treatment may be administered by multiple doses or in a single dose. Adjusting the dose to achieve maximal efficacy based on these or other methods are well known in the art and are within the capabilities of trained physicians. As studies are conducted, further information will emerge regarding the appropriate dosage levels and duration of treatment for various stages of the Dengue or Zika virus infection.

Administration

The rsD4DIIIs and rsZDIIIs vaccine or composition may be administered per se or as a pharmaceutical composition, admixed with a pharmaceutically acceptable carrier or excipient. The rsD4DIIIs and rsZDIIIs vaccine or composition can be administered to a subject using any suitable means. In general, suitable means of administration include, but are not limited to, topical, oral, parenteral (e.g., intravenous, subcutaneous or intramuscular), rectal, intracisternal, intravaginal, intraperitoneal, ocular or nasal routes.

Depending on the mode of administration, the rsD4DIIIs and rsZDIIIs vaccine or composition thereof may be in the form of liquid, solid, or semi-solid dosage preparations. For example, the compositions may be formulated as solutions, dispersions, suspensions, emulsions, mixtures, lotions, liniments, jellies, ointments, creams, pastes, gels, hydrogels, aerosols, sprays, microcapsules, microspheres, nanoparticles, pellets, agarose or chitosan beads, capsules, granules, granulates, powders, plasters, bandages, sheets, foams, films, sponges, dressings, drenches, bioadsorbable patches, sticks, delivery devices and implants.

For topical administration, the rsD4DIIIs and rsZDIIIs vaccine or composition thereof may be formulated as solutions, suspensions, dispersions, ointments, creams, pastes, or gel.

The mode of administration of formulations and pharmaceutical compositions comprising any of the rsD4DIIIs and rsZDIIIs variants described herein will mainly depend on the form of the preparation chosen. For example, gels, lotions, creams and ointments may be manually applied or sprayed (either with a manually-activated pump or with the aid of a suitable pharmaceutically acceptable propellant) onto the surface area in need of treatment. Alternatively, a brush, syringe, spatula or a specifically designed container (such as a tube with a narrow tip) can be used to apply the preparation.

In some embodiments, the rsD4DIIIs and rsZDIIIs vaccine or composition for administration to a subject can be formulated for administration by any routine route of administration, including but not limited to, subcutaneous, intra-muscular, intra-nasal, mucosal administration, intravenous.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

An attractive strategy for next-generation platforms or boosting agents is the use of resurfaced subunit vaccines (immunogens) that contain all or parts of the Dengue virus glycoprotein subunit E. The use of recombinant protein immunogens prevents the premature clearance of attenuated or chimeric virus boosters due to pre-existing immunity to vaccine vector components; such clearance would render the boost less effective. Furthermore, subunit vaccines are safer and have lower risk of inducing infection-related side effects because they are replication incompetent.

The major challenge to the use of the DENV EDIII or ZIKV EDIII as a single immunogen is that the immunodominant regions are focused on non-productive or narrow-spectrum epitopes. Protein engineering was used to identify, resurface, and characterize DENV-4 EDIII and ZIKV EDIII variants that overcome these limitations. Furthermore, by re-engineering DENV-4 EDIII and ZIKV EDIII to elicit antibodies targeting a bNAb epitope, the present strategy achieves a single component immunogen that elicits protective antibodies, avoiding the manufacturing and possible safety concerns of multivalent immunogen production. The present application discloses protein immunogens based on resurfaced DENV-4 EDIII and ZIKV EDIII.

Major advances in combinatorial and computational protein design have previously permitted engineering of proteins with enhanced function. “Synthetic protein engineering”—defined here as use of phage- or yeast-displayed libraries with restricted diversity elements encoded by designed, synthetic oligonucleotides—in particular has been used in a variety of applications (32-38) (Chen, G., et al. (2014) ACS Chemical Biology 9, 2263-2273; Koellhoffer, J. F., et al. (2012) Chembiochem 13, 2549-2557; Liu, Y. et al. (2011) Biochemical and biophysical research communications 413, 611-615; Sidhu, S. S., et al. (2006) Nature chemical biology 2, 682-688; Welch, B. D., et al. (2014) Journal of virology 88, 11713-11725; Koide, S., et al. (2009) ACS chemical biology 4, 325-334; Wojcik, J., et al. (2010) Nature structural & molecular biology 17, 519-527). The combination of highly specified libraries, coupled with complete control of the binding selections, provides the opportunity to develop reagents that have either enhanced specificity for a single target (e.g., for particular post-translational modifications) or multi-specificity without being non-specific. This method has been used to identify highly specific binding antibodies or proteins that generally would not be accessible by other methods (32-34, 36) (Chen, G., et al. (2014) ACS Chemical Biology 9, 2263-2273; Koellhoffer, J. F., et al. (2012) Chembiochem 13, 2549-2557; Liu, Y. et al. (2011) Biochemical and biophysical research communications 413, 611-615; Welch, B. D., et al. (2014) Journal of virology 88, 11713-11725). For example, specific fusogenic intermediates of virus glycoproteins have been targeted by this approach.

A compact cross-reactive epitope among DENV1 and ZIKV EDIII has been described, targeted by a single human mAb, but otherwise there are no shared epitopes among DENV and ZIKV in the EDIII regions. This contrasts with shared epitopes in other regions of the E glycoprotein (e.g., the fusion loop in EDII). Inclusion of such cross-reactive epitopes increases the possibility of inducing a cross-reactive but non-neutralizing response by vaccination. Since the present studies focused on minimal EDIII-based structural design, the risk of ADE across DENV and ZIKV is minimized, yielding a potential advantage over live virus or subunit vaccines encoding most or all of the prM and E glycoproteins.

### Example 1: Resurfaced DENV-4 EDIIIs (“rsD4DIIIs”)

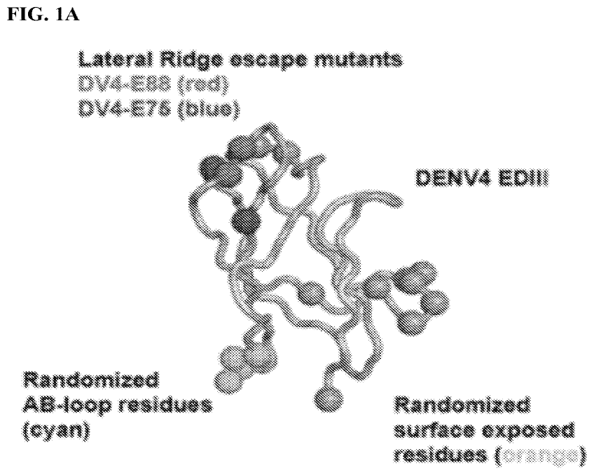

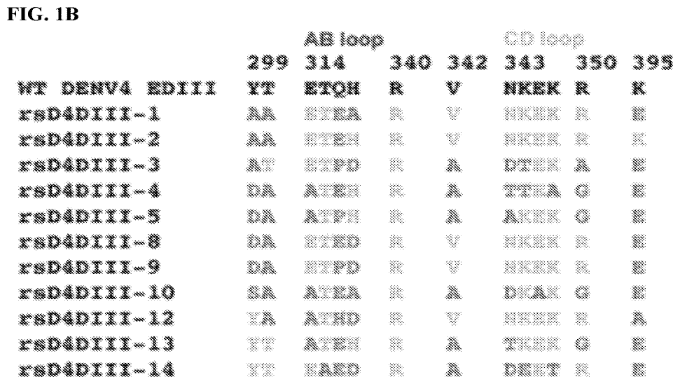

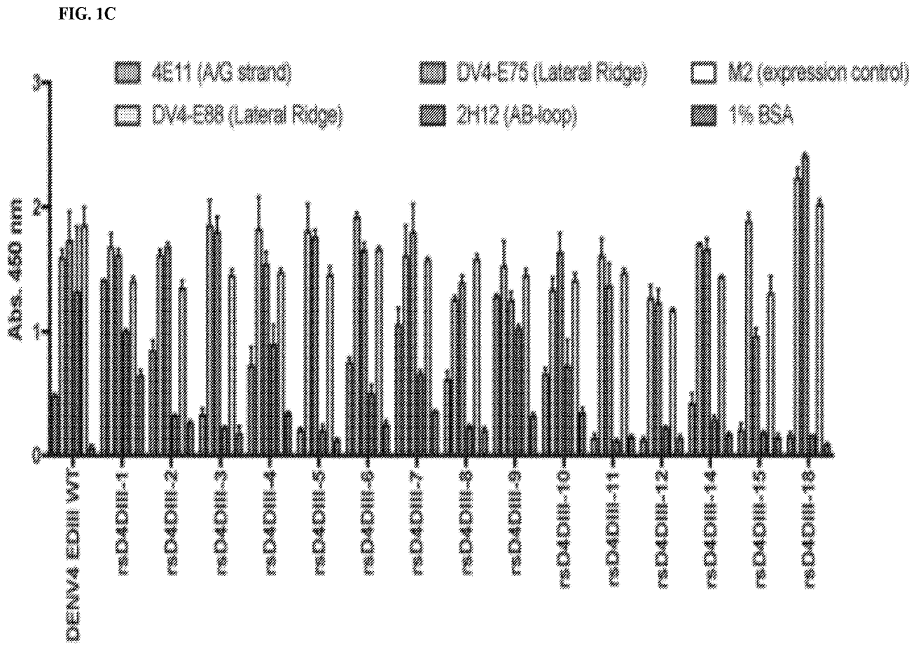

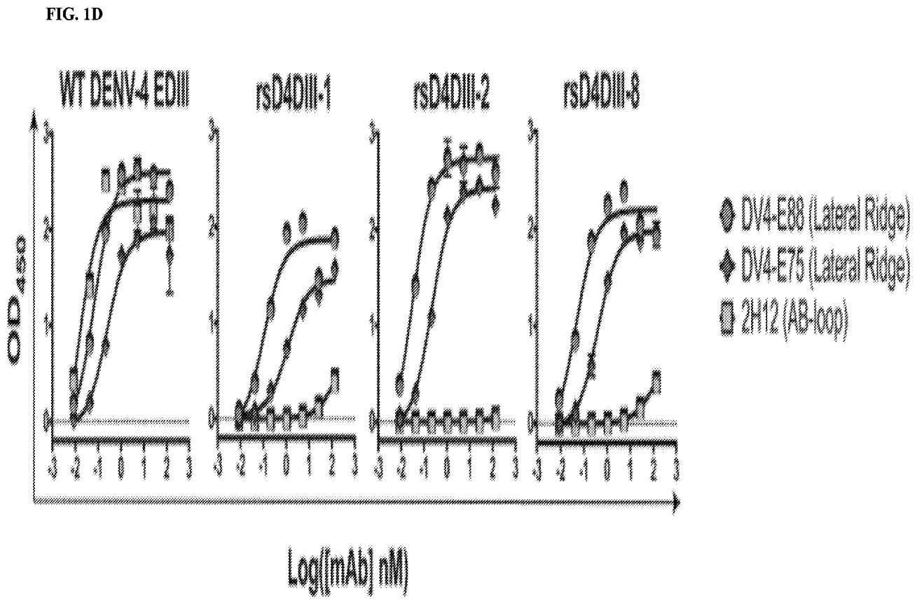

In the present studies, DENV4 EDIII-based immunogens were developed which preserved potently neutralizing lateral ridge (LR) epitopes, but contain alterations in other surface exposed regions such as the AB- and CD-loop. This DENV4-specific EDIII immunogen could later be combined with rsDIII-Ala30 to provide broad protection against DENV1-4 (rsDIII-Ala30 is described in PCT/US17/17637 (WO2017/142831); herein incorporated by reference in its entirety). A resurfacing phage display library was generated based on the DENV4 EDIII scaffold, in which surface-exposed residues on AB-loop, CD-loop, D-strand, and other regions were permitted to vary in the WT/Ala restricted diversity scheme (FIG. 1A). This library was subjected to selection against two DENV4 type-specific murine mAbs, DV4-E88 and DV4-E75. The mAbs DV4-E88 and DV4-E75 were previously isolated by the Diamond laboratory and potently neutralize strains from multiple DENV4 genotypes (Sukupolvi-Petty, S. et al. (2013) J of virology 87, 8826-8842). Although X-ray structures are not available for either mAb, viral escape and mutational mapping studies have localized their binding epitopes within the LR; specifically the BC- and DE-loops (DV4-E88), and B-strand and DE-loop (DV4-E75). Furthermore, DV4-E88 was shown to confer protection against viral challenge in an immunocompromised mouse model. It was reasoned that “resurfaced DENV4 EDIII variants” (“rsD4DIIIs”) that are recognized by DV4-E75 and DV4-E88, but not by non-neutralizing mAb 2H12 (AB-loop), would maintain DENV4-specific neutralizing epitopes and thus potentially act as DENV4-specific immunogens. FIG. 1B and Table 1 shows the sequences of 11 of 15 representative rsD4DIIIs isolated. FIG. 1C shows phage ELISA against DV4-E88 and DV4-E75 (LR neutralizing mAbs), 2H12, expression control (M2), and negative control wells (BSA) for all 15 rsD4DIII selectants. The rsD4DIIIs exhibit diverse sequences in the randomization regions but all maintain a strong binding signal against the DENV4 neutralizing mAbs. Importantly, rsD4DIII-2, -3, -5, -8, -11, -12, -14, 15, and -18 do not bind 2H12, suggesting that the AB-loop has been masked. Similar to the WT DENV4 EDIII, moderate or no binding to 4E11 was observed for most of the rsD4DIIIs.

TABLE 1
Sequences of Selected rsD4DIII Clones.

| | AB Loop | | | | | | CD loop | | | | | | | |
|---|---|---|---|---|---|---|---|---|---|---|---|---|---|---|
| | 299 | 300 | 314 | 315 | 316 | 317 | 340 | 342 | 343 | 344 | 345 | 346 | 350 | 395 |
| WT | Y | T | E | T | Q | H | R | V | N | K | E | K | R | K |
| 1 | A | A | E | T | [symbol] | A | R | V | N | K | E | K | R | [symbol] |
| 2 | A | A | E | T | [symbol] | H | R | V | N | K | E | K | R | K |
| 3 | A | T | E | T | [symbol] | [symbol] | R | A | [symbol] | [symbol] | E | K | A | [symbol] |
| 4 | [symbol] | A | A | T | [symbol] | H | R | A | [symbol] | [symbol] | E | A | [symbol] | [symbol] |
| 5 | [symbol] | A | A | T | [symbol] | H | R | A | A | K | E | K | [symbol] | [symbol] |
| 8 | [symbol] | A | E | T | [symbol] | [symbol] | R | V | N | K | E | K | R | [symbol] |
| 9 | [symbol] | A | E | T | [symbol] | [symbol] | R | V | N | K | E | K | R | [symbol] |
| 10 | [symbol] | A | A | T | [symbol] | [symbol] | R | A | [symbol] | K | A | K | [symbol] | [symbol] |
| 12 | Y | A | A | T | [symbol] | [symbol] | R | V | N | K | E | K | R | A |
| 13 | Y | T | A | T | [symbol] | H | R | A | [symbol] | K | E | K | [symbol] | [symbol] |
| 14 | Y | T | E | A | [symbol] | [symbol] | R | A | [symbol] | [symbol] | E | [symbol] | R | [symbol] |

Residues bolded and underlined indicate mutation from WT to Ala. Residues bolded and italicized indicate mutation to from WT to another residue identity. The amino acid residues in Table 1 correspond to the underlined portions of the corresponding parts of the amino acid sequences of the variants disclosed herein above: rsD4DIII-1 (“1” in Table 1) (SEQ ID NO:3) or (SEQ ID NO:69), rsD4DIII-2 (“2” in Table 1) (SEQ ID NO:4) or (SEQ ID NO:71), rsD4DIII-3 (“3” in Table 1) (SEQ ID NO:5), rsD4DIII-4 (“4” in Table 1) (SEQ ID NO:6) or (SEQ ID NO:73), rsD4DIII-5 (“5” in Table 1) (SEQ ID NO:7), rsD4DIII-8 (“8” in Table 1) (SEQ ID NO:8) or (SEQ ID NO:77), rsD4DIII-9 (“9” in Table 1) (SEQ ID NO:9) or (SEQ ID NO:79), rsD4DIII-10 (“10” in Table 1) (SEQ ID NO:10) or (SEQ ID NO:81), rsD4DIII-12 (“12” in Table 1) (SEQ ID NO:11), rsD4DIII-13 (“13” in Table 1) (SEQ ID NO:12), or rsD4DIII-14 (“14” in Table 1) (SEQ ID NO:13). The wildtype DENV-4 EDIII amino acid sequence is shown in SEQ ID NO:1.

The rsD4DIII candidates were characterized, ranked for favorable biochemical binding and stability profiles, and tested for immunogenicity and challenge studies.

Binding characteristics. rsD4DIII-2, -4, -7, -8, -9, -10, -11, and -18 were produced in E. coli and rescued from inclusion bodies. As with DENV2 EDIII variants described in PCT/US17/17637 (WO2017/142831) (herein incorporated by reference in its entirety), the monomeric species were isolated by size-exclusion chromatography (SEC) to avoid eliciting aggregate-specific rsD4DIII antibody responses that are unlikely to target conformational epitopes such as the A/G strand or LR. The binding profiles of purified rsD4DIIIs toward mAbs were confirmed by enzyme-linked immunosorbent assay (ELISA) and β-Lactamase inhibitor (BLI). The binding characteristics of the rsD4DIIIs were determined. The rsD4DIIIs were tested for expression yield and stability upon refolding. The rsD4DIIIs were further assayed for immunogenicity and in NMR studies.

Immunogenicity and challenge experiments. The ability of rsD4DIIIs to elicit DENV4 neutralizing antibody were examined. Groups of male and female BALB/c mice (n=10) were immunized and boosted twice with 20 μg of WT DENV4 EDIII or rsD4DIIIs in CFA/IFA. An additional control group receiving adjuvant only will be included. In some experiments, the adjuvant was Freund's adjuvant. While Freud's adjuvant is not approved, it has been described in previous studies and literature reports in mice. Adjuvants that are appropriate for use in non-human primates or humans were also explored for the nanoparticles. Mice were bled by tail vein on days 0, 15, 30, 60, and 90. The reactivity of the sera toward WT EDIII from DENV1, 2, 3, and 4 were examined, as well as the capacity to neutralize four different DENV strains corresponding to each serotype (DENV-1 WestPac 74, DENV-2 D2S10, DENV-3 C0360/94, or DENV-4 TVP-376) (Sarathy, V. V. et al. (2015) J of Virol 89, 1254-1266; Sarathy, V. V. et al. (2015) J Gen Virol 96, 3035-3048; Shrestha, B. et al. (2010) PLoS Pathog 6, e1000823; Shresta, S. et al. (2006) J of Virol 80, 10208-10217). It was expected that most of the neutralization profiles to be DENV4-specific, as per design. To further explore the specific epitopes targeted by serum antibodies, the sera were further be tested for reactivity toward mutants DENV4 EDIII variants in which LR residues are altered and have been shown previously to abrogate mAb binding for DV4-E88 and DV4-E75. These include A331G and T361A for DV4-E88; and K325A, N360A, and T361 for DV4-E75 (Sukupolvi-Petty, S. et al. (2013) J of Virol 87, 8826-8842).

The inventors tested strongly neutralizing sera for protective capacity in a lethal DENY challenge mouse model, as previously described (Sukupolvi-Petty, S. et al. (2013) J of Virol 87, 8826-8842)28. The rsD4DIII immune sera were passively transferred into AG129 mice (which lack type I and III interferon signaling) and challenged in parallel with a lethal dose of DENV4. Decreasing amounts of pooled serum (e.g., 100, 10, or 1 μl) from immunized mice were administered to groups (n=5, at least two independent experiments) of naïve AG129 mice one day prior to lethal challenge. Viremia (days 1 and 3), weight loss, and survival were monitored. These studies established the ability of rsD4DIII immunogens to induce protective antibody responses against DENV4. By performing limited dose response studies, the relative potency of different recombinant immunogens were defined to inform prioritization.

Single B cell sorting. To further explore the effects of resurfacing on nature of immune response, the B cell response of the rsD4DIIIs were examined by single B cell sorting. Populations of splenic memory B cells (MBCs) 90 days post-immunization with rsD4DIIIs were analyzed for their reactivity toward WT DENV4 EDIII-Fc fusion proteins or variants containing null mutations in the LR ridge epitope (A331G/T361A). This technology was used to sort for virus-specific MBCs in the context of infection by West Nile virus, a closely related flavivirus (Purtha, W. E. et al. (2011) The J of Exp Med 208, 2599-2606). If immunization with rsD4DIIIs resulted in a higher number of LR-specific antibodies, then it was predicted that there should be a significantly lower population of B cells that are reactive toward the LR mutants than WT DENV4 EDIII-Fc. The specific activities of individual antibodies were explored by isolation of mAbs from single B cell sorting. RNA from individual sorted cells were recovered as done previously (Purtha, W. E. et al. (2011) The J of Exp Med 208, 2599-2606), and the variable domains of the heavy and light chains were cloned by nested PCR, and then expressed after transient transfection of HEK293 cells (Fernandez, E. et al. (2018) mBio 9; Sapparapu, G. et al. (2016) Nature 540, 443-447). Reactivity of the resulting mAbs toward WT DENV4 EDIII or LR null mutants were examined by ELISA and BLI, and the neutralizing activity tested against all four DENV serotypes.

Reactivity studies with serum samples from DENV patients. To explore the role of EDIII epitopes in protective responses resulting from natural human infection, a large cohort of DENY immune sera were queried for reactivity profiles toward the best rsD4DIIIs as well as rsDIII-Ala30. While not an absolute requirement for advancing an immunogen, the demonstration that natural antibodies arising during infection target a designed immunogen confirms that the structural engineering has preserved the epitope and suggests that antibodies specific for the immunogen can be elicited in naïve humans. One hundred sera were obtained from DENV convalescent patients (up to 50 μL per sample) as well as 25 control samples from flavivirus patient cohorts in Nicaragua (provided by Dr. Eva Harris). All sera were tested for neutralizing titer against DENY and antigen reactivity by ELISA. Since all samples have been deidentified and are provided by a 3rd party, this work does not qualify as Human Subjects Research as per NIH guidelines.

Structural characterization by NMR. The 2-4 rsD4DIIIs that elicited the most potently neutralizing sera during immunogenicity studies were characterized structurally by NMR spectroscopy to ensure that the overall Ig-like fold of the domain has not been disrupted by substitutions. The rsD4DIIIs were produced as either ¹⁵N or ¹⁵N/¹³C labeled sample in E. coli as was performed previously with DENV2-based rsDIIIs as described in PCT/US17/17637 (WO2017/142831) (herein incorporated by reference in its entirety). Initial analysis were focused on comparisons of spectra between WT DENV4 EDIII and rsD4DIIIs to determine the amount of cross-peak overlap. It was expected that most rsD4DIIIs contained a high degree of spectral overlap with WT DENV4 EDIII, indicating similar structures. Once experimental parameters were established, the backbone resonance assignments were completed by the standard triple resonance approach, supplemented by 3D ¹H¹⁵N NOESY-HSQCs to help assign or verify connectivities. Backbone NOEs and chemical shift constraints were used to define the local backbone structure and compared among rsDIIIs and WT DENV2 EDIII. If sufficient rationale exists, the atomic three-dimensional structures of WT DENV2 EDIII and/or rsD4DIIIs will be determined using NMR-derived constraints. This will be performed only in cases where the precise orientation of β-strands and side chain residues relative to one another affects the resurfacing design strategy.

(i) Protocol to improve expression or solubility of rsD4DIIIs. Previous expression protocols for refolding EDIII from inclusion bodies of E. coli have provided milligram quantities of all DENV1-4 WT EDIIIs as well as rsDIII-Ala11 and rsDIII-Ala30. In the event that rsD4DIIIs variants aggregate or express with lower yield than WT, additional rsD4DIIIs variants will be examined for favorable reactivity profiles and aggregation by dynamic light scattering will be assessed. Additional WT/Ser resurfacing librariers can be generated. Ser contains hydrophilic functionality and thus rsD4DIIIs bearing Ser substitutions likely have improved solubility relative to rsD4DIIIs containing Ala mutations. (ii) Protocol to enhance stimulation of strong antibody response for rsD4DIIIs. Although DENV2-based rsDIIIs induce a strong antibody response, it is possible that induction of durable antibody responses against rsD4DIIIs may require further incorporation of an immunodominant CD4+ T cell epitope. If necessary, a 13-amino acid universal Pan HLA-DR Epitope (PADRE) T cell epitope will be incorporated to promote affinity maturation, and formation of memory B cells and long-lived plasma cells 64, 65 (Ghaffari-Nazari, H. et al. (2015) PloS one 10; La Rosa, C. et al. (2012) The J of infectious diseases 205, 1294-1304). Second-generation libraries can also be further designed or screened to resurface a higher percentage of the DENV4 EDIII to remove additional surface characteristics. Additional randomizations screens, such as WT/Ser, can be explored. (iv) Protocol for NMR analysis. If there is insufficient spectral resolution to obtain full crosspeak assignments with rsD4DIIIs, co-crystallization studies will be initiated with antigen binding fragments (Fabs) in complex with rsD4DIIIs. (v) Protocol to monitor for “self”-directed responses. Self-directed responses can be monitored with tissue reactivity studies on sera from immunized mice.

### Example 2: Resurfaced ZIKV EDIIIs (“rsZDIIIs”)

In the present studies, ZIKV EDIII-based immunogens were developed, characterized, assayed, and tested using similar experimentals as described above for resurfaced DENV EDIII.

Characterize and evaluate ZIKV rsDIIIs in mice. ZIKV EDIII is a target of protective neutralizing mAbs isolated from natural human or mouse infections (Zhao, H. et al. (2016) Cell 166, 1016-1027; Stettler, K. et al. (2016) Science (New York, N.Y.) 353, 823-826; Robbiani, D. F. et al. (2017) Cell 169, 597-609; Rogers, T. F. et al. (2017) Sci Immunol 2). Most human ZIKV EDIII mAbs do not cross-react with DENV EDIII with the exception of Z004, Z006, and ZIKV-116, three mAbs that bind the LR epitope and cross-neutralize DENV1, but none of the other DENV serotypes (Robbiani, D. F. et al. (2017) Cell 169, 597-609; Sapparapu, G. et al. (2016) Nature 540, 443-447). The LR epitope encompasses residues at the N-terminus, BC-, DE-, and FG-loops, but the cross-reactive region targeted by Z004 and Z006 is localized to E393 and K394 of the FG-loop (ZIKV numbering; E384/K385 in DENV) (Nybakken, G. E. et al. (2005) Nature 437, 764-769). Aside from this small epitope, most other ZIKV EDIII mAbs do not cross-react with DENY EDIII, in contrast to ZIKV mAbs targeting other E epitopes. Thus, EDIII-directed immunogens are unlikely to elicit ADE responses between DENV and ZIKV. As previously described Dr. Diamond's group isolated murine mAb ZV-67, which is protective in a lethal mouse ZIKV model and binds to the LR epitope (Zhao, H. et al. (2016) Cell 166, 1016-1027). However, mAbs targeting other regions, such as the ABDE sheet (bound by mAb ZV-2) or the C—C′-loop (bound by mAb ZV-64), were non- or less potently neutralizing.

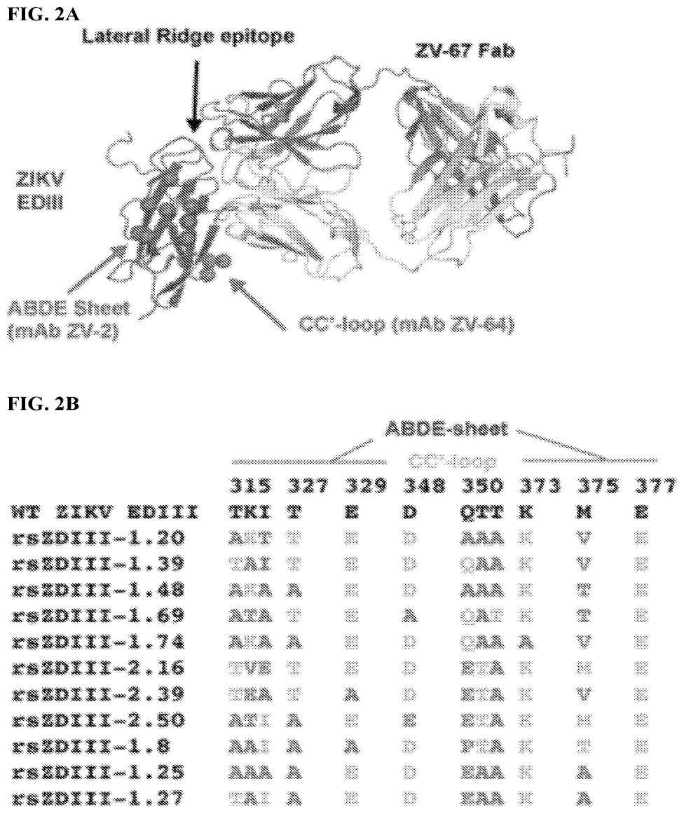

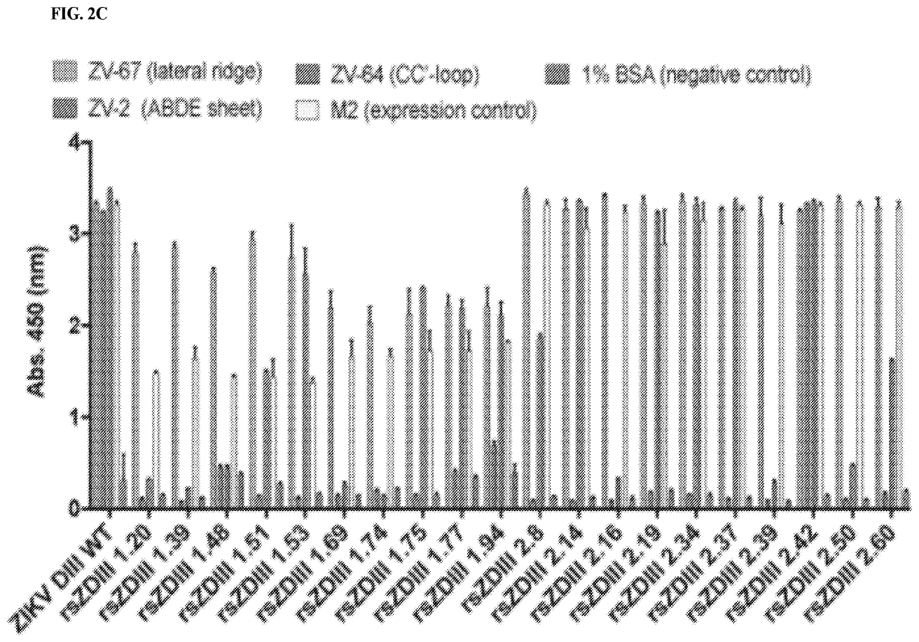

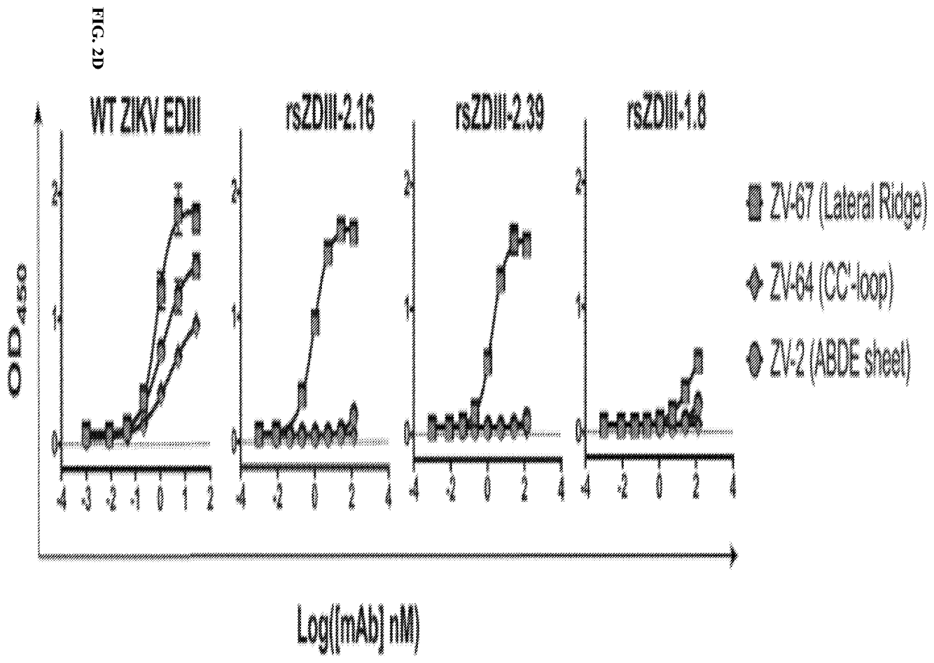

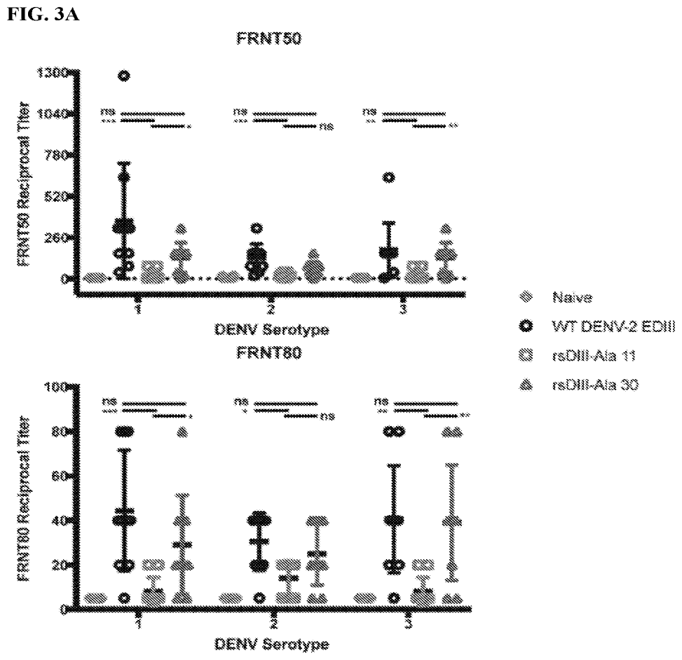

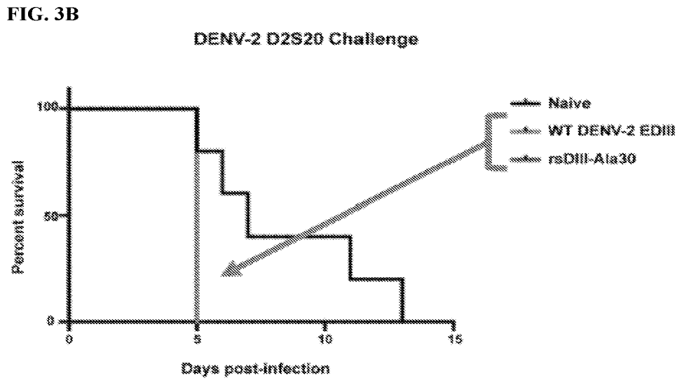

In the present studies, the ZIKV ABDE sheet and CC′-loop were resurfaced by phage display (“rsZDIIIs”), while maintaining the critical LR epitope. The targeted regions were allowed to vary in the WT/Ala combinatorial format and selected for binding to ZV-67, and counter-selected against ZV-2 and ZV-64. Representative sequences for 11 of 18 selected clones are shown in FIG. 2B and Table 2, again containing a high level of substitution in the randomized regions. Phage ELISA (FIG. 2C) indicated that most rsZDIII clones retained reactivity toward ZV-67, but not ZV-2. Some rsZDIII exhibited residual activity toward ZV-64, and thus the C—C′-loop epitope may not have been completely masked in some cases. Using similar experimentals as described for rsD4DIIIs above, the rsZDIIIs were characterized for their ability to elicit neutralizing antibodies. As the experimentals are similar to rsD4DIIIs above, a brief overview of the rsZDIIIs experimentals are provided here, but focused on experimental details that differ.

TABLE 2
Sequences of Selected rsZDIII Clones

| | ABDE-sheet (CC’-loop) | | | | | | | | | | | |
|---|---|---|---|---|---|---|---|---|---|---|---|---|
| | 315 | 316 | 317 | 327 | 329 | 348 | 350 | 351 | 352 | 373 | 375 | 377 |
| WT | T | K | I | T | E | D | Q | T | T | K | M | E |
| 1.20 | A | K | [symbol] | T | E | D | A | A | A | K | [symbol] | E |
| 1.39 | T | A | [symbol] | T | E | D | Q | A | A | K | [symbol] | E |
| 1.48 | A | K | A | A | E | D | A | A | A | K | [symbol] | E |
| 1.69 | A | [symbol] | A | T | E | A | Q | A | T | K | [symbol] | E |
| 1.74 | A | K | A | A | E | D | Q | A | A | A | [symbol] | E |
| 2.16 | T | [symbol] | [symbol] | T | E | D | [symbol] | T | A | K | M | E |
| 2.39 | T | [symbol] | A | T | A | D | [symbol] | T | A | K | [symbol] | E |
| 2.50 | A | [symbol] | I | A | E | [symbol] | [symbol] | T | A | K | M | E |
| 1.8 | A | A | I | A | A | D | [symbol] | T | A | K | T | E |
| 1.25 | A | A | A | A | E | D | [symbol] | A | A | K | A | E |
| 1.27 | T | A | T | A | E | D | [symbol] | A | A | K | A | E |

Residues bolded and underlined indicate mutation from WT to Ala. Residues bolded and italicized indicate mutation to from WT to another residue identity. The amino acid residues in Table 2 correspond to the underlined portions of the corresponding parts of the amino acid sequences of the variants disclosed herein above: rsZDIII-1.20 (“1.20” in Table 2) (SEQ ID NO:18) or (SEQ ID NO:57), rsZDIII-1.39 (“1.39” in Table 2) (SEQ ID NO:19) or (SEQ ID NO:59), rsZDIII-1.48 (“1.48” in Table 2) (SEQ ID NO:20), rsZDIII-1.69 (“1.69” in Table 2) (SEQ ID NO:21), rsZDIII-1.74 (“1.74” in Table 2) (SEQ ID NO:22), rsZDIII-2.16 (“2.16” in Table 2) (SEQ ID NO:23) or (SEQ ID NO:61), rsZDIII-2.39 (“2.39” in Table 2) (SEQ ID NO:24) or (SEQ ID NO:63), rsZDIII-2.50 (“2.50” in Table 2) (SEQ ID NO:25), rsZDIII-1.8 (“1.8” in Table 2) (SEQ ID NO:26), rsZDIII-1.25 (“1.25” in Table 2) (SEQ ID NO:27), or rsZDIII-1.27 (“1.27” in Table 2) (SEQ ID NO:28). The wildtype ZIKV EDIII amino acid sequence is shown in SEQ ID NO:16.

Binding characteristics. Since all 18 rsZDIII candidates have favorable reactivity profiles, small scale expression was performed of all 18 candidates to determine expression yields and solubility. The binding profiles of purified rsZDIIIs were characterized by ELISA and BLI, immunogenicity assays, and NMR studies as described above.

Immunogenicity and challenge experiments. Initial immunogenicity experiments were performed similarly to the procedures described above for BALM mice, except that sera were tested for the capacity to bind ZIKV EDIII and neutralize ZIKV infection. Those rsZDIIIs that elicited strongly neutralizing sera were tested for their capacity to induce protective responses in mice. The Diamond laboratory described a lethal model for ZIKV infection with an adapted African strain in which WT C57BL/6 mice are rendered IFN-deficient only at the time of challenge by treating with a blocking anti-IFNAR1 mAb immediately preceding ZIKV challenge (Zhao, H. et al. (2016) Cell 166, 1016-1027). Mice challenged in this way succumb to ZIKV infection within 12 days. Since this model utilized immunocompetent WT mice, which are fully capable of developing serum antibody and memory B cell responses, vaccine challenge experiments do not require a passive transfer step as described for DENV. The Diamond laboratory has used this model successfully to test modified mRNA and live-attenuated vaccine candidates against ZIKV in male and pregnant female mice (Richner, J. M. et al. (2017) Cell 169, 176; Richner, J. M. et al. (2017) Cell 170, 273-283; Shan, C. et al. (2017) Nat Commun 8, 676). The ZIKV EDIIIs provided herein were used to vaccinate and boost C57BL/6 mice. Subsequently, these cohorts were treated with the anti-IFNAR mAb, at day −1 to infection, and challenged with ZIKV. Survival and weight loss were the primary endpoints. Viremia was used as a secondary endpoint as required. ZIKV rsDIIIs were designed based on the Asian/American lineage, but heterologous protection against the African strain would imply broad activity. The Diamond laboratory has recently inserted the adaptive mutation of the African strain (a single NS4B (G18R) amino acid change) into infectious clones of ZIKV-Brazil and ZIKV-French Polynesia to enhance virulence in mice (Gorman, M. J. et al. (2018) Cell host & microbe 23, 672-685). These strains were tested in confirmatory studies. Protection studies were performed during pregnancy to confirm that rsZDIIIs protected against congenital ZIKV syndrome in mice.

Single B cell sorting. B cell responses were profiled using FACS as described above with WT and LR mutant ZIKV DIII-Fc fusion proteins. Individual mAbs will be isolated, expressed, and characterized as described above.

Reactivity studies with serum samples with ZIKV patients. Human ZIKV mAbs have been shown to target EDIII (Stettler, K. et al. (2016) Science (New York, N.Y.) 353, 823-826). To determine whether EDIII LR responses are generated in humans, serum reactivity studies were performed with the best 1-2 ZIKV rsDIIIs. As described above, Dr. Eva Harris supplied 100 ZIKV serum samples from convalescent patients in her cohort.

Structural characterization by NMR. The 2-4 most promising rsZDIIIs variants were characterized by NMR in comparison to WT ZIKV EDIII, as described above.

### Example 3: Engineer and Evaluate Multivalent, Broad Flavivirus Resurfaced EDIII Nanoparticles

Although rsDIIIs elicited cross-neutralizing responses, there was some variability in the magnitude of the response in different mice (PCT/US17/17637 (WO2017/142831; herein incorporated by reference in its entirety). In particular, while some mice that received rsDIII-Ala30 immunization had strong neutralizing titers, others in the same group developed weak responses. Furthermore, although the neutralizing titers observed were within the range of previous studies, the pooled sera from the rsDIII-Ala30 immunized mice were not potent enough to protect AG129 mice from viral challenge by DENV2 at a dose of 100 μL ((PCT/US17/17637 (WO2017/142831; herein incorporated by reference in its entirety)). Therefore, provided herein are studies determining the capacity of multivalent protein nanoparticles bearing rsDIIIs to elicit more potently neutralizing and protective responses. Nanoparticle-presented immunogens can improve titers, likely due to their capacity to crosslink B cell receptors which promotes expansion and affinity maturation (Tokatlian, T. et al. (2019) Science (New York, N.Y.) 363, 649-654; Marcandalli, J. et al. (2019) Cell 176, 1420-1431). In the case of rsDIIIs, such improvement is likely sufficient to provide protection in mice and humans. Such improvement is likely sufficient to also provide protection in any mammal.

The studies herein utilized the well-characterized nanoparticle platforms of Aquifex aeolicus Lumazine Synthase (“aaLS”) and H. pylori ferretin (“hpFer”). The aaLS platform is a 16 kDa enzyme involved in riboflavin biosynthesis that assembles into 60-mer nanoparticles of ˜15 nm diameter. The aaLS-based nanoparticles have been used previously as a carrier for immunogen presentation of HIV-1 gp120 (Jardine, J. et al. R (2013) Science (New York, N.Y.) 340, 711-716). The hpFer, 20 kDa, assembles into a 24-mer nanoparticle (10 nm diameter). The hpFer has previously been used for presentation of influenza and Epstein Barr Virus immunogens (Kanekiyo, M. et al. (2015) Cell 162, 1090-1100; Kanekiyo, M. et al. (2013) Nature 499, 102-106). Since expression and purification of EDIII proteins requires refolding from inclusion bodies, rsD4DIIIs and rsZDIIIs variants were generated separately from nanoparticles and conjugated using the Spycatcher/Spytag system (Zakeri, B. et al. (2012) PNAS 109, E690-697). The 151-residue protein CnaB2 contains a natural isopeptide bond that can be reconstituted as two polypeptides. The “Spytag” is a 13-residue peptide corresponding to the C-terminal β-strand of CnaB that spontaneously forms an isopeptide bond to its protein partner, Spycatcher, which consists of the remainder of the domain. Thus, the rsD4DIIIs and rsZDIIIs variants conjugated to nanoparticle compositions herein leverage the Spycatcher/Spytag system by utilizing Spytagged-rsD4DIII variants or Spytagged-rsZDIII variants as “building blocks” for facile antigen presentation on different nanoparticle scaffolds.

When used in the vaccination studies, the rsD4DIIIs and rsZDIIIs variants were engineered with a C-term SpyTag sequence to facilitate aaLS nanoparticle conjugation. The SpyTag Amino acid Sequence is GSGSMAHIVMVDAYKPTK (SEQ ID NO: 65). The SpyTag Nucleotide Sequence is

(SEQ ID NO: 66)
GGTTCTGGTTCTATGGCTCACATCGTTATGGTTGACGCTTACAAACCGACC
AAA.

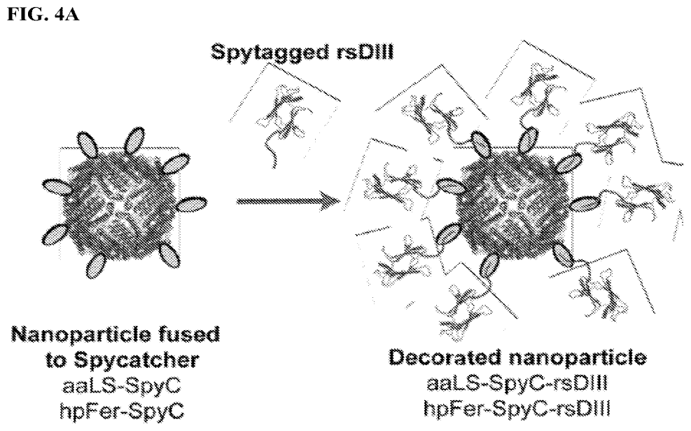

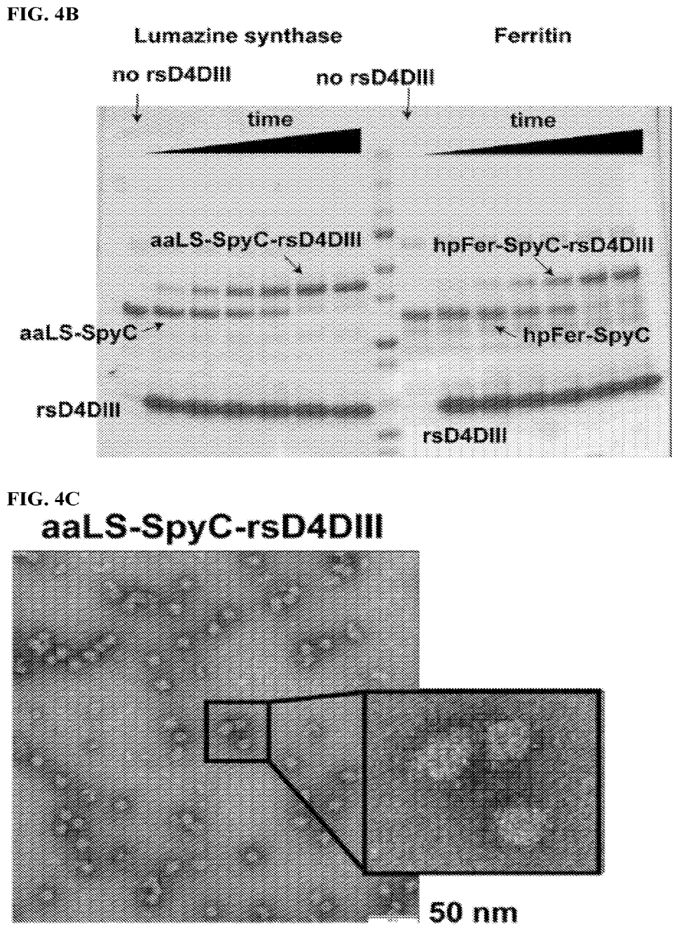

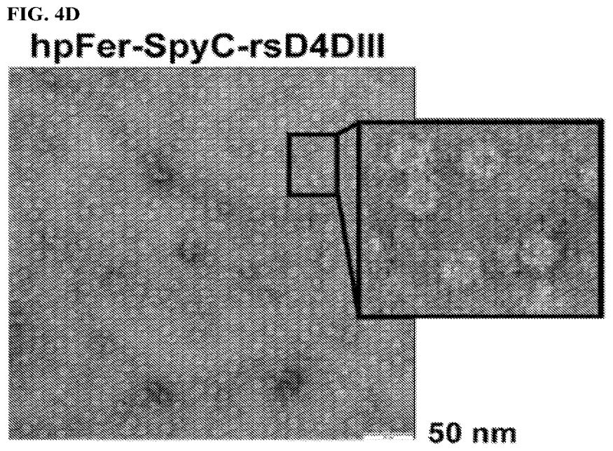

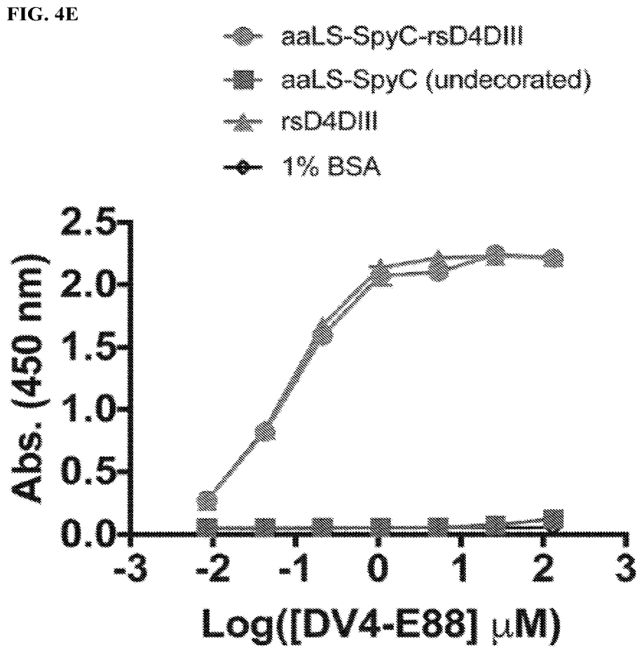

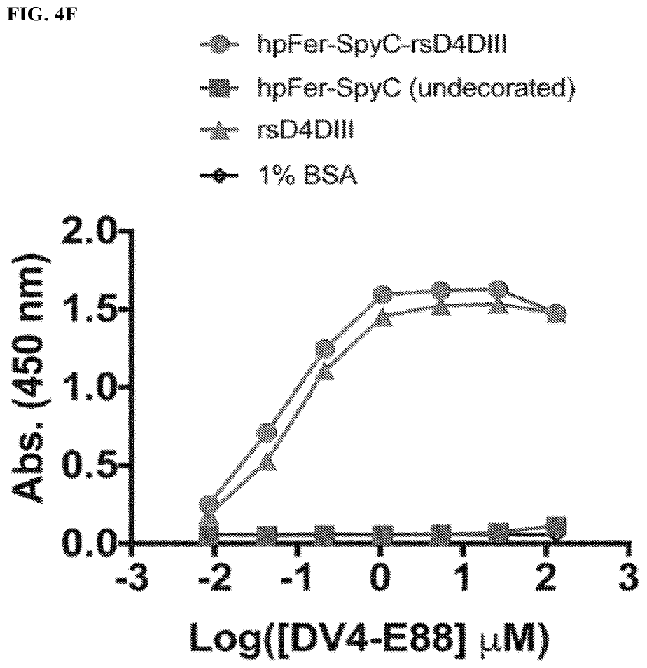

The rsD4DIIIs and rsZDIIIs variants containing the Spytag were generated and conjugated to aaLS or hpFer nanoparticles that contain Spycatcher on the surface (FIG. 4A). Conjugation of an rsD4DIII to both aaLS-SpyC and hpFer-SpyC proceeded efficiently (FIG. 9B) and yielded monodisperse particles (FIGS. 4C and 4D). Both rsD4DIII particles are recognized by DV4-E88 (DENV4-specific neutralizing mAb) (FIGS. 4E and 4F), indicating that the rsD4DIII is presented in an appropriate conformation.

Purification Protocols.

rsD4DIIIs and rsZDIIIs variants expression. From a freshly transformed plate, incubate 1 colony in 50 mL 2×YT media with 50 μL carbenicillin overnight at 37° C., 220 RPM. Transfer 5 mL overnight culture to 100 mL low phosphate media with 100 μL carbenicillin; make up to ten 100 mL cultures. Incubate for 24 hours at 30° C., 220 RPM. Harvest cells via centrifugation at 4,500 RPM, 4° C., for 15 minutes. Weigh cell pellets and freeze at −20° C. until purification.

rsD4DIIIs and rsZDIIIs variants purification. Thaw cells at room temperature. Per gram of wet cell weight add 5 mL of 1×-diluted Bug Buster. Resuspend cells in PBS (20 mM sodium phosphate monobasic+150 mM NaCl) with EDTA-free protease cocktail inhibitor and DNaseI. Add 10× Bug Buster to dilute to 1× and incubate with gentle rocking for 20 minutes at room temperature. Centrifuge at 12,000 RPM (ss-34), for 30 minutes at 4° C. Rinse the pellet (inclusion body fraction) with PBS by vortexing and centrifuge for 30 minutes at 12,000 RPM and 4° C. Discard supernatant. Resuspend pellet in 8M urea/PBS overnight with stirring. Spin down at 15° C. for 30 minutes at 12,000 RPM. Keep the supernatant (solubilized inclusion body).

Wash 1 mL Ni-NTA beads (Qiagen) with 8M urea/PBS. Load inclusion body fraction onto the column and collect the flow through. Wash with 7.5 mL 8M urea/PBS, pH 6.0 and collect fraction. Wash with 7.5 mL 8M urea/PBS, pH 5.3/55 mM Imidazole and collect fraction. Elute with 3.8 mL 8M urea/PBS, pH 4.0/250 mM Imidazole and collect elute. Elute with 5 mL 8M urea/PBS, pH 4.0/500 mM Imidazole and collect elute. Run SDS-PAGE to verify purity and pool relevant fractions.

rsD4DIIIs and rsZDIIIs variants refolding. Dilute denatured rsD4DIIIs and rsZDIIIs variants 20-fold into 20 mM Tris-HCl, 500 mM NaCl, pH 7.8— results in a final urea concentration of 0.4M. Dialyze sample in 20 mM Tris-HCl, 500 mM NaCl, pH 7.8 to remove urea.

rsD4DIIIs and rsZDIIIs variants purification and refolding on column. Prepare inclusion body fraction as above and wash 1 mL Ni-NTA beads as above. Load inclusion body fraction onto column and collect the flow through. Wash with 5 mL 8M urea/PBS, pH 6.0 and collect fraction. Wash with 5 mL 8M urea/PBS, pH 5.8 and collect fraction. Wash with 10×1 mL refolding buffer (20 mM Tris-HCl, 500 mM NaCl, pH 7.8) and collect fraction. Elute with 5 mL 20 mM Tris/500 mM NaCl/250 mM Imidazole, pH 7.8 and collect elute. Elute with 5 mL 20 mM Tris/500 mM NaCl/500 mM Imidazole, pH 7.8 and collect elute. Run SDS-PAGE and pool relevant fractions. Concentrate to 2-3 mL and dialyze into 20 mM Tris-HCl/500 mM NaCl, pH 7.8 to remove imidazole.

(a) rsD4DIIIs and rsZDIIIs variants nanoparticle production and biochemical characterization. Protein nanoparticles on the aaLS and hpFer platforms were generated for rsDIII-Ala30 and WT DENV2 EDIII (rsDIII-Ala30 and WT DENV2 EDIII were previously described in PCT/US17/17637 (WO2017/142831); herein incorporated by reference in its entirety). Conjugation efficiency were screened by SDS-PAGE. The rsD4DIIIs and rsZDIIIs variants/nanoparticle component pairs that demonstrate high conjugation efficiency were produced in larger amounts and tested for assembly in vitro to complete nanoparticles. Nanoparticle purity and monodispersity were assessed by SDS-PAGE, UV/vis spectrometry, analytical SEC-MALS, dynamic light scattering, and negative stain electron microscopy. The antigenicity of the displayed antigens were assessed by ELISA using DENV-specific mAbs generated by the Diamond laboratory (Sukupolvi-Petty, S. et al. (2013) J of Virol 87, 8826-8842; Shrestha, B. et al. (2010) PLoS Pathog 6, e1000823; Sukupolvi-Petty, S. et al. (2010) J of Virol 84, 9227-9239; Brien, J. D. et al. (2010) J of Virol 84, 10630-10643). The nanoparticle immunogens were compared to the corresponding soluble (non-particulate) antigen. All assays were run prior to snap-freezing the soluble and nanoparticle immunogens for storage at −80° C. as well as after a freeze/thaw to ensure the integrity of the particles. If many different antigen-bearing particles pass quality control, four candidate immunogens will be selected for immunization studies based on favorable mAb binding profiles, expression and solubility, and nanoparticle homogeneity.

(b) Immunogenicity and challenge experiments. WT DENV2 or rsDIII-Ala30-bearing nanoparticles were used to immunize BALB/c mice as described above and in PCT/US17/17637 (WO2017/142831). In the present studies, Alhydrogel, an Alum-based adjuvant, was used, and, in separate groups, MF59, a squalene-based oil-in-water emulsion. Both adjuvants can be used in humans and non-human primates and M59 in particular has been shown to induce strong B cell responses for subunit vaccines (Lofano, G. et al. (2015) J of immunology (Baltimore, Md.: 1950) 195, 1617-1627). It was anticipated that serum responses will be greater in magnitude relative to monomeric WT DENV2 EDIII and rsDIII-Ala30 owing to the multivalent nature of the antigen presentation. Sera were characterized for binding and neutralization profiles, and the most potent sera tested for protective efficacy in the AG129 mouse model of DENV challenge. Protection against other serotypes will be tested.

Parallel studies with rsD4DIIIs, rsZDIIIs, and multivalent nanoparticles. In parallel, the rsD4DIIIs (Example 1) and rsZDIIIs (Example 2) containing the Spytag were produced and trial conjugations were performed with Spycatcher-bearing nanoparticles as in Example 3. The rsD4DIII- and rsZDIII-bearing nanoparticles (up to four each) were tested for immunogenicity and protection as in Examples 1 and 2, but using alum and MF59 adjuvants.

The nanoparticle rsD4DIIIs variants and rsZDIIIs variants as immunogens induced more robust antibody responses than the corresponding monomeric antigens. Mixtures of nanoparticle immunogens comprising rsDIII-Ala30 as well as the best rsD4DIIIs variants and rsZDIIIs variants were produced to generate multivalent vaccine compositions alone, or conjugated to nanoparticles. These multivalent nanoparticles could induce robust, protective antibody responses against all four DENV serotypes as well as ZIKV. These nanoparticle immunogens were characterized as described above and used for immunization studies.

To enhance immunogenicity of nanoparticle immunogens. The rsD4DIIIs variants and rsZDIIIs variants-bearing nanoparticles were highly immunogenic. To enhance immunogenicity, a 13-amino acid universal Pan HLA-DR Epitope (PADRE) T cell epitope was incorporated into the nanoparticle to augment immune response (Ghaffari-Nazari, H. et al. (2015) PloS one 10; La Rosa, C. et al. (2012) The J of infectious diseases 205, 1294-1304). Other methods to enhance immunicity utilized glycosylated protein nanoparticles, which can more efficiently shuttle to the follicular dendritic cell network and then accumulate in germinal centers, than their unglycosylated counterparts. Other methods to enhance immunicity include engineering site-specific glycosylation sites onto the N-terminus of the rsDIIIs and produce them in mammalian cells. These glycosylated rsD4DIIIs variants and rsZDIIIs variants can then be conjugated to protein nanoparticles and tested in vivo.

In conclusion, the present studies describe the development of novel resurfaced EDIII immunogens for DENV4 and ZIKV (e.g., rsD4DIIIs variants and rsZDIIIs variants). Such rsD4DIIIs variants and rsZDIIIs variants can robustly augment an immune response alone, or when combined in a multivalent vaccine. In addition, when conjugated to nanoparticles, such multivalent nanoparticles show enhanced immunogenicity in mice. These studies lay the groundwork for preclinical development of novel EDIII-based vaccines for DENY and ZIKV in humans and non-humans.

### REFERENCES

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A resurfaced Dengue virus-4 glycoprotein subunit E DIII variant comprising variant rsD4DIII-1 (SEQ ID NO:3) or (SEQ ID NO:69), variant rsD4DIII-2 (SEQ ID NO:4) or (SEQ ID NO:71), variant rsD4DIII-3 (SEQ ID NO:5), variant rsD4DIII-4 (SEQ ID NO:6) or (SEQ ID NO:73), variant rsD4DIII-5 (SEQ ID NO:7), variant rsD4DIII-7 (SEQ ID NO:75), variant rsD4DIII-8 (SEQ ID NO:8) or (SEQ ID NO:77), variant rsD4DIII-9 (SEQ ID NO:9) or (SEQ ID NO:79), variant rsD4DIII-10 (SEQ ID NO:10) or (SEQ ID NO:81), variant rsD4DIII-11 (SEQ ID NO:83), variant rsD4DIII-12 (SEQ ID NO:11), variant rsD4DIII-13 (SEQ ID NO:12), variant rsD4DIII-14 (SEQ ID NO:13) variant rsD4DIII-18 (SEQ ID NO:85).

2. The variant of claim 1 consisting of rsD4DIII-1 (SEQ ID NO:3) or (SEQ ID NO:69).

3. The variant of claim 1 consisting of rsD4DIII-2 (SEQ ID NO:4) or (SEQ ID NO:71).

4. The variant of claim 1 consisting of rsD4DIII-3 (SEQ ID NO:5).

5. The variant of claim 1 consisting of rsD4DIII-4 (SEQ ID NO:6).

6. The variant of claim 1 consisting of rsD4DIII-5 (SEQ ID NO:7).

7. The variant of claim 1 consisting of rsD4DIII-7 (SEQ ID NO:75).

8. The variant of claim 1 consisting of rsD4DIII-8 (SEQ ID NO:8) or (SEQ ID NO:77).

9. The variant of claim 1 consisting of rsD4DIII-9 (SEQ ID NO:9) or (SEQ ID NO:79).

10. The variant of claim 1 consisting of rsD4DIII-10 (SEQ ID NO:10) or (SEQ ID NO:81).

11. The variant of claim 1 consisting of rsD4DIII-11 (SEQ ID NO:83).

12. The variant of claim 1 consisting of rsD4DIII-12 (SEQ ID NO:11).

13. The variant of claim 1 consisting of rsD4DIII-13 (SEQ ID NO:12).

14. The variant of claim 1 consisting of rsD4DIII-14 (SEQ ID NO:13).

15. The variant of claim 1 consisting of rsD4DIII-18 (SEQ ID NO:85).

16. The variant of claim 2, encoded by the nucleic acid set forth in SEQ ID NO:32 or SEQ ID NO: 70.

17. The variant of claim 3, encoded by the nucleic acid set forth in SEQ ID NO:33 or SEQ ID NO: 71.

18. The variant of claim 4, encoded by the nucleic acid set forth in SEQ ID NO:34.

19. The variant of claim 5, encoded by the nucleic acid set forth in SEQ ID NO:35 or SEQ ID NO: 73.

20. The variant of claim 6, encoded by the nucleic acid set forth in SEQ ID NO:36.

21. The variant of claim 7, encoded by the nucleic acid set forth in SEQ ID NO:76.

22. The variant of claim 8, encoded by the nucleic acid set forth in SEQ ID NO:37 or SEQ ID NO: 78.

23. The variant of claim 9, encoded by the nucleic acid set forth in SEQ ID NO:38 or SEQ ID NO: 80.

24. The variant of claim 10, encoded by the nucleic acid set forth in SEQ ID NO:39 or SEQ ID NO: 82.

25. The variant of claim 11, encoded by the nucleic acid set forth in SEQ ID NO:84.

26. The variant of claim 12, encoded by the nucleic acid set forth in SEQ ID NO:40.

27. The variant of claim 13, encoded by the nucleic acid set forth in SEQ ID NO:41.

28. The variant of claim 14, encoded by the nucleic acid set forth in SEQ ID NO:42.

29. The variant of claim 15, encoded by the nucleic acid set forth in SEQ ID NO:86.

30. A resurfaced Zika virus glycoprotein subunit E DIII variant comprising variant rsZDIII-1.20 (SEQ ID NO:18) or (SEQ ID NO:57), variant rsZDIII-1.39 (SEQ ID NO:19) or (SEQ ID NO:59), variant rsZDIII-1.48 (SEQ ID NO:20), variant rsZDIII-1.69 (SEQ ID NO:21), variant rsZDIII-1.74 (SEQ ID NO:22), variant rsZDIII-2.16 (SEQ ID NO:23) or (SEQ ID NO:61), variant rsZDIII-2.39 (SEQ ID NO:24) or (SEQ ID NO:63), variant rsZDIII-2.50 (SEQ ID NO:25), variant rsZDIII-1.8 (SEQ ID NO:26), variant rsZDIII-1.25 (SEQ ID NO:27), or variant rsZDIII-1.27 (SEQ ID NO:28).

31. The variant of claim 30 consisting of rsZDIII-1.20 (SEQ ID NO:18) or (SEQ ID NO:57).

32. The variant of claim 30 consisting of rsZDIII-1.39 (SEQ ID NO:19) or (SEQ ID NO:59).

33. The variant of claim 30 consisting of rsZDIII-1.48 (SEQ ID NO:20).

34. The variant of claim 30 consisting of rsZDIII-1.69 (SEQ ID NO:21).

35. The variant of claim 30 consisting of rsZDIII-1.74 (SEQ ID NO:22).

36. The variant of claim 30 consisting of rsZDIII-2.16 (SEQ ID NO:23) or (SEQ ID NO:61).

37. The variant of claim 30 consisting of rsZDIII-2.39 (SEQ ID NO:24) or (SEQ ID NO:63).

38. The variant of claim 30 consisting of rsZDIII-2.50 (SEQ ID NO:25).

39. The variant of claim 30 consisting of rsZDIII-1.8 (SEQ ID NO:26).

40. The variant of claim 30 consisting of rsZDIII-1.25 (SEQ ID NO:27).

41. The variant of claim 30 consisting of rsZDIII-1.27 (SEQ ID NO:28).

42. The variant of claim 31, encoded by the nucleic acid set forth in SEQ ID NO:44 or SEQ ID NO: 58.

43. The variant of claim 32, encoded by the nucleic acid set forth in SEQ ID NO:45 or SEQ ID NO: 60.

44. The variant of claim 33, encoded by the nucleic acid set forth in SEQ ID NO:46.

45. The variant of claim 34, encoded by the nucleic acid set forth in SEQ ID NO:47.

46. The variant of claim 35, encoded by the nucleic acid set forth in SEQ ID NO:48 or SEQ ID NO: 62.

47. The variant of claim 36, encoded by the nucleic acid set forth in SEQ ID NO:49 or SEQ ID NO: 64.

48. The variant of claim 37, encoded by the nucleic acid set forth in SEQ ID NO:50.

49. The variant of claim 38, encoded by the nucleic acid set forth in SEQ ID NO:51.

50. The variant of claim 39, encoded by the nucleic acid set forth in SEQ ID NO:52

51. The variant of claim 40, encoded by the nucleic acid set forth in SEQ ID NO:53

52. The variant of claim 41, encoded by the nucleic acid set forth in SEQ ID NO:54

53. A dimer or oligomer comprising any of the variants of any of claims 1-29.

54. A dimer or oligomer comprising any of the variants of any of claims 30-52.

55. A virion of an isolated, recombinant Dengue virus comprising a variant of any of claims 1-29 or the dimer or oligomer of claim 53.

56. A Dengue virus vaccine composition comprising a variant of any of claims 1-29, the dimer or oligomer of claim 53, or the virion of claim 55.

57. A virion of an isolated, recombinant Zika virus comprising a variant of any of claims 30-52 or the dimer or oligomer of claim 54.

58. A Zika virus vaccine composition comprising a variant of any of claims 30-52, the dimer or oligomer of claim 54, or the virion of claim 57.

59. The vaccine composition of claim 56 or 58, or combination thereof, further comprising an immunological adjuvant.

60. The vaccine composition of claim 56 or 58, or combination thereof, conjugated to at least one nanoparticle, wherein said composition comprises any of the variants of claim 1-52 engineered with a C-terminus tag comprising SEQ ID NO: 65.

61. A method of eliciting an immune response in a subject comprising administering to the subject (i) the variant of any of claims 1-52, (ii) the dimer or oligomer of claim 53 or 54, (iii) the virion of claim 55 or 57, or (iv) the vaccine of claim 56, 58, 59, or 60 in an amount effective to elicit an immune response in a subject.

62. A method of vaccinating a subject for Dengue virus infection comprising administering to the subject (i) the variant of any of claims 1-29, (ii) the dimer or oligomer of claim 53, (iii) the virion of claim 55, or (iv) the vaccine of claim 56 or 59 in an amount effective to vaccinate a subject for Dengue virus.

63. A method of immunizing a subject against Dengue virus infection comprising administering to the subject (i) the variant of any of claims 1-29, (ii) the dimer or oligomer of claim 53, (iii) the virion of claim 55, or (iv) the vaccine of claim 56 or 59 in an amount effective to immunize a subject against Dengue virus.

64. A method of treating a Dengue virus infection in a subject or treating a disease caused by a Dengue virus infection in a subject comprising administering to the subject (i) the variant of any of claims 1-29, (ii) the dimer or oligomer of claim 53, (iii) the virion of claim 55, or (iv) the vaccine of claim 56 or 59 in an amount effective to treat a Dengue virus infection or treat a disease caused by a Dengue virus infection in a subject.

65. The method of claim 64, wherein the subject has one or more of Dengue Hemorrhagic Fever (DHF) and Dengue Shock Syndrome (DSS).

66. The method of any of claims 61-65, wherein the variant, dimer, oligomer, virion or vaccine is effective against all Dengue virus serotypes.

67. A method of vaccinating a subject for Zika virus infection comprising administering to the subject (i) the variant of any of claims 30-52, (ii) the dimer or oligomer of claim 54, (iii) the virion of claim 57, or (iv) the vaccine of claim 58 or 60 in an amount effective to vaccinate a subject for Zika virus.

68. A method of immunizing a subject against Zika virus infection comprising administering to the subject (i) the variant of any of claims 30-52, (ii) the dimer or oligomer of claim 54, (iii) the virion of claim 57, or (iv) the vaccine of claim 58 or 60 in an amount effective to immunize a subject against Zika virus.

69. A method of treating a Zika virus infection in a subject or treating a disease caused by a Zika virus infection in a subject comprising administering to the subject (i) the variant of any of claims 30-52, (ii) the dimer or oligomer of claim 54, (iii) the virion of claim 57, or (iv) the vaccine of claim 58 or 60 in an amount effective to treat a Zika virus infection or treat a disease caused by a Zika virus infection in a subject.

70. The method of any of claims 67-69, wherein the variant, dimer, oligomer, virion or vaccine is effective against all Zika virus serotypes.
